SEQUENCE LISTING (39 sequences)

SEQ ID NO: 1 - DNA, 201 bp, Artificial Sequence
  EST Clone 1662885
ctcttaggctttgaagcatttttgtntgtgctccctgatcttcatgtcaccaccatgaagttcttagcagtcctggtactcttgggagtttccatctntctggtctctgcccagaatccgacaacagctgctncagctgacacgnatccagctactggtcctgctgatgatgaagcccctgangctgaaaccactgctgct

SEQ ID NO: 2 - DNA, 308 bp, Homo sapiens
taggctttgaagcatttttgtctgtgctccctgatcttcaggtcaccaccatgaagttcttagcagtcctggtactcttgggagtttccatctttctggtctctgcccagaatccgacaacagctgctccagctgacacgtatccagctactggtcctgctgatgatgaagcccctgatgctgaaaccactgctgctgcaaccactgcgaccactgctgctcctaccactgcaaccaccgctgcttctaccactgctcgtaaagacattccagttttacccaaatgggttggggatcttccgaatggt

SEQ ID NO: 3 - DNA, 292 bp, Artificial Sequence
  EST Clone 901429
gcatttttgtctgtgctccctgatcttcatgtcaccaccatgaagttcttagcagtcctggtactcttgggagtttccatctttctggtctctgcccagaatccgacaacagctgctccagctgacacgtatccagctactggtcctgctgatgatgaagcccctgatgctgaaaccactgctgctgcaaccactgcgaccactgctgctcctaccactgcaaccaccgctgcttntaccactgctcgtaaagacattncagttttacccaaatgggttggggatctcccga

SEQ ID NO: 4 - DNA, 197 bp, Homo sapiens
gttttacccaaatgggttggggatctcccgaatggtagagtgtgtccctgagatggaatcagcttgagtcttctgcaattggtcacaactattcatgcttcctgtgatttcatccaactacttaccttgcctacgatatcccctttatctctaatcagtttattttctttcaaataaaaaataactatgagcaacat

SEQ ID NO: 5 - DNA, 472 bp, Homo sapiens
ctcttaggctttgaagcatttttgtctgtgctccctgatcttcatgtcaccaccatgaagttcttagcagtcctggtactcttgggagtttccatctttctggtctctgcccagaatccgacaacagctgctccagctgacacgtatccagctactggtcctgctgatgatgaagcccctgatgctgaaaccactgctgctgcaaccactgcgaccactgctgctcctaccactgcaaccaccgctgcttctaccactgctcgtaaagacattccagttttacccaaatgggttggggatctcccgaatggtagagtgtgtccctgagatggaatcagcttgagtcttctgcaattggtcacaactattcatgcttcctgtgatttcatccaactacttaccttgcctacgatatcccctttatctctaatcagtttattttctttcaaataaaaaataactatgagcaaca

SEQ ID NO: 6 - DNA, 473 bp, Homo sapiens
ctcttaggctttgaagcatttttgtctgtgctccctgatcttcatgtcaccaccatgaagttcttagcagtcctggtactcttgggagtttccatctttctggtctctgcccagaatccgacaacagctgctccagctgacacgtatccagctactggtcctgctgatgatgaagcccctgatgctgaaaccactgctgctgcaaccactgcgaccactgctgctcctaccactgcaaccaccgctgcttctaccactgctcgtaaagacattccagttttacccaaatgggttggggatctcccgaatggtagagtgtgtccctgagatggaatcagcttgagtcttctgcaattggtcacaactattcatgcttcctgtgatttcatccaactacttaccttgcctacgatatcccctttatctctaatcagtttattttctttcaaataaaaaataactatgagcaacat

SEQ ID NO: 7 - DNA, 68 bp, Artificial Sequence
  Restriction site
agctcggaattccgagcttggatcctctagagcggccgccgactagtgagctcgtcgacccgggaatt

SEQ ID NO: 8 - DNA, 68 bp, Artificial Sequence
  Restriction site
aattaattcccgggtcgacgagctcactagtcggcggccgctctagaggatccaagctcggaattccg

SEQ ID NO: 9 - DNA, 24 bp, Artificial Sequence
  Universal Primer
agcggataacaatttcacacagga

SEQ ID NO: 10 - DNA, 18 bp, Artificial Sequence
  Universal Primer
tgtaaaacgacggccagt

SEQ ID NO: 11 - DNA, 20 bp, Artificial Sequence
  Primer
actgctcgtaaagacattcc

SEQ ID NO: 12 - DNA, 19 bp, Artificial Sequence
  Primer
gggacacactctaccattc

SEQ ID NO: 13 - DNA, 20 bp, Artificial Sequence
  Sense Primer
aagcccctgatgctgaaacc

SEQ ID NO: 14 - DNA, 23 bp, Artificial Sequence
  Antisense Primer
tgcagaagactcaagctgattcc

SEQ ID NO: 15 - DNA, 20 bp, Artificial Sequence
  Target-Specific Forward Primer
aagcccctgatgctgaaacc

SEQ ID NO: 16 - DNA, 23 bp, Artificial Sequence
  Target-Specific Reverse Primer
tgcagaagactcaagctgattcc

SEQ ID NO: 17 - DNA, 15 bp, Artificial Sequence
  Probe
gaccactgctgctcc

SEQ ID NO: 18 - DNA, 20 bp, Artificial Sequence
  Sense Primer
actgctcgtaaagacattcc

SEQ ID NO: 19 - DNA, 19 bp, Artificial Sequence
  Antisense Primer
gggacacactctaccattc

SEQ ID NO: 20 - protein, 90 aa, Homo sapiens
MKFLAVLVLLGVSIFLVSAQNPTTAAPADTYPATGPADDEAPDAETTAAATTATTAAPTTATTAASTTARKDIPVLPKWVGDLPNGRVCP

SEQ ID NO: 21 - protein, 39 aa, Artificial Sequence
  Synthetic Peptide
AQNPTTAAPADTYPATGPADDEAPDAETTAAATTATTAA

SEQ ID NO: 22 - protein, 39 aa, Artificial Sequence
  Synthetic Peptide
TTATTAAPTTATTAASTTARKDIPVLPKWVGDLPNGRVC

SEQ ID NO: 23 - protein, 21 aa, Artificial Sequence
  Synthetic Peptide
ARKDIPVLPKWVGDLPNGRVC

SEQ ID NO: 24 - protein, 21 aa, Artificial Sequence
  Synthetic Peptide
AAPADTYPATGPADDEAPDAE

SEQ ID NO: 25 - protein, 9 aa, Artificial Sequence
  Synthetic Peptide
AQNPTTAAC

SEQ ID NO: 26 - protein, 23 aa, Artificial Sequence
  Synthetic Peptide
CARKDIPVLPKWVGDLPNGRVCP

SEQ ID NO: 27 - protein, 14 aa, Artificial Sequence
  Synthetic Peptide
GGWVGDLPNGRVCP

SEQ ID NO: 28 - protein, 12 aa, Artificial Sequence
  Synthetic Peptide
GPADDEAPDAEC

SEQ ID NO: 29 - protein, 40 aa, Artificial Sequence
  Synthetic Peptide
AQNPTTAAPADTYPATGPADDEAPDAETTAAATTATTAAC

SEQ ID NO: 30 - protein, 11 aa, Artificial Sequence
  Synthetic Peptide
QNPTTAAPADC

SEQ ID NO: 31 - protein, 10 aa, Artificial Sequence
  Synthetic Peptide
NPTTAAPADC

SEQ ID NO: 32 - protein, 11 aa, Artificial Sequence
  Synthetic Peptide
PTTAAPADTYC

SEQ ID NO: 33 - protein, 22 aa, Artificial Sequence
  Synthetic Peptide
ARKDIPVLPKWVGDLPNGRVCP

SEQ ID NO: 34 - protein, 24 aa, Artificial Sequence
  Affinity Purification System Recognition Site
ASPTYRLYSASPASPASPASPLYS

SEQ ID NO: 35 - protein, 57 aa, Artificial Sequence
  Affinity Purification System Recognition Site
GLGLNLYSLEILESERGLGLASPLEASNMETHISTHRGLHISHISHISHISHISHIS

SEQ ID NO: 36 - DNA, 36 bp, Artificial Sequence
  BamH I site
tccatctttctggtcggatcccagaatccgacaaca

SEQ ID NO: 37 - DNA, 35 bp, Artificial Sequence
  Pme I site
gagcggccgcatcgtttaaactgacgatctgcctc

SEQ ID NO: 38 - protein, 4 aa, Artificial Sequence
  XbaI site plus 12 nucleotide sequences that encode the four amino acid sequences
SNEL

SEQ ID NO: 39 - protein, 8 aa, Artificial Sequence
  Antisense primer incorporates a sequence encoding the eight amino acids just before the stop codons
DYKDDDDK

(19) United States
(12) Patent Application Publication
(10) Pub. No.: US 2005/0153373 A1
(43) Pub. Date: Jul. 14, 2005

(54) Reagents and methods useful for detecting diseases of the breast

(76) Inventors: Patricia A. Billing-Medel, Grayslake, IL (US); Maurice Cohen, Highland Park, IL (US); Tracey L. Colpitts, Round Lake, IL (US); Paula N. Friedman, Deerfield, IL (US); Julian Gordon, Lake Bluff, IL (US); Edward N. Granados, Vernon Hills, IL (US); Steven C. Hodges, Buffalo Grove, IL (US); Michael R. Klass, Libertyville, IL (US); Jon D. Kratochvil, Wildwood, MO (US); Lisa Roberts-Rapp, Gurnee, IL (US); John C. Russell, Pleasant Prairie, WI (US); Stephen D. Stroupe, Libertyville, IL (US)

Correspondence Address: Steven F. Weinstock;Abbott Laboratories, D-377/AP6D, 100 Abbott Park Road, Abbott Park, IL, 60064-3500, US

(21) Appl. No.: 10/991,681

(22) Filed: Nov. 18, 2004

Related U.S. Application Data
(63) Continuation of application No. 09/516,444, filed on Feb. 29, 2000, now abandoned.
Application No. 09/516,444 is a continuation-in-part of application No. 08/962,094, filed on Oct. 31, 1997, now abandoned.
Application No. 08/962,094 is a continuation-in-part of application No. 08/742,067, filed on Oct. 31, 1996, now abandoned.

(51) Int. Cl. G01N 33/574; C07K 16/30; C07K 14/705; C12N 5/06

(52) U.S. Cl. 435/7.23; 530/350; 530/388.8; 435/334

(57) ABSTRACT

A set of contiguous and partially overlapping RNA sequences and polypeptides encoded thereby, designated as BS106 and transcribed from breast tissue is described. These sequences are useful for the detecting, diagnosing, staging, monitoring, prognosticating, preventing or treating, or determining the predisposition of an individual to diseases and conditions of the breast such as breast cancer. Also provided are antibodies which specifically bind to BS106-encoded polypeptide or protein, and agonists or inhibitors which prevent action of the tissue-specific BS106 polypeptide, which molecules are useful for the therapeutic treatment of breast diseases, tumors or metastases.

### CROSS-REFERENCE TO RELATED APPLICATION

This application is a continuation-in-part of U.S. application Ser. No. 08/742,067, filed Oct. 31, 1996, and U.S. Application Ser. No. 08/962,094, filed Oct. 31, 1997, from which priority is claimed pursuant to 35 U.S.C. §120 and which are incorporated herein by reference in its entirety.

### BACKGROUND OF THE INVENTION

The invention relates generally to detecting diseases of the breast, and more particularly, relates to reagents such as polynucleotide sequences and the polypeptide sequences encoded thereby, as well as methods which utilize these sequences, which are useful for detecting, diagnosing, staging, monitoring, prognosticating, preventing or treating, or determining predisposition to diseases or conditions of the breast such as breast cancer.

Breast cancer is the most common form of cancer occurring in females in the US. The incidence of breast cancers in the United States is projected to be 175,000 cases diagnosed and 43,300 breast cancer related deaths to occur during 1999 (American Cancer Society statistics). Worldwide, the incidence of breast cancer has increased from 700,000 in 1985 to about 900,000 in 1990. G. N. Hortobagyi et al., CA Cancer J Clin 45: 199-226 (1995).

Procedures used for detecting, diagnosing, staging, monitoring, prognosticating, preventing or treating, or determining predisposition to diseases or conditions of the breast such as breast cancer are of critical importance to the outcome of the patient. For example, patients diagnosed with early breast cancer have greater than a 90% five-year relative survival rate as compared to a survival rate of about 20% for patients diagnosed with distantly metastasized breast cancers. (American Cancer Society statistics). Currently, the best initial indicators of early breast cancer are physical examination of the breast and mammography. J. R. Harris et al. In: Cancer: Principles and Practice of Oncology, Fourth Edition, pp. 1264-1332, Philadelphia, Pa.: J/B. Lippincott Co. (1993). Mammography may detect a breast tumor before it can be detected by physical examination, but it has limitations. For example, mammography's predictive value depends on the observer's skill and the quality of the mammogram. In addition, 80 to 93% of suspicious mammograms are false positives, and 10 to 15% of women with breast cancer have false negative mammograms. C. J. Wright et al., Lancet 346: 29-32 (1995). New diagnostic methods which are more sensitive and specific for detecting early breast cancer are clearly needed.

Breast cancer patients are closely monitored following initial therapy and during adjuvant therapy to determine response to therapy, and to detect persistent or recurrent disease, or early distant metastasis; Current diagnostic procedures for monitoring breast cancer include mammography, bone scan, chest radiographs, liver function tests and tests for serum markers. The serum tumor markers most commonly used for monitoring patients are carcinoembryonic antigen (CEA) and CA 15-3. Limitations of CEA include absence of elevated serum levels in about 40% of women with metastatic disease. In addition, CEA elevation during adjuvant therapy may not be related to recurrence but to other factors that are not clinically important. CA 15-3 can also be negative in a significant number of patients with progressive disease and, therefore, fails to predict metastasis. Both CEA and CA 15-3 can be elevated in nonmalignant, benign conditions giving rise to false positive results. Therefore, it would be clinically beneficial to find a breast associated marker which is more sensitive and specific in detecting cancer recurrence. J. R. Harris, et al., supra. M. K. Schwartz, In: Cancer: Principles and Practice of Oncology, Vol. 1, Fourth Edition, pp. 531-542, Philadelphia, Pa.: J/B. Lippincott Co. 1993.

Another important step in managing breast cancer is to determine the stage of the patient's disease, because it has potential prognostic value and provides criteria for designing optimal therapy. Currently, pathological staging of breast cancer is preferable over clinical staging because the former gives a more accurate prognosis. J. R. Harris, et al., supra. On the other hand, clinical staging would be preferred were it at least as accurate as pathological staging, because it does not depend on an invasive procedure to obtain tissue for pathological evaluation. Staging of breast cancer could be improved by detecting new markers in serum or urine which could differentiate between different stages of invasion. Such markers could be mRNA or protein markers expressed by cells originating from the primary tumor in the breast but residing in blood, bone marrow or lymph nodes and could serve as sensitive indicators for metastasis to these distal organs. For example, specific protein antigens and mRNA, associated with breast epithelial cells, have been detected by immunohistochemical techniques and RT-PCR, respectively, in bone marrow, lymph nodes and blood of breast cancer patients suggesting metastasis. K. Pantel, et al., Onkologie 18: 394-401 (1995).

Such procedures also could include assays based upon the appearance of various disease markers in test samples such as blood, plasma, serum, or urine obtained by minimally invasive procedures which are detectable by immunological methods. These procedures would provide information to aid the physician in managing the patient with disease of the breast at low cost to the patient. Markers such as prostate specific antigen (PSA) and human chorionic gonadotropin (hCG) exist and are used clinically for screening patients for prostate cancer and testicular cancer, respectively. For example, PSA normally is secreted by the prostate at high levels into the seminal fluid, but is present in very low levels in the blood of men with normal prostates. Elevated levels of PSA protein in serum are used in the early detection of prostate cancer or disease in asymptomatic men. See, for example, G. E. Hanks, et al., In: Cancer: Principles and Practice of Oncology, Vol. 1, Fourth Edition, pp. 1073-1113, Philadelphia, Pa.: J. B. Lippincott Co. 1993. M. K. Schwartz, et al., In: Cancer: Principles and Practice of Oncology, Vol. 1, Fourth Edition, pp. 531-542, Philadelphia, Pa.: J. B. Lippincott Co. 1993. Likewise, the management of breast diseases could be improved by the use of new markers normally expressed in the breast but found in elevated amounts in an inappropriate body compartment as a result of the disease of the breast.

Further, new markers which could predict the biologic behavior of early breast cancers would also be of significant value. Early breast cancers that threaten or will threaten the life of the patient are more clinically important than those that do not or will not be a threat. G. E. Hanks, supra. Such markers are needed to predict which patients with histologically negative lymph nodes will experience recurrence of cancer and also to predict which cases of ductal carcinoma in situ will develop into invasive breast carcinoma. More accurate prognostic markers would allow the clinician to accurately identify early cancers localized to the breast which will progress and metastasize if not treated aggressively. Additionally, the absence of a marker for an aggressive cancer in the patient could spare the patient expensive and non-beneficial treatment. J. R. Harris, et al., supra. E. R. Frykberg, et al., Cancer 74: 350-361 (1994).

It would be advantageous, therefore, to provide specific methods and reagents useful for detecting, diagnosing, staging, monitoring, prognosticating, preventing or treating, or determining predisposition to diseases or conditions of the breast. Such methods would include assaying a test sample for products of a gene which are overexpressed in diseases and conditions associated with the breast including cancer. Such methods may also include assaying a test sample for products of a gene which have been altered by the disease or condition associated with the breast including cancer. For example, these assays would include methods for detecting the gene products (proteins) in light of possible post-translational modifications that can occur in the body. Such post-translational modifications can include proteolytic processing, alteration of the chain termini, glycosylation, lipid attachment, sulfation, gamma-carboxylation, hydroxylation, phosphorylation, ADP-ribosylation, disulfide bond formation, transglutamination, and multiple non-covalent interactions with molecules such as co-factors, inhibitors (both small molecule and protein), activators (both small molecule and protein), and other proteins in formation of multi-subunit complexes. See, for example, T. E. Creighton et al., In: Proteins: Structures and Molecular Properties, Second Edition, pp. 78-102, New York, N.Y.: W. H. Freeman and Co. 1993. Some modifications are sequence specific and are therefore predictive whereas others are not and are observed by empirical data only. Such methods may further include assaying a test sample for products of a gene whose distribution among the various tissues and compartments of the body have been altered by a breast-associated disease or condition including cancer. Such methods would comprise making cDNA from mRNA in the test sample, amplifying, when necessary, portions of the cDNA corresponding to the gene or a fragment thereof, and detecting the cDNA product as an indication of the presence of the disease or condition including cancer or detecting translation products of the mRNAs comprising gene sequences as an indication of the presence of the disease. Useful reagents include polynucleotides, or fragments thereof which may be used in diagnostic methods such as reverse transcriptase-polymerase chain reaction (RT-PCR), PCR, or hybridization assays of mRNA extracted from biopsied tissue, blood or other test samples; or proteins which are the translation products of such mRNAs; or antibodies directed against these proteins. Such assays would include methods for assaying a sample for products of the gene and detecting the products as an indication of disease of the breast. Drug treatment or gene therapy for diseases and conditions of the breast including cancer can be based on these identified gene sequences or their expressed proteins, and efficacy of any particular therapy can be monitored. Furthermore, it would be advantageous to have available alternative, non-surgical diagnostic methods capable of detecting early stage breast disease such as cancer.

### SUMMARY OF THE INVENTION

The present invention provides a method of detecting a target BS106 polynucleotide in a test sample which comprises contacting the test sample with at least one BS106-specific polynucleotide and detecting the presence of the target BS106 polynucleotide in the test: sample. The BS106-specific polynucleotide has at least 50% identity with a polynucleotide selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof. Also, the BS106-specific polynucleotide may be attached to a solid phase prior to performing the method.

The present invention also provides a method for detecting BS106 mRNA in a test sample, which comprises performing reverse transcription (RT) with at least one primer in order to produce cDNA, amplifying the cDNA so obtained using BS106 oligonucleotides as sense and antisense primers to obtain BS106 amplicon, and detecting the presence of the BS106 amplicon as an indication of the presence of BS106 mRNA in the test sample, wherein the BS106 oligonucleotides have at least 50% identity to a sequence selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof. Amplification can be performed by the polymerase chain reaction. Also, the test sample can be reacted with a solid phase prior to performing the method, prior to amplification or prior to detection. This reaction can be a direct or an indirect reaction. Further, the detection step can comprise utilizing a detectable label capable of generating a measurable signal. The detectable label can be attached to a solid phase.

The present invention further provides a method of detecting a target BS106 polynucleotide in a test sample suspected of containing target BS106 polynucleotides, which comprises (a) contacting the test sample with at least one BS106 oligonucleotide as a sense primer and at least one BS106 oligonucleotide as an anti-sense primer, and amplifying same to obtain a first stage reaction product; (b) contacting the first stage reaction product with at least one other BS106 oligonucleotide to obtain a second stage reaction product, with the proviso that the other BS106 oligonucleotide is located 3′ to the BS106 oligonucleotides utilized in step (a) and is complementary to the first stage reaction product; and (c) detecting the second stage reaction product as an indication of the presence of a target BS106 polynucleotide in the test sample. The BS106 oligonucleotides selected as reagents in the method have at least 50% identity to a sequence selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof. Amplification may be performed by the polymerase chain reaction. The test sample can be reacted either directly or indirectly with a solid phase prior to performing the method, or prior to amplification, or prior to detection. The detection step also comprises utilizing a detectable label capable of generating a measurable signal; further, the detectable label can be attached to a solid phase. Test kits useful for detecting target BS106 polynucleotides in a test sample are also provided which comprise a container containing at least one BS106-specific polynucleotide selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof. These test kits further comprise containers with tools useful for collecting test samples (such as, for example, blood, urine, saliva and stool). Such tools include lancets and absorbent paper or cloth for collecting and stabilizing blood; swabs for collecting and stabilizing saliva; and cups for collecting and stabilizing urine or stool samples. Collection materials, such as papers, cloths, swabs, cups and the like, may optionally be treated to avoid denaturation or irreversible adsorption of the sample. The collection materials also may be treated with or contain preservatives, stabilizers or antimicrobial agents to help maintain the integrity of the specimens.

The present invention provides a purified polynucleotide or fragment thereof derived from a BS106 gene. The purified polynucleotide is capable of selectively hybridizing to the nucleic acid of the BS106 gene, or a complement thereof. The polynucleotide has at least 50% identity to a polynucleotide selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof. Further, the purified polynucleotide can be produced by recombinant and/or synthetic techniques. The purified recombinant polynucleotide can be contained within a recombinant vector. The invention further comprises a host cell transfected with said vector.

The present invention further provides a recombinant expression system comprising a nucleic acid sequence that includes an open reading frame derived from BS106. The nucleic acid sequence has at least 50% identity with a sequence selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof. The nucleic acid sequence is operably linked to a control sequence compatible with a desired host. Also provided is a cell transfected with this recombinant expression system.

The present invention also provides polypeptides encoded by BS106. The polypeptides can be produced by recombinant technology, provided in purified form, or produced by synthetic techniques. The polypeptides comprise amino acid sequences which have at least 50% identity to an amino acid sequence selected from the group consisting of SEQUENCE ID NOS. 20-33.

Also provided is an antibody which specifically binds to at least one BS106 epitope. The antibody can be a polyclonal or monoclonal antibody. The epitope is derived from an amino acid sequence selected from the group consisting of SEQUENCE ID NOS. 20-33, and fragments thereof. Assay kits for determining the presence of BS106 antigen or anti-BS106 antibody in a test sample are also included. In one embodiment, the assay kits comprise a container containing at least one BS106 polypeptide having at least 50% identity to an amino acid sequence selected from the group consisting of SEQUENCE ID NOS. 20-33, and fragments thereof. Further, the test kit can comprise a container with tools useful for collecting test samples (such as blood, urine, saliva and stool). Such tools include lancets and absorbent paper or cloth for collecting and stabilizing blood; swabs for collecting and stabilizing saliva; and cups for collecting and stabilizing urine or stool samples. Collection materials such as, papers, cloths, swabs, cups and the like, may optionally be treated to avoid denaturation or irreversible adsorption of the sample. These collection materials also may be treated with or contain preservatives, stabilizers or antimicrobial agents to help maintain the integrity of the specimens. Also, the polypeptide can be attached to a solid phase.

In another embodiment of the invention, antibodies or fragments thereof against the BS106 antigen can be used to detect or image localization of the antigen in a patient for the purpose of detecting or diagnosing a disease or condition. Such antibodies can be polyclonal or monoclonal, or made by molecular biology techniques, and can be labeled with a variety of detectable labels, including but not limited to radioisotopes and paramagnetic metals. Furthermore, antibodies or fragments thereof, whether monoclonal, polyclonal, or made by molecular biology techniques, can be used as therapeutic agents for the treatment of diseases characterized by expression of the BS106 antigen. In the case of therapeutic applications, the antibody may be used without derivitization, or it may be derivitized with a cytotoxic agent such as a radioisotope, enzyme, toxin, drug, prodrug, or the like.

Another assay kit for determining the presence of BS106 antigen or anti-BS106 antibody in a test sample comprises a container containing an antibody which specifically binds to a BS106 antigen, wherein the BS106 antigen comprises at least one BS106-encoded epitope. The BS106 antigen has at least about 60% sequence similarity to a sequence of a BS106-encoded antigen selected from the group consisting of SEQUENCE ID NOS. 20-33, and fragments thereof. These test kits can further comprise containers with tools useful for collecting test samples (such as blood, urine, saliva and stool). Such tools include lancets and absorbent paper or cloth for collecting and stabilizing blood; swabs for collecting and stabilizing saliva; cups for collecting and stabilizing urine or stool samples. Collection materials, papers, cloths, swabs, cups and the like, may optionally be treated to avoid denaturation or irreversible adsorption of the sample. These collection materials also may be treated with, or contain, preservatives, stabilizers or antimicrobial agents to help maintain the integrity of the specimens. The antibody can be attached to a solid phase.

A method for producing a polypeptide which contains at least one epitope of BS106 is provided, which method comprises incubating host cells transfected with an expression vector. This vector comprises a polynucleotide sequence encoding a polypeptide, wherein the polypeptide comprises an amino acid sequence having at least 50% identity to a BS106 amino acid sequence selected from the group consisting of SEQUENCE ID NOS. 20-33, and fragments thereof.

A method for detecting BS106 antigen in a test sample suspected of containing BS106 antigen also is provided. The method comprises contacting the test sample with an antibody or fragment thereof which specifically binds to at least one epitope of a BS106 antigen, for a time and under conditions sufficient for the formation of antibody/antigen complexes; and detecting the presence of such complexes containing the antibody as an indication of the presence of BS106 antigen in the test sample. The antibody can be attached to a solid phase and be either a monoclonal or polyclonal antibody. Furthermore, the antibody specifically binds to at least one BS106 antigen selected from the group consisting of SEQUENCE ID NOS. 20-33, and fragments thereof.

Another method is provided which detects antibodies which specifically bind to BS106 antigen in a test sample suspected of containing these antibodies. The method comprises contacting the test sample with a polypeptide which contains at least one BS106 epitope, wherein the BS106 epitope comprises an amino acid sequence having at least 50% identity with an amino acid sequence encoded by a BS106 polynucleotide, or a fragment thereof. Contacting is carried out for a time and under conditions sufficient to allow antigen/antibody complexes to form. The method further entails detecting complexes which contain the polypeptide. The polypeptide can be attached to a solid phase. Further, the polypeptide can be a recombinant protein or a synthetic peptide having at least 50% identity to an amino acid sequence selected from the group consisting of SEQUENCE ID NOS. 20-33, and fragments thereof.

The present invention provides a cell transfected with a BS106 nucleic acid sequence that encodes at least one epitope of a BS106 antigen, or fragment thereof. The nucleic acid sequence is selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof.

A method for producing antibodies to BS106 antigen also is provided, which method comprises administering to an individual an isolated immunogenic polypeptide or fragment thereof, wherein the isolated immunogenic polypeptide comprises at least one BS106 epitope in an amount sufficient to produce an immune response. The isolated, immunogenic polypeptide comprises an amino acid sequence selected from the group consisting of SEQUENCE ID NOS. 20-33, and fragments thereof.

Another method for producing antibodies which specifically bind to BS106 antigen is disclosed, which method comprises administering to a mammal a plasmid comprising a nucleic acid sequence which encodes at least one BS106 epitope derived from an amino acid sequence selected from the group consisting of SEQUENCE ID NOS. 20-33, and fragments thereof. The plasmid is administered in an amount such that the plasmid is taken up by cells in the individual and expressed at levels sufficient to produce an immune response.

Also provided is a composition of matter that comprises a BS106 polynucleotide of at least about 10-12 nucleotides having at least 50% identity to a polynucleotide selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof. The BS106 polynucleotide encodes an amino acid sequence having at least one BS106 epitope. Another composition of matter provided by the present invention comprises a polypeptide with at least one BS106 epitope of about 8-10 amino acids. The polypeptide comprises an amino acid sequence having at least 50% identity to an amino acid sequence selected from the group consisting of SEQUENCE ID NOS. 20-33, and fragments thereof. Also provided is a gene, or fragment thereof, coding for a BS106 polypeptide which has at least 50% identity to SEQUENCE ID NO 20; and a gene, or a fragment thereof, comprising DNA having at least 50% identity to SEQUENCE ID NO 5 or SEQUENCE ID NO 6.

### BRIEF DESCRIPTION OF THE DRAWINGS

FIG. 1 shows the nucleotide alignment of clones 1662885 (SEQUENCE ID NO 1), 893988 (SEQUENCE ID NO 2), 901429 (SEQUENCE ID NO 3), 1209814 (SEQUENCE ID NO 4), the full-length sequence of clone 1662885 [designated as 1662885inh (SEQUENCE ID NO 5)], and the consensus sequence (SEQUENCE ID NO 6) derived therefrom.

FIG. 2 shows the contig map depicting the formation of the consensus nucleotide sequence (SEQUENCE ID NO 6) from the nucleotide alignment of overlapping clones 1662885 (SEQUENCE ID NO 1), 892988 (SEQUENCE ID NO 2), 901429 (SEQUENCE ID NO 3), 1209814 (SEQUENCE ID NO 4), 1662885inh (SEQUENCE ID NO 5).

FIG. 3A contains a scan of an ethidium bromide stained agarose gel of RNA and the corresponding Northern blot of RNA following hybridization with a BS106 radiolabeled probe. Samples include from breast tissues and prostate tissue extracts.

FIG. 3B contains a scan of an ethidium bromide stained agarose gel of RNA and the corresponding Northern blot of RNA following hybridization with a BS106 radiolabeled probe. Samples include from normal breast tissues and breast cancer tissue extracts.

FIG. 4 shows the results of a BS106 probe against a Clontech (Clontech Laboratories, Inc., Palo Alto, Calif.) Multiple Tissue Expression Array™ containing polyA RNA from 76 different human tissues.

FIG. 5A is a scan of a SYBR® Green stained agarose gel of BS106 RNA-specific PCR amplification products. It shows a DNA band at 201 bases that is indicative of a BS106 mRNA-specific RT-PCR product in normal breast tissue samples and in breast cancer tissue samples.

FIG. 5B is a scan of a SYBR® Green stained agarose gel of BS106 RNA-specific PCR amplification products. The 201 bases band which is indicative of a BS106 mRNA-specific RT-PCR product is absent in colon tissue samples and lung tissue samples.

FIG. 6 shows the BS106 amplicon was easily detected at 18 picograms of RNA from the MDA 361 cell line using a LCx® system (Abbott Laboratories, Abbott Park, Ill.) assay.

FIG. 7 shows the overall tissue distribution for the BS106 marker using a LCx® system assay.

FIG. 8 shows a Western blot of HEK293 cell products with a BS106 anti-myc epitope monoclonal antibody.

FIG. 9 shows a dot blot of fractions collected from Nickel chelate column using a monoclonal antibody recognizing a myc epitope.

FIG. 10 shows a Western blot of pooled, dialyzed, semi-purified supernatant analyzed for the presence of BS106 M/H.

FIG. 11 shows pooled, purified BS106 M/H analysed by Western blot using both an anti-myc monoclonal antibody in panel A and an anti-BS106 polyclonal antisera in panel B.

FIG. 12 shows the results of a Western blot performed on a panel of tissue extracts using a monoclonal antibody (H9C29) directed against BS106 peptide SEQUENCE ID NO 26. Each lane of FIG. 12 contains a different tissue extract.

FIG. 13 shows Western blots of reduced and unreduced human milk fractions against the affinity purified rabbit anti-BS106 antibody. Panel B shows the results of a competitive reaction with the disappearance of the bands seen in Panel A in the presence of BS106 peptide (SEQUENCE ID NO 26).

FIG. 14 shows Western blots of biological samples with BS106 antibodies.

FIG. 15A-C shows samples of the BS106 fluorescein conjugates titrated with the affinity purified rabbit anti-BS106 and the mouse BS106 monoclonal antibodies H39C51 and H9C29.

### DETAILED DESCRIPTION OF THE INVENTION

The present invention provides a gene or a fragment thereof which codes for a BS106 polypeptide having at least about 50% identity to SEQUENCE ID NO 20. The present invention further encompasses a BS106 gene or a fragment thereof comprising DNA which has at least about 50% identity to SEQUENCE ID NO 5 or SEQUENCE ID NO 6.

The present invention provides methods for assaying a test sample for products of a breast tissue gene designated as BS106, which method comprises making cDNA from mRNA in the test sample and detecting the cDNA as an indication of the presence of breast tissue gene BS106. The method may include an amplification step, wherein one or more portions of the mRNA from BS106 corresponding to the gene or fragments thereof, is amplified. Methods also are provided for assaying for the translation products of BS106. Test samples which may be assayed by the methods provided herein include tissues, cells, body fluids and secretions. The present invention also provides reagents such as oligonucleotide primers and polypeptides which are useful in performing these methods.

Portions of the nucleic acid sequences disclosed herein are useful as primers for the reverse transcription of RNA or for the amplification of cDNA; or as probes to determine the presence of certain mRNA sequences in test samples. Also disclosed are nucleic acid sequences which permit the production of encoded polypeptide sequences which are useful as standards or reagents in diagnostic immunoassays, as targets for pharmaceutical screening assays and/or as components or as target sites for various therapies. Monoclonal and polyclonal antibodies directed against at least one epitope contained within these polypeptide sequences are useful as delivery agents for therapeutic agents as well as for diagnostic tests and for screening for diseases or conditions associated with BS106, especially breast cancer. Isolation of sequences of other portions of the gene of interest can be accomplished utilizing probes or PCR primers derived from these nucleic acid sequences. This allows additional probes of the mRNA or cDNA of interest to be established, as well as corresponding encoded polypeptide sequences. These additional molecules are useful in detecting, diagnosing, staging, monitoring, prognosticating, preventing or treating, or determining the predisposition to, diseases and conditions of the breast such as breast cancer, characterized by BS106, as disclosed herein.

The compositions and methods described herein will enable the identification of certain markers as indicative of a breast tissue disease or condition; the information obtained therefrom will aid in the detecting, diagnosing, staging, monitoring, prognosticating, preventing or treating, or determining diseases or conditions associated with BS106, especially breast cancer. Test methods include, for example, probe assays which utilize the sequence(s) provided herein and which also may utilize nucleic acid amplification methods such as the polymerase chain reaction (PCR), the ligase chain reaction (LCR), and hybridization.

In addition, the nucleotide sequences provided herein contain open reading frames from which an immunogenic epitope may be found. This epitope is believed to be unique to the disease state or condition associated with BS106. It also is thought that the polynucleotides or polypeptides and protein encoded by the BS106 gene are useful as a marker. This marker is either elevated in disease such as breast cancer, altered in disease such as breast cancer, or present as a normal protein but appearing in an inappropriate body compartment. The uniqueness of the epitope may be determined by its immunological reactivity and specificity with antibodies directed against proteins and polypeptides encoded by the BS106 gene, and (ii) its nonreactivity with any other tissue markers. Methods for determining immunological reactivity are well-known and include but are not limited to, for example, radioimmunoassay (RIA), enzyme-linked immunosorbent assay (ELISA), hemagglutination (HA), fluorescence polarization immunoassay (FPIA), chemiluminescent immunoassay (CLIA) and others. Several examples of suitable methods are described herein.

Unless otherwise stated, the following terms shall have the following meanings:
Techniques for determining amino acid sequence “similarity” are well known in the art. In general, “similarity” means the exact amino acid to amino acid comparison of two or more polypeptides at the appropriate place, where amino acids are identical or possess similar chemical and/or physical properties such as charge or hydrophobicity. A so-termed “percent similarity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby, and comparing this to a second amino acid sequence. In general, “identity” refers to an exact nucleotide to nucleotide or amino acid to amino acid correspondence of two polynucleotides or polypeptide sequences, respectively.

Two or more polynucleotide sequences can be compared by determining their “percent identity.” Two or more amino acid sequences likewise can be compared by determining their “percent identity.” The percent identity of two sequences, whether nucleic acid or peptide sequences, is the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2: 482-489 (1981). This algorithm can be extended to use with peptide sequences using the scoring matrix developed by Dayhoff, Atlas of Protein Sequences and Structure, M. O. Dayhoff ed., 5 suppl. 3: 353-358, National Biomedical Research Foundation, Washington, D.C., USA, and normalized by Gribskov, Nucl. Acids Res. 14(6): 6745-6763 (1986). An implementation of this algorithm for nucleic acid and peptide sequences is provided by the Genetics Computer Group (Madison, Wis.) in their BestFit utility application. The default parameters for this method are described in the Wisconsin Sequence Analysis Package Program Manual, Version 8 (1995) (available from Genetics Computer Group, Madison, Wis.). Other equally suitable programs for calculating the percent identity or similarity between sequences are generally known in the art.

For example, percent identity of a particular nucleotide sequence to a reference sequence can be determined using the homology algorithm of Smith and Waterman with a default scoring table and a gap penalty of six nucleotide positions. Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages, the Smith-Waterman algorithm can be employed where default parameters are used for the scoring table (for example, gap open penalty of 12, gap extension penalty of one, and a gap of six). From the data generated, the “Match” value reflects “sequence identity.” Other suitable programs for calculating the percent identity or similarity between sequences are generally known in the art, such as the alignment program BLAST, which can also be used with default parameters. For example, BLASTN and BLASTP can be used with the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

One of skill in the art can readily determine the proper search parameters to use for a given sequence in the above programs. For example, the search parameters may vary based on the size of the sequence in question. Thus, for example, a representative embodiment of the present invention would include an isolated BS106 polynucleotide having X contiguous nucleotides, wherein (i) the X contiguous nucleotides have at least about 50% identity to Y contiguous nucleotides derived from any of SEQUENCE ID NO 1, SEQUENCE ID NO 2, SEQUENCE ID NO 3, SEQUENCE ID NO 4, SEQUENCE ID NO 5, SEQUENCE ID NO 6, (ii) X equals Y, and (iii) X is greater than or equal to 6 nucleotides and up to 5000 nucleotides, preferably greater than or equal to 8 nucleotides and up to 5000 nucleotides, more preferably 10-12 nucleotides and up to 5000 nucleotides, and even more preferably 15-20 nucleotides, up to the number of nucleotides present in the full-length BS106 sequence described herein, including all integer values falling between the above-described ranges.

A polynucleotide “derived from” or “specific for” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The sequence may be complementary or identical to a sequence which is unique to a particular polynucleotide sequence as determined by techniques known in the art. Comparisons to sequences in databanks, for example, can be used as a method to determine the uniqueness of a designated sequence. Regions from which sequences may be derived, include but are not limited to, regions encoding specific epitopes, as well as non-translated and/or non-transcribed regions.

The derived polynucleotide will not necessarily be derived physically from the nucleotide sequence of interest under study, but may be generated in any manner, including but not limited to chemical synthesis, replication, reverse transcription or transcription, which is based on the information provided by the sequence of bases in the region(s) from which the polynucleotide is derived. As such, it may represent either a sense or an antisense orientation of the original polynucleotide. In addition, combinations of regions corresponding to that of the designated sequence may be modified in ways known in the art to be consistent with the intended use.

A “fragment” of a specified polynucleotide refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the specified nucleotide sequence.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., PNA as defined hereinbelow) which can be used to identify a specific polynucleotide present in samples bearing the complementary sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences which are immunologically identifiable with a polypeptide encoded by the sequence. Thus, a “polypeptide,” “protein,” or “amino acid” sequence has at least about 50% identity, preferably about 60% identity, more preferably about 75-85% identity, and most preferably about 90-95% or more identity to a BS106 amino acid sequence. Further, the BS106 “polypeptide,” “protein,” or “amino acid” sequence may have at least about 60% similarity, preferably at least about 75% similarity, more preferably about 85% similarity, and most preferably about 95% or more similarity to a polypeptide or amino acid sequence of BS106. This amino acid sequence can be selected from the group consisting of SEQUENCE ID NOS 20-33, and fragments thereof.

A “recombinant polypeptide,” “recombinant protein,” or “a polypeptide produced by recombinant techniques,” which terms may be used interchangeably herein, describes a polypeptide which by virtue of its origin or manipulation is not associated with all or a portion of the polypeptide with which it is associated in nature and/or is linked to a polypeptide other than that to which it is linked in nature. A recombinant or encoded polypeptide or protein is not necessarily translated from a designated nucleic acid sequence. It also may be generated in any manner, including chemical synthesis or expression of a recombinant expression system.

The term “syntheTic peptide” as used herein means a polymeric form of amino acids of any length, which may be chemically synthesized by methods well-known to the routineer. These synthetic peptides are useful in various applications.

The term “polynucleotide” as used herein means a polymeric form of nucleotides of any length, either ribonucleotides or deoxyribonucleotides. This term refers only to the primary structure of the molecule. Thus, the term includes double- and single-stranded DNA, as well as double- and single-stranded RNA. It also includes modifications, such as methylation or capping and unmodified forms of the polynucleotide. The terms “polynucleotide,” “oligomer,” “oligonucleotide,” and “oligo” are used interchangeably herein.

“A sequence corresponding to a cDNA” means that the sequence contains a polynucleotide sequence that is identical or complementary to a sequence in the designated DNA. The degree (or “percent”) of identity or complementarity to the cDNA will be approximately 50% or greater, preferably at least about 70% or greater, and more preferably at least about 90% or greater. The sequence that corresponds to the identified cDNA will be at least about 50 nucleotides in length, preferably at least about 60 nucleotides in length, and more preferably at least about 70 nucleotides in length. The correspondence between the gene or gene fragment of interest and the cDNA can be determined by methods known in the art and include, for example, a direct comparison of the sequenced material with the cDNAs described, or hybridization and digestion with single strand nucleases, followed by size determination of the digested fragments.

“Purified polynucleotide” refers to a polynucleotide of interest or fragment thereof which is essentially free, e.g., contains less than about 50%, preferably less than about 70%, and more preferably less than about 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

“Purified polypeptide” or “purified protein” means a polypeptide of interest or fragment thereof which is essentially free of, e.g., contains less than about 50%, preferably less than about 70%, and more preferably less than about 90%, cellular components with which the polypeptide of interest is naturally associated. Methods for purifying polypeptides of interest are known in the art.

The term “isolated” means that the material is removed from its original environment (e.g., the natural environment if it is naturally occurring). For example, a naturally-occurring polynucleotide or polypeptide present in a living animal is not isolated, but the same polynucleotide or DNA or polypeptide, which is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

“Polypeptide” and “protein” are used interchangeably herein and indicate at least one molecular chain of amino acids linked through covalent and/or non-covalent bonds. The terms do not refer to a specific length of the product. Thus peptides, oligopeptides and proteins are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

A “fragment” of a specified polypeptide refers to an amino acid sequence which comprises at least about 3-5 amino acids, more preferably at least about 8-10 amino acids, and even more preferably at least about 15-20 amino acids derived from the specified polypeptide.

“Recombinant host cells,” “host cells,” “cells,” “cell lines,” “cell cultures,” and other such terms denoting microorganisms or higher eukaryotic cell lines cultured as unicellular entities refer to cells which can be, or have been, used as recipients for recombinant vectors or other transferred DNA, and include the original progeny of the original cell which has been transfected.

As used herein “replicon” means any genetic element, such as a plasmid, a chromosome or a virus, that behaves as an autonomous unit of polynucleotide replication within a cell.

A “vector” is a replicon in which another polynucleotide segment is attached, such as to bring about the replication and/or expression of the attached segment.

The term “control sequence” refers to a polynucleotide sequence which is necessary to effect the expression of a coding sequence to which it is ligated. The nature of such control sequences differs depending upon the host organism. In prokaryotes, such control sequences generally include a promoter, a ribosomal binding site, and terminators; in eukaryotes, such control sequences generally include promoters, terminators and, in some instances, enhancers. The term “control sequence” thus is intended to include at a minimum all components whose presence is necessary for expression, and also may include additional components whose presence is advantageous, for example, leader sequences.

“Operably linked” refers to a situation wherein the components described are in a relationship permitting them to function in their intended manner. Thus, for example, a control sequence “operably linked” to a coding sequence is ligated in such a manner that expression of the coding sequence is achieved under conditions compatible with the control sequence.

The term “open reading frame” or “ORF” refers to a region of a polynucleotide sequence which encodes a polypeptide. This region may represent a portion of a coding sequence or a total coding sequence. Rare errors in translation may occur, termed translational frameshifting, or programmed frameshifting, that allow the ribosome to translate two partially overlapping reading frames as a single polypeptide I. P. Ivanov et al. RNA 4(10): 1230-1238 (1998); and P. J. Farabaugh Annu Rev Genet 30: 507-528 (1996).

A “coding sequence” is a polynucleotide sequence which is transcribed into mRNA and translated into a polypeptide when placed under the control of appropriate regulatory sequences. The boundaries of the coding sequence are determined by a translation start codon at the 5′-terminus and a translation stop codon at the 3′-terminus.

A coding sequence can include, but is not limited to, mRNA, cDNA and recombinant polynucleotide sequences.

The term “immunologically identifiable with/as” refers to the presence of epitope(s) and polypeptide(s) which also are present in and are unique to the designated polypeptide(s). Immunological identity may be determined by antibody binding and/or competition in binding. These techniques are known to the routineer and also are described herein. The uniqueness of an epitope also can be determined by computer searches of known data banks, such as GenBank, for the polynucleotide sequence which encodes the epitope and by amino acid sequence comparisons with other known proteins.

As used herein, “epitope” means an antigenic determinant of a polypeptide or protein. Conceivably, an epitope can comprise three amino acids in a spatial conformation which is unique to the epitope. Generally, an epitope consists of at least five such amino acids and more usually, it consists of at least eight to ten amino acids. Methods of examining spatial conformation are known in the art and include, for example, x-ray crystallography and two-dimensional nuclear magnetic resonance.

The methods for identifying epitopes in a novel peptide sequence are well known and described in both the scientific, commercial, and patent literature. For example, M. H. Van Regenmortel describes how to predict epitopes from the primary sequence of a protein. (See “Protein structure and antigenicity”, Int J Rad Appl Instrum B., 14(4): 277-80, 1987.)

Perkin-Elmer Biosystems, a major provider of DNA sequencing and peptide synthesizing instruments has established a public website which describes how to select peptides which reflect the epitopes of a protein. (See http://www.pebio.com/pa/340913/html/chapt2.html#Choosing the Epitope.) This electronic publication was posted in 1996 and basically describes the process employed by the inventors of the current patent application.

Patent application PCT/US97/00485 describes in detail how to identify epitopes from peptide sequences. The sequence can be scanned for hydrophobicity and hydrophilicity values by the method of Hopp, Prog. Clin. Biol. Res. 172B: 367-377 (1985) or the method of Cease et al, J. Exp. Med. 164: 1779-1784 (1986) or the method of Spouge et al, J. Immunol. 138: 204-212 (1987). Commercial software programs to implement these methods are available.

A “conformational epitope” is an epitope that is comprised of specific juxtaposition of amino acids in an immunologically recognizable structure, such amino acids being present on the same polypeptide in a contiguous or non-contiguous order or present on different polypeptides.

A polypeptide is “immunologically reactive” with an antibody when it binds to an antibody due to antibody recognition of a specific epitope contained within the polypeptide. Immunological reactivity may be determined by antibody binding, more particularly, by the kinetics of antibody binding, and/or by competition in binding using as competitor(s) a known polypeptide(s) containing an epitope against which the antibody is directed. The methods for determining whether a polypeptide is immunologically reactive with an antibody are known in the art.

As used herein, the term “immunogenic polypeptide containing an epitope of interest” means naturally occurring polypeptides of interest or fragments thereof, as well as polypeptides prepared by other means, for example, by chemical synthesis or the expression of the polypeptide in a recombinant organism.

The term “transfection” refers to the introduction of an exogenous polynucleotide into a prokaryotic or eucaryotic host cell, irrespective of the method used for the introduction. The term “transfection” refers to both stable and transient introduction of the polynucleotide, and encompasses direct uptake of polynucleotides, transformation, transduction, and f-mating. Once introduced into the host cell, the exogenous polynucleotide may be maintained as a non-integrated replicon, for example, a plasmid, or alternatively, may be integrated into the host genome.

“Treatment” refers to prophylaxis and/or therapy.

The term “individual” as used herein refers to vertebrates, particularly members of the mammalian species and includes, but is not limited to, domestic animals, sports animals, primates and humans; more particularly the term refers to humans.

The term “sense strand” or “plus strand” (or “+”) as used herein denotes a nucleic acid that contains the sequence that encodes the polypeptide. The term “antisense strand” or “minus strand” (or “−”) denotes a nucleic acid that contains a sequence that is complementary to that of the “plus” strand.

The term “test sample” refers to a component of an individual's body which is the source of the analyte (such as, antibodies of interest or antigens of interest). These components are well known in the art. A test sample is typically anything suspected of containing a target sequence. Test samples can be prepared using methodologies well known in the art such as by obtaining a specimen from an individual and, if necessary, disrupting any cells contained thereby to release target nucleic acids. These test samples include biological samples which can be tested by the methods of the present invention described herein and include human and animal body fluids such as whole blood, serum, plasma, cerebrospinal fluid, sputum, bronchial washing, bronchial aspirates, urine, lymph fluids and various external secretions of the respiratory, intestinal and genitourinary tracts, tears, saliva, milk, white blood cells, myelomas and the like; biological fluids such as cell culture supernatants; tissue specimens which may be fixed; and cell specimens which may be fixed.

“Purified product” refers to a preparation of the product which has been isolated from the cellular constituents with which the product is normally associated and from other types of cells which may be present in the sample of interest.

“PNA” denotes a “peptide nucleic acid analog” which may be utilized in a procedure such as an assay described herein to determine the presence of a target. “MA” denotes a “morpholino analog” which may be utilized in a procedure such as an assay described herein to determine the presence of a target. See, for example, U.S. Pat. No. 5,378,841, which is incorporated herein by reference. PNAs are neutrally charged moieties which can be directed against RNA targets or DNA. PNA probes used in assays in place of, for example, the DNA probes of the present invention, offer advantages not achievable when DNA probes are used. These advantages include manufacturability, large scale labeling, reproducibility, stability, insensitivity to changes in ionic strength and resistance to enzymatic degradation which is present in methods utilizing DNA or RNA. These PNAs can be labeled with (“attached to”) such signal generating compounds as fluorescein, radionucleotides, chemiluminescent compounds and the like. PNAs or other nucleic acid analogs such as MAs thus can be used in assay methods in place of DNA or RNA. Although assays are described herein utilizing DNA probes, it is within the scope of the routineer that PNAs or MAs can be substituted for RNA or DNA with appropriate changes if and as needed in assay reagents.

“Analyte,” as used herein, is the substance to be detected which may be present in the test sample. The analyte can be any substance for which there exists a naturally occurring specific binding member (such as, an antibody), or for which a specific binding member can be prepared. Thus, an analyte is a substance that can bind to one or more specific binding members in an assay. “Analyte” also includes any antigenic substances, haptens, antibodies and combinations thereof. As a member of a specific binding pair, the analyte can be detected by means of naturally occurring specific binding partners (pairs) such as the use of intrinsic factor protein as a member of a specific binding pair for the determination of Vitamin B12, the use of folate-binding protein to determine folic acid, or the use of a lectin as a member of a specific binding pair for the determination of a carbohydrate. The analyte can include a protein, a polypeptide, an amino acid, a nucleotide target and the like.

“Diseases of the breast” or “breast disease,” or “condition of the breast,” as used herein, refer to any disease or condition of the breast including, but not limited to, atypical hyperplasia, fibroadenoma, cystic breast disease, and cancer.

“Breast cancer,” as used herein, refers to any malignant disease of the breast including, but not limited to, ductal carcinoma in situ, lobular carcinoma in situ, infiltrating ductal carcinoma, medullary carcinoma, tubular carcinoma, mucinous carcinoma, infiltrating lobular carcinoma, infiltrating comedocarcinoma and inflammatory carcinoma.

An “Expressed Sequence Tag” or “EST” refers to the partial sequence of a cDNA insert which has been made by reverse transcription of mRNA extracted from a tissue followed by insertion into a vector.

A “transcript image” refers to a table or list giving the quantitative distribution of ESTs in a library and represents the genes active in the tissue from which the library was made.

The present invention provides assays which utilize specific binding members. A “specific binding member,” as used herein, is a member of a specific binding pair. That is, two different molecules where one of the molecules, through chemical or physical means, specifically binds to the second molecule. Therefore, in addition to antigen and antibody specific binding pairs of common immunoassays, other specific binding pairs can include biotin and avidin, carbohydrates and lectins, complementary nucleotide sequences, effector and receptor molecules, cofactors and enzymes, enzyme inhibitors and enzymes and the like. Furthermore, specific binding pairs can include members that are analogs of the original specific binding members, for example, an analyte-analog. Immunoreactive specific binding members include antigens, antigen fragments, antibodies and antibody fragments, both monoclonal and polyclonal and complexes thereof, including those formed by recombinant DNA molecules.

Specific binding members include “specific binding molecules.” A “specific binding molecule” intends any specific binding member, particularly an immunoreactive specific binding member. As such, the term “specific binding molecule” encompasses antibody molecules (obtained from both polyclonal and monoclonal preparations), as well as, the following: hybrid (chimeric) antibody molecules (see, for example, Winter, et al., Nature 349: 293-299 (1991), and U.S. Pat. No. 4,816,567); F(ab′)₂ and F(ab) fragments; Fv molecules (non-covalent heterodimers, see, for example, Inbar, et al., Proc. Natl. Acad. Sci. USA 69: 2659-2662 (1972), and Ehrlich, et al., Biochem. 19: 4091-4096 (1980)); single chain Fv molecules (sFv) (see, for example, Huston, et al., Proc. Natl. Acad. Sci. USA 85: 5879-5883 (1988)); humanized antibody molecules (see, for example, Riechmann, et al., Nature 332: 323-327 (1988), Verhoeyan, et al., Science 239: 1534-1536 (1988), and UK Patent Publication No. GB 2,276,169, published 21 Sep. 1994); and, any functional fragments obtained from such molecules, wherein such fragments retain immunological binding properties of the parent antibody molecule.

The term “hapten,” as used herein, refers to a partial antigen or non-protein binding member which is capable of binding to an antibody, but which is not capable of eliciting antibody formation unless coupled to a carrier protein.

A “capture reagent,” as used herein, refers to an unlabeled specific binding member which is specific either for the analyte as in a sandwich assay, for the indicator reagent or analyte as in a competitive assay, or for an ancillary specific binding member, which itself is specific for the analyte, as in an indirect assay. The capture reagent can be directly or indirectly bound to a solid phase material before the performance of the assay or during the performance of the assay, thereby enabling the separation of immobilized complexes from the test sample.

The “indicator reagent” comprises a “signal-generating compound” (“label”) which is capable of generating and generates a measurable signal detectable by external means, conjugated (“attached”) to a specific binding member. In addition to being an antibody member of a specific binding pair, the indicator reagent also can be a member of any specific binding pair, including either hapten-anti-hapten systems such as biotin or anti-biotin, avidin or biotin, a carbohydrate or a lectin, a complementary nucleotide sequence, an effector or a receptor molecule, an enzyme cofactor and an enzyme, an enzyme inhibitor or an enzyme and the like. An immunoreactive specific binding member can be an antibody, an antigen, or an antibody/antigen complex that is capable of binding either to the polypeptide of interest as in a sandwich assay, to the capture reagent as in a competitive assay, or to the ancillary specific binding member as in an indirect assay. When describing probes and probe assays, the term “reporter molecule” may be used. A reporter molecule comprises a signal generating compound as described hereinabove conjugated to a specific binding member of a specific binding pair, such as carbazole or adamantane.

The various “signal-generating compounds” (labels) contemplated include chromagens, catalysts such as enzymes, luminescent compounds such as fluorescein and rhodamine, chemiluminescent compounds such as dioxetanes, acridiniums, phenanthridiniums and luminol, radioactive elements and direct visual labels. Examples of enzymes include alkaline phosphatase, horseradish peroxidase, beta-galactosidase and the like. The selection of a particular label is not critical, but it must be capable of producing a signal either by itself or in conjunction with one or more additional substances.

“Solid phases” (“solid supports”) are known to those in the art and include the walls of wells of a reaction tray, test tubes, polystyrene beads, magnetic or non-magnetic beads, nitrocellulose strips, membranes, microparticles such as latex particles, sheep (or other animal) red blood cells and Duracytes® (red blood cells “fixed” by pyruvic aldehyde and formaldehyde, available from Abbott Laboratories, Abbott Park, Ill.) and others. The “solid phase” is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and Duracytes® are all suitable examples. Suitable methods for immobilizing peptides on solid phases include ionic, hydrophobic, covalent interactions and the like. A “solid phase,” as used herein, refers to any material which is insoluble, or can be made insoluble by a subsequent reaction. The solid phase can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid phase and which has the ability to immobilize the capture reagent through a specific binding reaction. The receptor molecule enables the indirect binding of the capture reagent to a solid phase material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, Duracytes® and other configurations known to those of ordinary skill in the art.

It is contemplated and within the scope of the present invention that the solid phase also can comprise any suitable porous material with sufficient porosity to allow access by detection antibodies and a suitable surface affinity to bind antigens. Microporous structures generally are preferred, but materials with a gel structure in the hydrated state may be used as well. Such useful solid supports include, but are not limited to, nitrocellulose and nylon. It is contemplated that such porous solid supports described herein preferably are in the form of sheets of thickness from about 0.01 to 0.5 mm, preferably about 0.1 mm. The pore size may vary within wide limits and preferably is from about 0.025 to 15 microns, especially from about 0.15 to 15 microns. The surface of such supports may be activated by chemical processes which cause covalent linkage of the antigen or antibody to the support. The irreversible binding of the antigen or antibody is obtained, however, in general, by adsorption on the porous material by poorly understood hydrophobic forces. Other suitable solid supports are known in the art.

Reagents

The present invention provides reagents such as polynucleotide sequences derived from a breast tissue of interest and designated as BS106, polypeptides encoded thereby and antibodies specific for these polypeptides. The present invention also provides reagents such as oligonucleotide fragments derived from the disclosed polynucleotides and nucleic acid sequences complementary to these polynucleotides. The polynucleotides, polypeptides, or antibodies of the present invention may be used to provide information leading to the detecting, diagnosing, staging, monitoring, prognosticating, preventing or treating of, or determining the predisposition to, diseases and conditions of the breast such as cancer. The sequences disclosed herein represent unique polynucleotides which can be used in assays or for producing a specific profile of gene transcription activity. Such assays are disclosed in European Patent Number 0373203B1 and International Publication No. WO 95/11995, which are hereby incorporated by reference.

Selected BS106-derived polynucleotides can be used in the methods described herein for the detection of normal or altered gene expression. Such methods may employ BS106 polynucleotides or oligonucleotides, fragments or derivatives thereof, or nucleic acid sequences complementary thereto.

The polynucleotides disclosed herein, their complementary sequences, or fragments of either, can be used in assays to detect, amplify or quantify genes, nucleic acids, cDNAs or mRNAs relating to breast tissue disease and conditions associated therewith. They also can be used to identify an entire or partial coding region of a BS106 polypeptide. They further can be provided in individual containers in the form of a kit for assays, or provided as individual compositions. If provided in a kit for assays, other suitable reagents such as buffers, conjugates and the like may be included.

The polynucleotide may be in the form of RNA or DNA. Polynucleotides in the form of DNA, cDNA, genomic DNA, nucleic acid analogs and synthetic DNA are within the scope of the present invention. The DNA may be double-stranded or single-stranded, and if single stranded, may be the coding (sense) strand or non-coding (anti-sense) strand. The coding sequence which encodes the polypeptide may be identical to the coding sequence provided herein or may be a different coding sequence which coding sequence, as a result of the redundancy or degeneracy of the genetic code, encodes the same polypeptide as the DNA provided herein.

This polynucleotide may include only the coding sequence for the polypeptide, or the coding sequence for the polypeptide and an additional coding sequence such as a leader or secretory sequence or a proprotein sequence, or the coding sequence for the polypeptide (and optionally an additional coding sequence) and a non-coding sequence, such as a non-coding sequence 5′ and/or 3′ of the coding sequence for the polypeptide.

In addition, the invention includes variant polynucleotides containing modifications such as polynucleotide deletions, substitutions or additions; and any polypeptide modification resulting from the variant polynucleotide sequence. A polynucleotide of the present invention also may have a coding sequence which is a naturally occurring allelic variant of the coding sequence provided herein.

In addition, the coding sequence for the polypeptide may be fused in the same reading frame to a polynucleotide sequence which aids in expression and secretion of a polypeptide from a host cell, for example, a leader sequence which functions as a secretory sequence for controlling transport of a polypeptide from the cell. The polypeptide having a leader sequence is a preprotein and may have the leader sequence cleaved by the host cell to form the polypeptide. The polynucleotides may also encode for a proprotein which is the protein plus additional 5′ amino acid residues. A protein having a prosequence is a proprotein and may, in some cases, be an inactive form of the protein. Once the prosequence is cleaved an active protein remains. Thus, the polynucleotide of the present invention may encode for a protein, or for a protein having a prosequence, or for a protein having both a presequence (leader sequence) and a prosequence.

The polynucleotides of the present invention may also have the coding sequence fused in frame to a marker sequence which allows for purification of the polypeptide of the present invention. The marker sequence may be a hexa-histidine tag supplied by a pQE-9 vector to provide for purification of the polypeptide fused to the marker in the case of a bacterial host, or, for example, the marker sequence may be a hemagglutinin (HA) tag when a mammalian host, e.g. a COS-7 cell line, is used. The HA tag corresponds to an epitope derived from the influenza hemagglutinin protein. See, for example, I. Wilson et al., Cell 37: 767 (1984).

It is contemplated that polynucleotides will be considered to hybridize to the sequences provided herein if there is at least 50%, preferably at least 70%, and more preferably at least 90% identity between the polynucleotide and the sequence.

The degree of sequence identity between two nucleic acid molecules greatly affects the efficiency and strength of hybridization events between such molecules. A partially identical nucleic acid sequence is one that will at least partially inhibit a completely identical sequence from hybridizing to a target molecule. Inhibition of hybridization of the completely identical sequence can be assessed using hybridization assays that are well known in the art (e.g., Southern blot, Northern blot, solution hybridization, in situ hybridization, or the like, see Sambrook, et. al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. In one embodiment of the present invention, a nucleic acid molecule is capable of hybridizing selectively to a target sequence under moderately stringent hybridization conditions. In the context of the present invention, moderately stringent hybridization conditions allow detection of a target nucleic acid sequence of at least 14 nucleotides in length having at least approximately 70% sequence identity with the sequence of the selected nucleic acid probe. In another embodiment, such selective hybridization is performed under stringent hybridization conditions. Stringent hybridization conditions allow detection of target nucleic acid sequences of at least 14 nucleotides in length having a sequence identity of greater than 90% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Hybrid molecules can be formed, for example, on a solid support, in solution, and in tissue sections. The formation of hybrids can be monitored by inclusion of a reporter molecule, typically, in the probe. Such reporter molecules, or detectable elements include, but are not limited to, radioactive elements, fluorescent markers, and molecules to which an enzyme-conjugated ligand can bind.

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is well within the skill of the routineer in the art (see, for example, Sambrook, et al., Molecular Cloning: A Laboratory Manual, Second Edition, (1989) Cold Spring Harbor, N.Y.).

The present invention also provides an antibody produced by using a purified BS106 polypeptide of which at least a portion of the polypeptide is encoded by a BS106 polynucleotide selected from the polynucleotides provided herein. These antibodies may be used in the methods provided herein for the detection of BS106 antigen in test samples. The presence of BS106 antigen in the test samples is indicative of the presence of a breast disease or condition. The antibody also may be used for therapeutic purposes, for example, in neutralizing the activity of BS106 polypeptide in conditions associated with altered or abnormal expression.

The present invention further relates to a BS106 polypeptide which has the deduced amino acid sequence as provided herein, as well as fragments, analogs and derivatives of such polypeptide. The polypeptide of the present invention may be a recombinant polypeptide, a natural purified polypeptide or a synthetic polypeptide. The fragment, derivative or analog of the BS106 polypeptide may be one in which one or more of the amino acid residues is substituted with a conserved or non-conserved amino acid residue (preferably a conserved amino acid residue) and such substituted amino acid residue may or may not be one encoded by the genetic code; or it may be one in which one or more of the amino acid residues includes a substituent group; or it may be one in which the polypeptide is fused with another compound, such as a compound to increase the half-life of the polypeptide (for example, polyethylene glycol); or it may be one in which the additional amino acids are fused to the polypeptide, such as a leader or secretory sequence or a sequence which is employed for purification of the polypeptide or a proprotein sequence. Such fragments, derivatives and analogs are within the scope of the present invention. The polypeptides and polynucleotides of the present invention are provided preferably in an isolated form and preferably purified.

Thus, a polypeptide of the present invention may have an amino acid sequence that is identical to that of the naturally occurring polypeptide or that is different by minor variations due to one or more amino acid substitutions. The variation may be a “conservative change” typically in the range of about 1 to 5 amino acids, wherein the substituted amino acid has similar structural or chemical properties, e.g., replacement of leucine with isoleucine or threonine with serine. In contrast, variations may include nonconservative changes, e.g., replacement of a glycine with a tryptophan. Similar minor variations may also include amino acid deletions or insertions, or both. Guidance in determining which and how many amino acid residues may be substituted, inserted or deleted without changing biological or immunological activity may be found using computer programs well known in the art, for example, DNASTAR software (DNASTAR Inc., Madison Wis.).

Probes constructed according to the polynucleotide sequences of the present invention can be used in various assay methods to provide various types of analysis. For example, such probes can be used in fluorescent in situ hybridization (FISH) technology to perform chromosomal analysis, and used to identify cancer-specific structural alterations in the chromosomes, such as deletions or translocations that are visible from chromosome spreads or detectable using PCR-generated and/or allele specific oligonucleotides probes, allele specific amplification or by direct sequencing. Probes also can be labeled with radioisotopes, directly- or indirectly-detectable haptens, or fluorescent molecules, and utilized for in situ hybridization studies to evaluate the mRNA expression of the gene comprising the polynucleotide in tissue specimens or cells.

This invention also provides teachings as to the production of the polynucleotides and polypeptides provided herein.

Probe Assays

The sequences provided herein may be used to produce probes which can be used in assays for the detection of nucleic acids in test samples. The probes may be designed from conserved nucleotide regions of the polynucleotides of interest or from non-conserved nucleotide regions of the polynucleotide of interest. The design of such probes for optimization in assays is within the skill of the routineer. Generally, nucleic acid probes are developed from non-conserved or unique regions when maximum specificity is desired, and nucleic acid probes are developed from conserved regions when assaying for nucleotide regions that are closely related to, for example, different members of a multi-gene family or in related species like mouse and man.

The polymerase chain reaction (PCR) is a technique for amplifying a desired nucleic acid sequence (target) contained in a nucleic acid or mixture thereof. In PCR, a pair of primers are employed in excess to hybridize to the complementary strands of the target nucleic acid. The primers are each extended by a polymerase using the target nucleic acid as a template. The extension products become target sequences themselves, following dissociation from the original target strand. New primers then are hybridized and extended by a polymerase, and the cycle is repeated to geometrically increase the number of target sequence molecules. PCR is disclosed in U.S. Pat. Nos. 4,683,195 and 4,683,202, which are incorporated herein by reference.

The Ligase Chain Reaction (LCR) is an alternate method for nucleic acid amplification. In LCR, probe pairs are used which include two primary (first and second) and two secondary (third and fourth) probes, all of which are employed in molar excess to target. The first probe hybridizes to a first segment of the target strand, and the second probe hybridizes to a second segment of the target strand, the first and second segments being contiguous so that the primary probes abut one another in 5′ phosphate-3′ hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. Of course, if the target is initially double stranded, the secondary probes also will hybridize to the target complement in the first instance. Once the ligated strand of primary probes is separated from the target strand, it will hybridize with the third and fourth probes which can be ligated to form a complementary, secondary ligated product. It is important to realize that the ligated products are functionally equivalent to either the target or its complement. By repeated cycles of hybridization and ligation, amplification of the target sequence is achieved. This technique is described more completely in EP-A-320 308 to K. Backman published Jun. 16, 1989 and EP-A-439 182 to K. Backman et al, published Jul. 31, 1991, both of which are incorporated herein by reference.

For amplification of mRNAs, it is within the scope of the present invention to reverse transcribe mRNA into cDNA followed by polymerase chain reaction (RT-PCR);
or, to use a single enzyme for both steps as described in U.S. Pat. No. 5,322,770, which is incorporated herein by reference; or reverse transcribe mRNA into cDNA followed by asymmetric gap ligase chain reaction (RT-AGLCR) as described by R. L. Marshall et al., PCR Methods and Applications 4: 80-84 (1994), which also is incorporated herein by reference.

Other known amplification methods which can be utilized herein include but are not limited to the so-called “NASBA” or “3 SR” technique described by J. C. Guatelli, et al., PNAS USA 87: 1874-1878 (1990) and also described by J. Compton, Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996)) and European Patent Application No. 684315; and target mediated amplification, as described in International Publication No. WO 93/22461.

Detection of BS106 may be accomplished using any suitable detection method, including those detection methods which are currently well known in the art, as well as detection strategies which may evolve later. Examples of the foregoing presently known detection methods are hereby incorporated herein by reference. See, for example, Caskey et al., U.S. Pat. No. 5,582,989, Gelfand et al., U.S. Pat. No. 5,210,015. Examples of such detection methods include target amplification methods as well as signal amplification technologies. An example of presently known detection methods would include the nucleic acid amplification technologies referred to as PCR, LCR, NASBA, SDA, RCR and TMA. See, for example, Caskey et al., U.S. Pat. No. 5,582,989, Gelfand et al., U.S. Pat. No. 5,210,015. All of the foregoing are hereby incorporated by reference. Detection may also be accomplished using signal amplification such as that disclosed in Snitman et al., U.S. Pat. No. 5,273,882. While the amplification of target or signal is preferred at present, it is contemplated and within the scope of the present invention that ultrasensitive detection methods which do not require amplification can be utilized herein.

Detection, both amplified and non-amplified, may be (combined) carried out using a variety of heterogeneous and homogeneous detection formats. Examples of heterogeneous detection formats are disclosed in Snitman et al., U.S. Pat. No. 5,273,882, Albarella et al in EP-84114441.9, Urdea et al., U.S. Pat. No. 5,124,246, Ullman et al. U.S. Pat. No. 5,185,243 and Kourilsky et al., U.S. Pat. No. 4,581,333. All of the foregoing are hereby incorporated by reference. Examples of homogeneous detection formats are disclosed in, Caskey et al., U.S. Pat. No. 5,582,989, Gelfand et al., U.S. Pat. No. 5,210,015, which are incorporated herein by reference. Also contemplated and within the scope of the present invention is the use of multiple probes in the hybridization assay, which use improves sensitivity and amplification of the BS106 signal. See, for example, Caskey et al., U.S. Pat. No. 5,582,989, Gelfand et al., U.S. Pat. No. 5,210,015, which are incorporated herein by reference.

In one embodiment, the present invention generally comprises the steps of contacting a test sample suspected of containing a target polynucleotide sequence with amplification reaction reagents comprising an amplification primer, and a detection probe that can hybridize with an internal region of the amplicon sequences. Probes and primers employed according to the method provided herein are labeled with capture and detection labels, wherein probes are labeled with one type of label and primers are labeled with another type of label. Additionally, the primers and probes are selected such that the probe sequence has a lower melt temperature than the primer sequences. The amplification reagents, detection reagents and test sample are placed under amplification conditions whereby, in the presence of target sequence, copies of the target sequence (an amplicon) are produced. In the usual case, the amplicon is double stranded because primers are provided to amplify a target sequence and its complementary strand. The double stranded amplicon then is thermally denatured to produce single stranded amplicon members. Upon formation of the single stranded amplicon members, the mixture is cooled to allow the formation of complexes between the probes and single stranded amplicon members.

As the single stranded amplicon sequences and probe sequences are cooled, the probe sequences preferentially bind the single stranded amplicon members. This finding is counterintuitive given that the probe sequences generally are selected to be shorter than the primer sequences and therefore have a lower melt temperature than the primers. Accordingly, the melt temperature of the amplicon produced by the primers should also have a higher melt temperature than the probes. Thus, as the mixture cools, the re-formation of the double stranded amplicon would be expected. As previously stated, however, this is not the case. The probes are found to preferentially bind the single stranded amplicon members. Moreover, this preference of probe/single stranded amplicon binding exists even when the primer sequences are added in excess of the probes.

After the probe/single stranded amplicon member hybrids are formed, they are detected. Standard heterogeneous assay formats are suitable for detecting the hybrids using the detection labels and capture labels present on the primers and probes. The hybrids can be bound to a solid phase reagent by virtue of the capture label and detected by virtue of the detection label. In cases where the detection label is directly detectable, the presence of the hybrids on the solid phase can be detected by causing the label to produce a detectable signal, if necessary, and detecting the signal. In cases where the label is not directly detectable, the captured hybrids can be contacted with a conjugate, which generally comprises a binding member attached to a directly detectable label. The conjugate becomes bound to the complexes and the conjugates presence on the complexes can be detected with the directly detectable label. Thus, the presence of the hybrids on the solid phase reagent can be determined. Those skilled in the art will recognize that wash steps may be employed to wash away unhybridized amplicon or probe as well as unbound conjugate.

In one embodiment, the heterogeneous assays can be conveniently performed using a solid phase support that carries an array of nucleic acid molecules. Such arrays are useful for high-throughput and/or multiplexed assay formats. Various methods for forming such arrays from pre-formed nucleic acid molecules, or methods for generating the array using in situ synthesis techniques, are generally known in the art. (See, for example, Dattagupta, et al., EP Publication No. 0 234, 726A3; Southern, U.S. Pat. No. 5,700,637; Pirrung, et al., U.S. Pat. No. 5,143,854; PCT International Publication No. WO 92/10092; and, Fodor, et al., Science 251: 767-777 (1991)).

Although the target sequence is described as single stranded, it also is contemplated to include the case where the target sequence is actually double stranded but is merely separated from its complement prior to hybridization with the amplification primer sequences. In the case where PCR is employed in this method, the ends of the target sequences are usually known. In cases where LCR or a modification thereof is employed in the preferred method, the entire target sequence is usually known. Typically, the target sequence is a nucleic acid sequence such as, for example, RNA or DNA.

The method provided herein can be used in well-known amplification reactions that include thermal cycle reaction mixtures, particularly in PCR and gap LCR (GLCR). Amplification reactions typically employ primers to repeatedly generate copies of a target nucleic acid sequence, which target sequence is usually a small region of a much larger nucleic acid sequence. Primers are themselves nucleic acid sequences that are complementary to regions of a target sequence. Under amplification conditions, these primers hybridize or bind to the complementary regions of the target sequence. Copies of the target sequence typically are generated by the process of primer extension and/or ligation which utilizes enzymes with polymerase or ligase activity, separately or in combination, to add nucleotides to the hybridized primers and/or ligate adjacent probe pairs. The nucleotides that are added to the primers or probes, as monomers or preformed oligomers, are also complementary to the target sequence. Once the primers or probes have been sufficiently extended and/or ligated, they are separated from the target sequence, for example, by heating the reaction mixture to a “melt temperature” which is one in which complementary nucleic acid strands dissociate. Thus, a sequence complementary to the target sequence is formed.

A new amplification cycle then can take place to further amplify the number of target sequences by separating any double stranded sequences, allowing primers or probes to hybridize to their respective targets, extending and/or ligating the hybridized primers or probes and re-separating. The complementary sequences that are generated by amplification cycles can serve as templates for primer extension or filling the gap of two probes to further amplify the number of target sequences. Typically, a reaction mixture is cycled between 20 and 100 times, more typically, a reaction mixture is cycled between 25 and 50 times. The numbers of cycles can be determined by the routineer. In this manner, multiple copies of the target sequence and its complementary sequence are produced. Thus, primers initiate amplification of the target sequence when it is present under amplification conditions.

Generally, two primers which are complementary to a portion of a target strand and its complement are employed in PCR. For LCR, four probes, two of which are complementary to a target sequence and two of which are similarly complementary to the target's complement, are generally employed. In addition to the primer sets and enzymes previously mentioned, a nucleic acid amplification reaction mixture may also comprise other reagents which are well known and include but are not limited to: enzyme cofactors such as manganese; magnesium; salts; nicotinamide adenine dinucleotide (NAD); and deoxynucleotide triphosphates (dNTPs) such, as for example, deoxyadenine triphosphate, deoxyguanine triphosphate, deoxycytosine triphosphate and deoxythymine triphosphate.

While the amplification primers initiate amplification of the target sequence, the detection (or hybridization) probe is not involved in amplification. Detection probes are generally nucleic acid sequences or uncharged nucleic acid analogs such as, for example, peptide nucleic acids which are disclosed in International Publication No. WO 92/20702; morpholino analogs which are described in U.S. Pat. Nos. 5,185,444, 5,034,506 and 5,142,047; and the like. Depending upon the type of label carried by the probe, the probe is employed to capture or detect the amplicon generated by the amplification reaction. The probe is not involved in amplification of the target sequence and therefore may have to be rendered “non-extendible” in that additional dNTPs cannot be added to the probe. In and of themselves analogs usually are non-extendible and nucleic acid probes can be rendered non-extendible by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified. U.S. patent application Ser. No. 07/049,061 filed Apr. 19, 1993 and incorporated herein by reference describes modifications which can be used to render a probe non-extendible.

The ratio of primers to probes is not important. Thus, either the probes or primers can be added to the reaction mixture in excess whereby the concentration of one would be greater than the concentration of the other. Alternatively, primers and probes can be employed in equivalent concentrations. Preferably, however, the primers are added to the reaction mixture in excess of the probes. Thus, primer to probe ratios of, for example, 5:1 and 20:1 are preferred.

While the length of the primers and probes can vary, the probe sequences are selected such that they have a lower melt temperature than the primer sequences. Hence, the primer sequences are generally longer than the probe sequences. Typically, the primer sequences are in the range of between 20 and 50 nucleotides long, more typically in the range of between 20 and 30 nucleotides long. The typical probe is in the range of between 10 and 25 nucleotides long.

Various methods for synthesizing primers and probes are well known in the art. Similarly, methods for attaching labels to primers or probes are also well known in the art. For example, it is a matter of routine to synthesize desired nucleic acid primers or probes using conventional nucleotide phosphoramidite chemistry and instruments available from Applied Biosystems, Inc., (Foster City, Calif.), DuPont (Wilmington, Del.), or Milligen (Bedford Mass.). Many methods have been described for labeling oligonucleotides such as the primers or probes of the present invention. Enzo Biochemical (New York, N.Y.) and Clontech (Palo Alto, Calif.) both have described and commercialized probe labeling techniques. For example, a primary amine can be attached to a 3′ oligo terminus using 3′-Amine-ON CPG™ (Clontech, Palo Alto, Calif.). Similarly, a primary amine can be attached to a 5′ oligo terminus using Aminomodifier II® (Clontech). The amines can be reacted to various haptens using conventional activation and linking chemistries. In addition, copending applications U.S. Serial Nos. 625,566, filed Dec. 11, 1990 and 630,908, filed Dec. 20, 1990, which are each incorporated herein by reference, teach methods for labeling probes at their 5′ and 3′ termini, respectively. International Publication Nos WO 92/10505, published 25 Jun. 1992, and WO 92/11388, published 9 Jul. 1992, teach methods for labeling probes at their 5′ and 3′ ends, respectively. According to one known method for labeling an oligonucleotide, a label-phosphoramidite reagent is prepared and used to add the label to the oligonucleotide during its synthesis. See, for example, N. T. Thuong et al., Tet. Letters 29(46): 5905-5908 (1988); or J. S. Cohen et al., published U.S. patent application Ser. No. 07/246,688 (NTIS ORDER No. PAT-APPL-7-246,688) (1989). Preferably, probes are labeled at their 3′ and 5′ ends.

A capture label is attached to the primers or probes and can be a specific binding member which forms a binding pair with the solid phase reagent's specific binding member. It will be understood that the primer or probe itself may serve as the capture label. For example, in the case where a solid phase reagent's binding member is a nucleic acid sequence, it may be selected such that it binds a complementary portion of the primer or probe to thereby immobilize the primer or probe to the solid phase. In cases where the probe itself serves as the binding member, those skilled in the art will recognize that the probe will contain a sequence or “tail” that is not complementary to the single stranded amplicon members. In the case where the primer itself serves as the capture label, at least a portion of the primer will be free to hybridize with a nucleic acid on a solid phase because the probe is selected such that it is not fully complementary to the primer sequence.

Generally, probe/single stranded amplicon member complexes can be detected using techniques commonly employed to perform heterogeneous immunoassays. Preferably, in this embodiment, detection is performed according to the protocols used by the commercially available Abbott LCx® instrumentation (Abbott Laboratories, Abbott Park, Ill.).

The primers and probes disclosed herein are useful in typical PCR assays, wherein the test sample is contacted with a pair of primers, amplification is performed, the hybridization probe is added, and detection is performed.

Another method provided by the present invention comprises contacting a test sample with a plurality of polynucleotides, wherein at least one polynucleotide is a BS106 molecule as described herein, hybridizing the test sample with the plurality of polynucleotides and detecting hybridization complexes. Hybridization complexes are identified and quantitated to compile a profile which is indicative of breast tissue disease, such as breast cancer. Expressed RNA sequences may further be detected by reverse transcription and amplification of the DNA product by procedures well-known in the art, including polymerase chain reaction (PCR).

Drug Screening and Gene Therapy.

The present invention also encompasses the use of gene therapy methods for the introduction of anti-sense BS106 derived molecules, such as polynucleotides or oligonucleotides of the present invention, into patients with conditions associated with abnormal expression of polynucleotides related to a breast tissue disease or condition especially breast cancer. These molecules, including antisense RNA and DNA fragments and ribozymes, are designed to inhibit the translation of BS106-mRNA, and may be used therapeutically in the treatment of conditions associated with altered or abnormal expression of a BS106 polynucleotide.

Alternatively, the oligonucleotides described above can be delivered to cells by procedures known in the art such that the anti-sense RNA or DNA may be expressed in vivo to inhibit production of a BS106 polypeptide in the manner described above. Antisense constructs to a BS106 polynucleotide, therefore, reverse the action of BS106 transcripts and may be used for treating breast tissue disease conditions, such as breast cancer. These antisense constructs may also be used to treat tumor metastases.

The present invention also provides a method of screening a plurality of compounds for specific binding to BS106 polypeptide(s), or any fragment thereof, to identify at least one compound which specifically binds the BS106 polypeptide. Such a method comprises the steps of providing at least one compound; combining the BS106 polypeptide with each compound under suitable conditions for a time sufficient to allow binding; and detecting the BS106 polypeptide binding to each compound.

The polypeptide or peptide fragment employed in such a test may either be free in solution, affixed to a solid support, borne on a cell surface or located intracellularly. One method of screening utilizes eukaryotic or prokaryotic host cells which are stably transfected with recombinant nucleic acids which can express the polypeptide or peptide fragment. A drug, compound, or any other agent, may be screened against such transfected cells in competitive binding assays. For example, the formation of complexes between a polypeptide and the agent being tested can be measured in either viable or fixed cells.

The present invention thus provides methods of screening for drugs, compounds, or any other agent, which can be used to treat diseases associated with BS106. These methods comprise contacting the agent with a polypeptide or fragment thereof and assaying for either the presence of a complex between the agent and the polypeptide, or for the presence of a complex between the polypeptide and the cell. In competitive binding assays, the polypeptide typically is labeled. After suitable incubation, free (or uncomplexed) polypeptide or fragment thereof is separated from that present in bound form, and the amount of free or uncomplexed label is used as a measure of the ability of the particular agent to bind to the polypeptide or to interfere with the polypeptide/cell complex.

The present invention also encompasses the use of competitive screening assays in which neutralizing antibodies capable of binding polypeptide specifically compete with a test agent for binding to the polypeptide or fragment thereof. In this manner, the antibodies can be used to detect the presence of any polypeptide in the test sample which shares one or more antigenic determinants with a BS106 polypeptide as provided herein.

Another technique for agent screening provides high throughput screening for compounds having suitable binding affinity to at least one polypeptide of BS106 disclosed herein. Briefly, large numbers of different small peptide test compounds are synthesized on a solid phase, such as plastic pins or some other surface. The peptide test compounds are reacted with polypeptide and washed. Polypeptide thus bound to the solid phase is detected by methods well-known in the art. Purified polypeptide can also be coated directly onto plates for use in the screening techniques described herein. In addition, non-neutralizing antibodies can be used to capture the polypeptide and immobilize it on the solid support. See, for example, EP 84/03564, published on Sep. 13, 1984, which is incorporated herein by reference.

The goal of rational drug design is to produce structural analogs of biologically active polypeptides of interest or of the small molecules including agonists, antagonists, or inhibitors with which they interact. Such structural analogs can be used to design drugs which are more active or stable forms of the polypeptide or which enhance or interfere with the function of a polypeptide in vivo. J. Hodgson, Bio/Technology 9: 19-21 (1991), incorporated herein by reference.

For example, in one approach, the three-dimensional structure of a polypeptide, or of a polypeptide-inhibitor complex, is determined by x-ray crystallography, by computer modeling or, most typically, by a combination of the two approaches. Both the shape and charges of the polypeptide must be ascertained to elucidate the structure and to determine active site(s) of the molecule. Less often, useful information regarding the structure of a polypeptide may be gained by modeling based on the structure of homologous proteins. In both cases, relevant structural information is used to design analogous polypeptide-like molecules or to identify efficient inhibitors.

Useful examples of rational drug design may include molecules which have improved activity or stability as shown by S. Braxton et al., Biochemistry 31: 7796-7801 (1992), or which act as inhibitors, agonists, or antagonists of native peptides as shown by S. B. P. Athauda et al., J. Biochem. (Tokyo) 113 (6): 742-746 (1993), incorporated herein by reference.

It also is possible to isolate a target-specific antibody selected by an assay as described hereinabove, and then to determine its crystal structure. In principle, this approach yields a pharmacophore upon which subsequent drug design can be based. It further is possible to bypass protein crystallography altogether by generating anti-idiotypic antibodies (“anti-ids”) to a functional, pharmacologically active antibody. As a mirror image of a mirror image, the binding site of the anti-id is an analog of the original receptor. The anti-id then can be used to identify and isolate peptides from banks of chemically or biologically produced peptides. The isolated peptides then can act as the pharmacophore (that is, a prototype pharmaceutical drug).

A sufficient amount of a recombinant polypeptide of the present invention may be made available to perform analytical studies such as X-ray crystallography. In addition, knowledge of the polypeptide amino acid sequence which is derivable from the nucleic acid sequence provided herein will provide guidance to those employing computer modeling techniques in place of, or in addition to, x-ray crystallography.

Antibodies specific to a BS106 polypeptide (e.g., anti-BS106 antibodies) further may be used to inhibit the biological action of the polypeptide by binding to the polypeptide. In this manner, the antibodies may be used in therapy, for example, to treat breast tissue diseases including breast cancer and its metastases.

Further, such antibodies can detect the presence or absence of the BS106 polypeptide in a test sample and, therefore, are useful as diagnostic markers for the diagnosis of a breast tissue disease or condition especially breast cancer. Such antibodies may also function as a diagnostic marker for breast tissue disease conditions such as breast cancer.

The present invention also is directed to antagonists and inhibitors of the polypeptides of the present invention. The antagonists and inhibitors are those which inhibit or eliminate the function of the polypeptide. Thus, for example, an antagonist may bind to a polypeptide of the present invention and inhibit or eliminate its function. The antagonist, for example, could be an antibody against the polypeptide which eliminates the activity of the BS106 polypeptide by binding the BS106 polypeptide, or in some cases the antagonist may be an oligonucleotide. Examples of small molecule inhibitors include, but are not limited to, small peptides or peptide-like molecules.

The antagonists and inhibitors may be employed as a composition with a pharmaceutically acceptable carrier, including, but not limited to, saline, buffered saline, dextrose, water, glycerol, ethanol and combinations thereof. Administration of BS106 polypeptide inhibitors is preferably systemic. The present invention also provides an antibody which inhibits the action of such a polypeptide.

Antisense technology can be used to reduce gene expression through triple-helix formation or antisense DNA or RNA, both of which methods are based on binding of a polynucleotide to DNA or RNA. For example, the 5′ coding portion of the polynucleotide sequence, which encodes for the polypeptide of the present invention, is used to design an antisense RNA oligonucleotide of from 10 to 40 base pairs in length. A DNA oligonucleotide is designed to be complementary to a region of the gene involved in transcription, thereby preventing transcription and the production of the BS106 polypeptide. For triple helix, see, for example, Lee et al, Nuc. Acids Res. 6: 3073 (1979); Cooney et al, Science 241: 456(1988); and Dervanetal, Science 251: 1360(1991) The antisense RNA oligonucleotide hybridizes to the mRNA in vivo and blocks translation of a mRNA molecule into the BS106 polypeptide. For antisense, see, for example, Okano, J. Neurochem. 56: 560 (1991); and “Oligodeoxynucleotides as Antisense Inhibitors of Gene Expression,” CRC Press, Boca Raton, Fla. (1988). Antisense oligonucleotides act with greater efficacy when modified to contain artificial internucleotide linkages which render the molecule resistant to nucleolytic cleavage. Such artificial internucleotide linkages include, but are not limited to, methylphosphonate, phosphorothiolate and phosphoroamydate internucleotide linkages.

Recombinant Technology.

The present invention provides host cells and expression vectors comprising BS106 polynucleotides of the present invention and methods for the production of the polypeptide(s) they encode. Such methods comprise culturing the host cells under conditions suitable for the expression of the BS106 polynucleotide and recovering the BS106 polypeptide from the cell culture.

The present invention also provides vectors which include BS106 polynucleotides of the present invention, host cells which are genetically engineered with vectors of the present invention and the production of polypeptides of the present invention by recombinant techniques.

Host cells are genetically engineered (transfected, transduced or transformed) with the vectors of this invention which may be cloning vectors or expression vectors. The vector may be in the form of a plasmid, a viral particle, a phage, etc. The engineered host cells can be cultured in conventional nutrient media modified as appropriate for activating promoters, selecting transfected cells, or amplifying BS106 gene(s). The culture conditions, such as temperature, pH and the like, are those previously used with the host cell selected for expression, and will be apparent to the ordinarily skilled artisan.

The polynucleotides of the present invention may be employed for producing a polypeptide by recombinant techniques. Thus, the polynucleotide sequence may be included in any one of a variety of expression vehicles, in particular vectors or plasmids for expressing a polypeptide. Such vectors include chromosomal, nonchromosomal and synthetic DNA sequences, e.g., derivatives of SV40; bacterial plasmids; phage DNA; yeast plasmids; vectors derived from combinations of plasmids and phage DNA, viral DNA such as vaccinia, adenovirus, fowl pox virus and pseudorabies. However, any other plasmid or vector may be used so long as it is replicable and viable in the host.

The appropriate DNA sequence may be inserted into the vector by a variety of procedures. In general, the DNA sequence is inserted into appropriate restriction endonuclease sites by procedures known in the art. Such procedures and others are deemed to be within the scope of those skilled in the art. The DNA sequence in the expression vector is operatively linked to an appropriate expression control sequence(s) (promoter) to direct mRNA synthesis. Representative examples of such promoters include, but are not limited to, the LTR or the SV40 promoter, the E. coli lac or trp, the phage lambda P sub L promoter and other promoters known to control expression of genes in prokaryotic or eukaryotic cells or their viruses. The expression vector also contains a ribosome binding site for translation initiation and a transcription terminator. The vector may also include appropriate sequences for amplifying expression. In addition, the expression vectors preferably contain a gene to provide a phenotypic trait for selection of transfected host cells such as dihydrofolate reductase or neomycin resistance for eukaryotic cell culture, or such as tetracycline or ampicillin resistance in E. coli.

The vector containing the appropriate DNA sequence as hereinabove described, as well as an appropriate promoter or control sequence, may be employed to transfect an appropriate host to permit the host to express the protein. As representative examples of appropriate hosts, there may be mentioned: bacterial cells, such as E. coli, Salmonella typhimurium; Streptomyces sp.; fungal cells, such as yeast; insect cells such as Drosophila and Sf9; animal cells such as CHO, COS or Bowes melanoma; plant cells, etc. The selection of an appropriate host is deemed to be within the scope of those skilled in the art from the teachings provided herein.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art and are commercially available. The following vectors are provided by way of example. Bacterial: pINCY (Incyte Pharmaceuticals Inc., Palo Alto, Calif.), pSPORT1 (Life Technologies, Gaithersburg, Md.), pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK233-3, pDR540, pRIT5 (Pharmacia); Eukaryotic: pWLneo, pSV2cat, pOG44, pXT1, pSG (Stratagene) pSVK3, pBPV, pMSG, pSVL (Pharmacia). However, any other plasmid or vector may be used as long as it is replicable and viable in the host.

Plasmid pINCY is generally identical to the plasmid pSPORT1 (available from Life Technologies, Gaithersburg, Md.) with the exception that it has two modifications in the polylinker (multiple cloning site). These modifications are (1) it lacks a HindIII restriction site and (2) its EcoRI restriction site lies at a different location. pINCY is created from pSPORT1 by cleaving pSPORT1 with both HindIII and EcoRI and replacing the excised fragment of the polylinker with synthetic DNA fragments (SEQUENCE ID NO 6 and SEQUENCE ID NO 7). This replacement may be made in any manner known to those of ordinary skill in the art. For example, the two nucleotide sequences, SEQUENCE ID NO 8 and SEQUENCE ID NO 7, may be generated synthetically with 5′ terminal phosphates, mixed together, and then ligated under standard conditions for performing staggered end ligations into the pSPORT1 plasmid cut with HindIII and EcoRI. Suitable host cells (such as E. coli DH5∝ cells) then are transfected with the ligated DNA and recombinant clones are selected for ampicillin resistance. Plasmid DNA then is prepared from individual clones and subjected to restriction enzyme analysis or DNA sequencing in order to confirm the presence of insert sequences in the proper orientation. Other cloning strategies known to the ordinary artisan also may be employed.

Promoter regions can be selected from any desired gene using CAT (chloramphenicol transferase) vectors or other vectors with selectable markers. Two appropriate vectors are pKK232-8 and pCM7. Particular named bacterial promoters include lac, lacZ, T3, SP6, T7, gpt, lambda P sub R, P sub L and trp. Eukaryotic promoters include cytomegalovirus (CMV) immediate early, herpes simplex virus (HSV) thymidine kinase, early and late SV40, LTRs from retroviruses and mouse metallothionein-I. Selection of the appropriate vector and promoter is well within the level of ordinary skill in the art.

In a further embodiment, the present invention provides host cells containing the above-described construct. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (L. Davis et al., “Basic Methods in Molecular Biology,” 2nd edition, Appleton and Lang, Paramount Publishing, East Norwalk, Conn. (1994)).

The constructs in host cells can be used in a conventional manner to produce the gene product encoded by the recombinant sequence. Alternatively, the polypeptides of the invention can be synthetically produced by conventional peptide synthesizers.

Recombinant proteins can be expressed in mammalian cells, yeast, bacteria, or other cells, under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook et al., Molecular Cloning: A Laboratory Manual, Second Edition, (Cold Spring Harbor, N.Y., 1989), which is hereby incorporated by reference.

Transcription of a DNA encoding the polypeptide(s) of the present invention by higher eukaryotes is increased by inserting an enhancer sequence into the vector. Enhancers are cis-acting elements of DNA, usually about from 10 to 300 bp, that act on a promoter to increase its transcription. Examples include the SV40 enhancer on the late side of the replication origin (bp 100 to 270), a cytomegalovirus early promoter enhancer, a polyoma enhancer on the late side of the replication origin and adenovirus enhancers.

Generally, recombinant expression vectors will include origins of replication and selectable markers permitting transfection of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), alpha factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transfection include E. coli, Bacillus subtilis, Salmonella typhimurium and various species within the genera Pseudomonas, Streptomyces and Staphylococcus, although, others may also be employed as a routine matter of choice.

Useful expression vectors for bacterial use comprise a selectable marker and bacterial origin of replication derived from plasmids comprising genetic elements of the well-known cloning vector pBR322 (ATCC 37017). Other vectors include but are not limited to PKK223-3 (Pharmacia Fine Chemicals, Uppsala, Sweden) and GEM1 (Promega Biotec, Madison, Wis.). These pBR322 “backbone” sections are combined with an appropriate promoter and the structural sequence to be expressed.

Following transfection of a suitable host and growth of the host to an appropriate cell density, the selected promoter is derepressed by appropriate means (e.g., temperature shift or chemical induction), and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents; such methods are well-known to the ordinary artisan.

Various mammalian cell culture systems can also be employed to express recombinant protein. Examples of mammalian expression systems include the COS-7 lines of monkey kidney fibroblasts described by Gluzman, Cell 23: 175 (1981), and other cell lines capable of expressing a compatible vector, such as the C127, HEK-293, 3T3, CHO, HeLa and BHK cell lines. Mammalian expression vectors will comprise an origin of replication, a suitable promoter and enhancer and also any necessary ribosome binding sites, polyadenylation sites, splice donor and acceptor sites, transcriptional termination sequences and 5′ flanking nontranscribed sequences. DNA sequences derived from the SV40 viral genome, for example, SV40 origin, early promoter, enhancer, splice, and polyadenylation sites may be used to provide the required nontranscribed genetic elements. Representative, useful vectors include pRc/CMV and pcDNA3 (available from Invitrogen, San Diego, Calif.).

BS106 polypeptides are recovered and purified from recombinant cell cultures by known methods including affinity chromatography, ammonium sulfate or ethanol precipitation, acid extraction, anion or cation exchange chromatography, phosphocellulose chromatography, hydrophobic interaction chromatography, hydroxyapatite chromatography or lectin chromatography. It is preferred to have low concentrations (approximately 0.1-5 mM) of calcium ion present during purification (Price, et al., J. Biol. Chem. 244: 917 (1969)). Protein refolding steps can be used, as necessary, in completing configuration of the polypeptide. Finally, high performance liquid chromatography (HPLC) can be employed for final purification steps.

Thus, polypeptides of the present invention may be naturally purified products expressed from a high expressing cell line, or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated with mammalian or other eukaryotic carbohydrates or may be non-glycosylated. The polypeptides of the invention may also include an initial methionine amino acid residue.

The starting plasmids can be constructed from available plasmids in accord with published, known procedures. In addition, equivalent plasmids to those described are known in the art and will be apparent to the ordinarily skilled artisan.

The following is the general procedure for the isolation and analysis of cDNA clones. In a particular embodiment disclosed herein, mRNA was isolated from breast tissue and used to generate the cDNA library. Breast tissue was obtained from patients by surgical resection and was classified as tumor or non-tumor tissue by a pathologist.

The cDNA inserts from random isolates of the breast tissue libraries were sequenced in part, analyzed in detail as set forth in the Examples and are disclosed in the Sequence Listing as SEQUENCE ID NO 1, SEQUENCE ID NO 2, SEQUENCE ID NO 3, and SEQUENCE ID NO 4. The consensus sequence of these inserts is presented as SEQUENCE ID NO 6. The full-length sequence is SEQUINCE ID NO 5. These polynucleotides may contain an entire open reading frame with or without associated regulatory sequences for a particular gene, or they may encode only a portion of the gene of interest. This is attributed to the fact that many genes are several hundred and sometimes several thousand bases in length and, with current technology, cannot be cloned in their entirety because of vector limitations, incomplete reverse transcription of the first strand, or incomplete replication of the second strand. Contiguous, secondary clones containing additional nucleotide sequences may be obtained using a variety of methods known to those of skill in the art.

Methods for DNA sequencing are well-known in the art. Conventional enzymatic methods employ DNA polymerase, Klenow fragment, Sequenase (US Biochemical Corp, Cleveland, Ohio) or Taq polymerase to extend DNA chains from an oligonucleotide primer annealed to the DNA template of interest. Methods have been developed for the use of both single-stranded and double-stranded templates. The chain termination reaction products may be electrophoresed on urea/polyacrylamide gels and detected either by autoradiography (for radionucleotide labeled precursors) or by fluorescence (for fluorescent-labeled precursors). Recent improvements in mechanized reaction preparation, sequencing and analysis using the fluorescent detection method have permitted expansion in the number of sequences that can be determined per day using machines such as the Applied Biosystems 377 DNA Sequencers (Applied Biosystems, Foster City, Calif.).

The reading frame of the nucleotide sequence can be ascertained by several types of analyses. First, reading frames contained within the coding sequence can be analyzed for the presence of start codon ATG and stop codons TGA, TAA or TAG. Typically, one reading frame will continue throughout the major portion of a cDNA sequence while other reading frames tend to contain numerous stop codons. In such cases, reading frame determination is straightforward. In other more difficult cases, further analysis is required.

Algorithms have been created to analyze the occurrence of individual nucleotide bases at each putative codon triplet. See, for example J. W. Fickett, Nuc Acids Res 10: 5303 (1982). Coding DNA for particular organisms (bacteria, plants and animals) tends to contain certain nucleotides within certain triplet periodicities, such as a significant preference for pyrimidines in the third codon position. These preferences have been incorporated into widely available software which can be used to determine coding potential (and frame) of a given stretch of DNA. The algorithm-derived information combined with start/stop codon information can be used to determine proper frame with a high degree of certainty. This, in turn, readily permits cloning of the sequence in the correct reading frame into appropriate expression vectors.

The nucleic acid sequences disclosed herein may be joined to a variety of other polynucleotide sequences and vectors of interest by means of well-established recombinant DNA techniques. See J. Sambrook et al., supra. Vectors of interest include cloning vectors, such as plasmids, cosmids, phage derivatives, phagemids, as well as sequencing, replication and expression vectors, and the like. In general, such vectors contain an origin of replication functional in at least one organism, convenient restriction endonuclease digestion sites and selectable markers appropriate for particular host cells. The vectors can be transferred by a variety of means known to those of skill in the art into suitable host cells which then produce the desired DNA, RNA or polypeptides.

Occasionally, sequencing or random reverse transcription errors will mask the presence of the appropriate open reading frame or regulatory element. In such cases, it is possible to determine the correct reading frame by attempting to express the polypeptide and determining the amino acid sequence by standard peptide mapping and sequencing techniques. See, F. M. Ausubel et al., Current Protocols in Molecular Biology, John Wiley & Sons, New York, N.Y. (1989). Additionally, the actual reading frame of a given nucleotide sequence may be determined by transfection of host cells with vectors containing all three potential reading frames. Only those cells with the nucleotide sequence in the correct reading frame will produce a peptide of the predicted length.

The nucleotide sequences provided herein have been prepared by current, state-of-the-art, automated methods and as such may contain unidentified nucleotides. These will not present a problem to those skilled in the art who wish to practice the invention. Several methods employing standard recombinant techniques, described in J. Sambrook (supra) or periodic updates thereof, may be used to complete the missing sequence information. The same techniques used for obtaining a full length sequence, as described herein, may be used to obtain nucleotide sequences.

Expression of a particular cDNA may be accomplished by subcloning the cDNA into an appropriate expression vector and transfecting this vector into an appropriate expression host. The cloning vector used for the generation of the breast tissue cDNA library can be used for transcribing mRNA of a particular cDNA and contains a promoter for beta-galactosidase, an amino-terminal met and the subsequent seven amino acid residues of beta-galactosidase. Immediately following these eight residues is an engineered bacteriophage promoter, useful for artificial priming and transcription, as well as a number of unique restriction sites, including EcoRI, for cloning. The vector can be transfected into an appropriate host strain of E. coli.

Induction of the isolated bacterial strain with isopropylthiogalactoside (IPTG) using standard methods will produce a fusion protein which contains the first seven residues of beta-galactosidase, about 15 residues of linker and the peptide encoded within the cDNA. Since cDNA clone inserts are generated by an essentially random process, there is one chance in three that the included cDNA will lie in the correct frame for proper translation. If the cDNA is not in the proper reading frame, the correct frame can be obtained by deletion or insertion of an appropriate number of bases by well known methods including in vitro mutagenesis, digestion with exonuclease III or mung bean nuclease, or oligonucleotide linker inclusion.

The cDNA can be shuttled into other vectors known to be useful for expression of protein in specific hosts. Oligonucleotide primers, containing cloning sites and segments of DNA sufficient to hybridize to stretches at both ends of the target cDNA, can be synthesized chemically by standard methods. These primers can then be used to amplify the desired gene segments by PCR. The resulting new gene segments can be digested with appropriate restriction enzymes under standard conditions and isolated by gel electrophoresis. Alternately, similar gene segments can be produced by digestion of the cDNA with appropriate restriction enzymes and filling in the missing gene segments with chemically synthesized oligonucleotides. Segments of the coding sequence from more than one gene can be ligated together and cloned in appropriate vectors to optimize expression of recombinant sequence.

Suitable expression hosts for such chimeric molecules include but are not limited to, mammalian cells such as Chinese Hamster Ovary (CHO) and human embryonic kidney (HEK) 293 cells, insect cells such as Sf9 cells, yeast cells such as Saccharomyces cerevisiae and bacteria such as E. coli. For each of these cell systems, a useful expression vector may also include an origin of replication to allow propagation in bacteria and a selectable marker such as the beta-lactamase antibiotic resistance gene to allow selection in bacteria. In addition, the vectors may include a second selectable marker, such as the neomycin phosphotransferase gene, to allow selection in transfected eukaryotic host cells. Vectors for use in eukaryotic expression hosts may require the addition of 3′ poly A tail if the sequence of interest lacks poly A.

Additionally, the vector may contain promoters or enhancers which increase gene expression. Such promoters are host specific and include, but are not limited to, MMTV, SV40, or metallothionine promoters for CHO cells; trp, lac, tac or T7 promoters for bacterial hosts; or alpha factor, alcohol oxidase or PGH promoters for yeast. Adenoviral vectors with or without transcription enhancers, such as the rous sarcoma virus (RSV) enhancer, may be used to drive protein expression in mammalian cell lines. Once homogeneous cultures of recombinant cells are obtained, large quantities of recombinantly produced protein can be recovered from the conditioned medium and analyzed using chromatographic methods well known in the art. An alternative method for the production of large amounts of secreted protein involves the transfection of mammalian embryos and the recovery of the recombinant protein from milk produced by transgenic cows, goats, sheep, etc. Polypeptides and closely related molecules may be expressed recombinantly in such a way as to facilitate protein purification. One approach involves expression of a chimeric protein which includes one or more additional polypeptide domains not naturally present on human polypeptides. Such purification-facilitating domains include, but are not limited to, metal-chelating peptides such as histidine-tryptophan domains that allow purification on immobilized metals, protein A domains that allow purification on immobilized immunoglobulin and the domain utilized in the FLAGS extension/affinity purification system (Immunex Corp, Seattle, Wash.). The inclusion of a cleavable linker sequence such as Factor XA or enterokinase from Invitrogen (San Diego, Calif.) between the polypeptide sequence and the purification domain may be useful for recovering the polypeptide.

Immunoassays

BS106 polypeptides, including fragments, derivatives, and analogs thereof, or cells expressing such polypeptides, can be utilized in a variety of assays, many of which are described herein, for the detection of antibodies to breast tissue. They also can be used as immunogens to produce antibodies. These antibodies can be, for example, polyclonal or monoclonal antibodies, chimeric, single chain and humanized antibodies, as well as Fab fragments, or the product of an Fab expression library. Various procedures known in the art may be used for the production of such antibodies and fragments.

For example, antibodies generated against a polypeptide comprising a sequence of the present invention can be obtained by direct injection of the polypeptide into an animal or by administering the polypeptide to an animal such as a mouse, rabbit, goat or human. A mouse, rabbit or goat is preferred. The polypeptide is selected from the group consisting of SEQUENCE ID NOS 20-33, and fragments thereof. The antibody so obtained then will bind the polypeptide itself. In this manner, even a sequence encoding only a fragment of the polypeptide can be used to generate antibodies that bind the native polypeptide. Such antibodies then can be used to isolate the polypeptide from test samples such as tissue suspected of containing that polypeptide. For preparation of monoclonal antibodies, any technique which provides antibodies produced by continuous cell line cultures can be used. Examples include the hybridoma technique as described by Kohler and Milstein, Nature 256: 495-497 (1975), the trioma technique, the human B-cell hybridoma technique as described by Kozbor et al, Immun. Today 4: 72 (1983) and the EBV-hybridoma technique to produce human monoclonal antibodies as described by Cole et al., in Monoclonal Antibodies and Cancer Therapy, Alan R. Liss, Inc, New York, N.Y., pp. 77-96 (1985). Techniques described for the production of single chain antibodies can be adapted to produce single chain antibodies to immunogenic polypeptide products of this invention. See, for example, U.S. Pat. No. 4,946,778, which is incorporated herein by reference.

Various assay formats may utilize the antibodies of the present invention, including “sandwich” immunoassays and probe assays. For example, the antibodies of the present invention, or fragments thereof, can be employed in various assay systems to determine the presence, if any, of BS106 antigen in a test sample. For example, in a first assay format, a polyclonal or monoclonal antibody or fragment thereof, or a combination of these antibodies, which has been coated on a solid phase, is contacted with a test sample, to form a first mixture. This first mixture is incubated for a time and under conditions sufficient to form antigen/antibody complexes. Then, an indicator reagent comprising a monoclonal or a polyclonal antibody or a fragment thereof, or a combination of these antibodies, to which a signal generating compound has been attached, is contacted with the antigen/antibody complexes to form a second mixture. This second mixture then is incubated for a time and under conditions sufficient to form antibody/antigen/antibody complexes. The presence of BS106 antigen in the test sample and captured on the solid phase, if any, is determined by detecting the measurable signal generated by the signal generating compound. The amount of BS106 antigen present in the test sample is proportional to the signal generated.

In an alternative assay format, a mixture is formed by contacting: (1) a polyclonal antibody, monoclonal antibody, or fragment thereof, which specifically binds to BS106 antigen, or a combination of such antibodies bound to a solid support; (2) the test sample; and (3) an indicator reagent comprising a monoclonal antibody, polyclonal antibody, or fragment thereof, which specifically binds to a different BS106 antigen (or a combination of these antibodies) to which a signal generating compound is attached. This mixture is incubated for a time and under conditions sufficient to form antibody/antigen/antibody complexes. The presence, if any, of BS106 antigen present in the test sample and captured on the solid phase is determined by detecting the measurable signal generated by the signal generating compound. The amount of BS106 antigen present in the test sample is proportional to the signal generated.

In another assay format, one or a combination of at least two monoclonal antibodies of the invention can be employed as a competitive probe for the detection of antibodies to BS106 antigen. For example, BS106 polypeptides such as the recombinant antigens disclosed herein, either alone or in combination, are coated on a solid phase. A test sample suspected of containing antibody to BS106 antigen then is incubated with an indicator reagent comprising a signal generating compound and at least one monoclonal antibody of the invention for a time and under conditions sufficient to form antigen/antibody complexes of either the test sample and indicator reagent bound to the solid phase or the indicator reagent bound to the solid phase. The reduction in binding of the monoclonal antibody to the solid phase can be quantitatively measured.

In yet another detection method, each of the monoclonal or polyclonal antibodies of the present invention can be employed in the detection of BS106 antigens in tissue sections, as well as in cells, by immunohistochemical analysis. Cytochemical analysis wherein these antibodies are labeled directly (with, for example, fluorescein, colloidal gold, horseradish peroxidase, alkaline phosphatase, etc.) or are labeled by using secondary labeled anti-species antibodies (with various labels as exemplified herein) to track the histopathology of disease also are within the scope of the present invention.

In addition, these monoclonal antibodies can be bound to matrices similar to CNBr-activated Sepharose and used for the affinity purification of specific BS106 polypeptides from cell cultures or biological tissues such as to purify recombinant and native BS106 proteins.

The monoclonal antibodies of the invention also can be used for the generation of chimeric antibodies for therapeutic use, or other similar applications.

The monoclonal antibodies or fragments thereof can be provided individually to detect BS106 antigens. Combinations of the monoclonal antibodies (and fragments thereof) provided herein also may be used together as components in a mixture or “cocktail” of at least one BS106 antibody of the invention, along with antibodies which specifically bind to other BS106 regions, each antibody having different binding specificities. Thus, this cocktail can include the monoclonal antibodies of the invention which are directed to BS106 polypeptides disclosed herein and other monoclonal antibodies specific to other antigenic determinants of BS106 antigens or other related proteins.

The polyclonal antibody or fragment thereof which can be used in the assay formats should specifically bind to a BS106 polypeptide or other BS106 polypeptides additionally used in the assay. The polyclonal antibody used preferably is of mammalian origin such as, human, goat, rabbit or sheep polyclonal antibody which binds BS106 polypeptide. Most preferably, the polyclonal antibody is of rabbit origin. The polyclonal antibodies used in the assays can be used either alone or as a cocktail of polyclonal antibodies. Since the cocktails used in the assay formats are comprised of either monoclonal antibodies or polyclonal antibodies having different binding specificity to BS106 polypeptides, they are useful for the detecting, diagnosing, staging, monitoring, prognosticating, preventing or treating, or determining the predisposition to, diseases and conditions of the breast such as breast cancer.

It is contemplated and within the scope of the present invention that BS106 antigen may be detectable in assays by use of a recombinant antigen as well as by use of a synthetic peptide or purified peptide, which peptide comprises an amino acid sequence of BS106. The amino acid sequence of such a polypeptide is selected from the group consisting of SEQUENCE ID NOS 20-33, and fragments thereof. It also is within the scope of the present invention that different synthetic, recombinant or purified peptides, identifying different epitopes of BS106, can be used in combination in an assay for the detecting, diagnosing, staging, monitoring, prognosticating, preventing or treating, or determining the predisposition to diseases and conditions of the breast such as breast cancer. In this case, all of these peptides can be coated onto one solid phase; or each separate peptide may be coated onto separate solid phases, such as microparticles, and then combined to form a mixture of peptides which can be later used in assays. Furthermore, it is contemplated that multiple peptides which define epitopes from different antigens may be used for the detection, diagnosis, staging, monitoring, prognosis, prevention or treatment of, or determining the predisposition to, diseases and conditions of the breast, such as breast cancer. Peptides coated on solid phases or labeled with detectable labels are then allowed to compete with those present in a patient sample (if any) for a limited amount of antibody. A reduction in binding of the synthetic, recombinant, or purified peptides to the antibody (or antibodies) is an indication of the presence of BS106 antigen in the patient sample. The presence of BS106 antigen indicates the presence of breast tissue disease, especially breast cancer, in the patient. Variations of assay formats are known to those of ordinary skill in the art and many are discussed herein below.

In another assay format, the presence of anti-BS106 antibody and/or BS106 antigen can be detected in a simultaneous assay, as follows. A test sample is simultaneously contacted with a capture reagent of a first analyte, wherein said capture reagent comprises a first binding member specific for a first analyte attached to a solid phase and a capture reagent for a second analyte, wherein said capture reagent comprises a first binding member for a second analyte attached to a second solid phase, to thereby form a mixture. This mixture is incubated for a time and under conditions sufficient to form capture reagent/first analyte and capture reagent/second analyte complexes. These so-formed complexes then are contacted with an indicator reagent comprising a member of a binding pair specific for the first analyte labeled with a signal generating compound and an indicator reagent comprising a member of a binding pair specific for the second analyte labeled with a signal generating compound to form a second mixture. This second mixture is incubated for a time and under conditions sufficient to form capture reagent/first analyte/indicator reagent complexes and capture reagent/second analyte/indicator reagent complexes. The presence of one or more analytes is determined by detecting a signal generated in connection with the complexes formed on either or both solid phases as an indication of the presence of one or more analytes in the test sample. In this assay format, recombinant antigens derived from the expression systems disclosed herein may be utilized, as well as monoclonal antibodies produced from the proteins derived from the expression systems as disclosed herein. For example, in this assay system, BS106 antigen can be the first analyte. Such assay systems are described in greater detail in EP Publication No. 0473065.

In yet other assay formats, the polypeptides disclosed herein may be utilized to detect the presence of antibody against BS106 antigen in test samples. For example, a test sample is incubated with a solid phase to which at least one polypeptide such as a recombinant protein or synthetic peptide has been attached. The polypeptide is selected from the group consisting of SEQUENCE ID NOS 20-33, and fragments thereof. These are reacted for a time and under conditions sufficient to form antigen/antibody complexes. Following incubation, the antigen/antibody complex is detected. Indicator reagents may be used to facilitate detection, depending upon the assay system chosen. In another assay format, a test sample is contacted with a solid phase to which a recombinant protein produced as described herein is attached, and also is contacted with a monoclonal or polyclonal antibody specific for the protein, which preferably has been labeled with an indicator reagent. After incubation for a time and under conditions sufficient for antibody/antigen complexes to form, the solid phase is separated from the free phase, and the label is detected in either the solid or free phase as an indication of the presence of antibody against BS106 antigen. Other assay formats utilizing the recombinant antigens disclosed herein are contemplated. These include contacting a test sample with a solid phase to which at least one antigen from a first source has been attached, incubating the solid phase and test sample for a time and under conditions sufficient to form antigen/antibody complexes, and then contacting the solid phase with a labeled antigen, which antigen is derived from a second source different from the first source. For example, a recombinant protein derived from a first source such as E. coli is used as a capture antigen on a solid phase, a test sample is added to the so-prepared solid phase, and following standard incubation and washing steps as deemed or required, a recombinant protein derived from a different source (i.e., non-E. coli) is utilized as a part of an indicator reagent which subsequently is detected. Likewise, combinations of a recombinant antigen on a solid phase and synthetic peptide in the indicator phase also are possible. Any assay format which utilizes an antigen specific for BS106 produced or derived from a first source as the capture antigen and an antigen specific for BS106 from a different second source is contemplated. Thus, various combinations of recombinant antigens, as well as the use of synthetic peptides, purified proteins and the like, are within the scope of this invention. Assays such as this and others are described in U.S. Pat. No. 5,254,458, which enjoys common ownership and is incorporated herein by reference.

Other embodiments which utilize various other solid phases also are contemplated and are within the scope of this invention. For example, ion capture procedures for immobilizing an immobilizable reaction complex with a negatively charged polymer (described in EP publication 0326100 and EP publication No. 0406473), can be employed according to the present invention to effect a fast solution-phase immunochemical reaction. An immobilizable immune complex is separated from the rest of the reaction mixture by ionic interactions between the negatively charged poly-anion/immune complex and the previously treated, positively charged porous matrix and detected by using various signal generating systems previously described, including those described in chemiluminescent signal measurements as described in EPO Publication No. 0 273,115.

Also, the methods of the present invention can be adapted for use in systems which utilize microparticle technology including automated and semi-automated systems wherein the solid phase comprises a microparticle (magnetic or non-magnetic). Such systems include those described in, for example, published EPO applications Nos. EP 0 425 633 and EP 0 424 634, respectively.

The use of scanning probe microscopy (SPM) for immunoassays also is a technology to which the monoclonal antibodies of the present invention are easily adaptable. In scanning probe microscopy, particularly in atomic force microscopy, the capture phase, for example, at least one of the monoclonal antibodies of the invention, is adhered to a solid phase and a scanning probe microscope is utilized to detect antigen/antibody complexes which may be present on the surface of the solid phase. The use of scanning tunneling microscopy eliminates the need for labels which normally must be utilized in many immunoassay systems to detect antigen/antibody complexes. The use of SPM to monitor specific binding reactions can occur in many ways. In one embodiment, one member of a specific binding partner (analyte specific substance which is the monoclonal antibody of the invention) is attached to a surface suitable for scanning. The attachment of the analyte specific substance may be by adsorption to a test piece which comprises a solid phase of a plastic or metal surface, following methods known to those of ordinary skill in the art. Or, covalent attachment of a specific binding partner (analyte specific substance) to a test piece which test piece comprises a solid phase of derivatized plastic, metal, silicon, or glass may be utilized. Covalent attachment methods are known to those skilled in the art and include a variety of means to irreversibly link specific binding partners to the test piece. If the test piece is silicon or glass, the surface must be activated prior to attaching the specific binding partner. Also, polyelectrolyte interactions may be used to immobilize a specific binding partner on a surface of a test piece by using techniques and chemistries. The preferred method of attachment is by covalent means. Following attachment of a specific binding member, the surface may be further treated with materials such as serum, proteins, or other blocking agents to minimize non-specific binding. The surface also may be scanned either at the site of manufacture or point of use to verify its suitability for assay purposes. The scanning process is not anticipated to alter the specific binding properties of the test piece.

While the present invention discloses the preference for the use of solid phases, it is contemplated that the reagents such as antibodies, proteins and peptides of the present invention can be utilized in non-solid phase assay systems. These assay systems are known to those skilled in the art, and are considered to be within the scope of the present invention.

It is contemplated that the reagent employed for the assay can be provided in the form of a test kit with one or more containers such as vials or bottles, with each container containing a separate reagent such as a probe, primer, monoclonal antibody or a cocktail of monoclonal antibodies, or a polypeptide (e.g. recombinantly, synthetically produced or purified) employed in the assay. The polypeptide is selected from the group consisting of SEQUENCE ID NOS 20-33, and fragments thereof. Other components such as buffers, controls and the like, known to those of ordinary skill in art, may be included in such test kits. It also is contemplated to provide test kits which have means for collecting test samples comprising accessible body fluids, e.g., blood, urine, saliva and stool. Such tools useful for collection (“collection materials”) include lancets and absorbent paper or cloth for collecting and stabilizing blood; swabs for collecting and stabilizing saliva; cups for collecting and stabilizing urine or stool samples. Collection materials, papers, cloths, swabs, cups and the like, may optionally be treated to avoid denaturation or irreversible adsorption of the sample. The collection materials also may be treated with or contain preservatives, stabilizers or antimicrobial agents to help maintain the integrity of the specimens. Test kits designed for the collection, stabilization and preservation of test specimens obtained by surgery or needle biopsy are also useful. It is contemplated that all kits may be configured in two components which can be provided separately; one component for collection and transport of the specimen and the other component for the analysis of the specimen. The collection component, for example, can be provided to the open market user while the components for analysis can be provided to others such as laboratory personnel for determination of the presence, absence or amount of analyte. Further, kits for the collection, stabilization and preservation of test specimens may be configured for use by untrained personnel and may be available in the open market for use at home with subsequent transportation to a laboratory for analysis of the test sample.

In Vivo Antibody Use

Antibodies of the present invention can be used in vivo; that is, they can be injected into patients suspected of having or having diseases of the breast for diagnostic or therapeutic uses. The use of antibodies for in vivo diagnosis is well known in the art. Sumerdon et al., Nucl. Med. Biol 17: 247-254 (1990) have described an optimized antibody-chelator for the radioimmunoscintographic imaging of carcinoembryonic antigen (CEA) expressing tumors using Indium-111 as the label. Griffin et al., J Clin One 9: 631-640 (1991) have described the use of this agent in detecting tumors in patients suspected of having recurrent colorectal cancer. The use of similar agents with paramagnetic ions as labels for magnetic resonance imaging is know in the art (R. B. Lauffer, Magnetic Resonance in Medicine 22: 339-342 (1991). It is anticipated that antibodies directed against BS106 antigen can be injected into patients suspected of having a disease of the breast such as breast cancer for the purpose of diagnosing or staging the disease status of the patient. The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used. Localization of the label within the breast or external to the breast may allow determination of spread of the disease. The amount of label within the breast may allow determination of the presence or absence of cancer of the breast.

For patients known to have a disease of the breast, injection of an antibody directed against BS106 antigen may have therapeutic benefit. The antibody may exert its effect without the use of attached agents by binding to BS106 antigen expressed on or in the tissue or organ. Alternatively, the antibody may be conjugated to cytotoxic agents such as drugs, toxins, or radionuclides to enhance its therapeutic effect. Garnett and Baldwin, Cancer Research 46: 2407-2412 (1986) have described the preparation of a drug-monoclonal antibody conjugate. Pastan et al., Cell 47: 641-648 (1986) have reviewed the use of toxins conjugated to monoclonal antibodies for the therapy of various cancers. Goodwin and Meares, Cancer Supplement 80: 2675-2680 (1997) have described the use of Yittrium-90 labeled monoclonal antibodies in various strategies to maximize the dose to tumor while limiting normal tissue toxicity. Other known cytotoxic radionuclides include Copper-67, Iodine-131, and Rhenium-186 all of which can be used to label monoclonal antibodies directed against BS106 antigen for the treatment of cancer of the breast.

E. coli bacteria (clone 1662885) was deposited on Nov. 20, 1996 with the American Type Culture Collection (A.T.C.C.), 10801 University Blvd., Manassas, Va. The deposit was made under the terms of the Budapest Treaty and will be maintained for a period of thirty (30) years from the date of deposit, or for five (5) years after the last request for the deposit, or for the enforceable period of the U.S. patent, whichever is longer. The deposit and any other deposited material described herein are provided for convenience only, and are not required to practice the present invention in view of the teachings provided herein. The cDNA sequence in all of the deposited material is incorporated herein by reference. Clone 1662885 was accorded A.T.C.C. Deposit No. 98256.

The present invention will now be described by way of examples, which are meant to illustrate, but not to limit, the scope of the present invention.

### EXAMPLES

### Example 1

### Identification of Breast Tissue Library BS106 Gene-Specific Clones

A. Library Comparison of Expressed Sequence Tags (EST's) or Transcript Images. Partial sequences of cDNA clone inserts, so-called “expressed sequence tags” (EST's), were derived from cDNA libraries made from breast tumor tissues, breast non-tumor tissues and numerous other tissues, both tumor and non-tumor and entered into a database (LIFESEQ™ database, available from Incyte Pharmaceuticals, Palo Alto, Calif.) as gene transcript images. See International Publication No. WO 95/20681. (A transcript image is a listing of the number of EST's for each of the represented genes in a given tissue library. EST's sharing regions of mutual sequence overlap are classified into clusters. A cluster is assigned a clone number from a representative 5′ EST. Often, a cluster of interest can be extended by comparing its consensus sequence with sequences of other EST's that did not meet the criteria for automated clustering. The alignment of all available clusters and single EST's represents a contig from which a consensus sequence is derived.) The transcript images then were evaluated to identify EST sequences that were representative primarily of the breast tissue libraries. These target clones then were ranked according to their abundance (occurrence) in the target libraries and their absence from background libraries. Higher abundance clones with low background occurrence were given higher study priority. EST's corresponding to the consensus sequence of BS106 were found in 50.8% (30 of 59) of breast tissue libraries. EST's corresponding to the consensus sequence, SEQUENCE ID NO 6 (or fragments thereof), were found in only 0.509% (5 of 983) of the other non-breast tissue libraries of the database. Therefore, the consensus sequence or fragment thereof was found more than 100 times more often in breast tissue than non-breast tissues. Overlapping clones 1662885 (SEQUENCE ID NO 1), 893988 (SEQUENCE ID NO 2), 901429 (SEQUENCE ID NO 3), 1209814 (SEQUENCE ID NO 4), respectively, were identified for further study. These represented the minimum number of clones that (along with the full-length sequence of clone 1662885 [designated as 1662885inh (SEQUENCE ID NO 5)] were needed to form the contig and from which the consensus sequence provided herein (SEQUENCE ID NO 6) was derived.

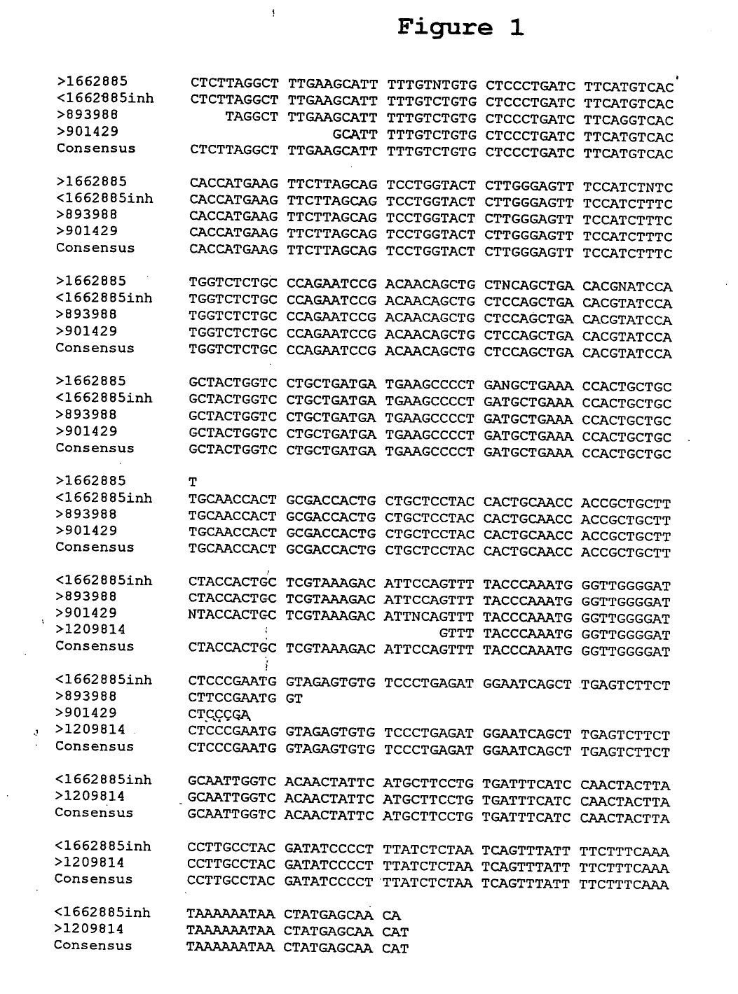

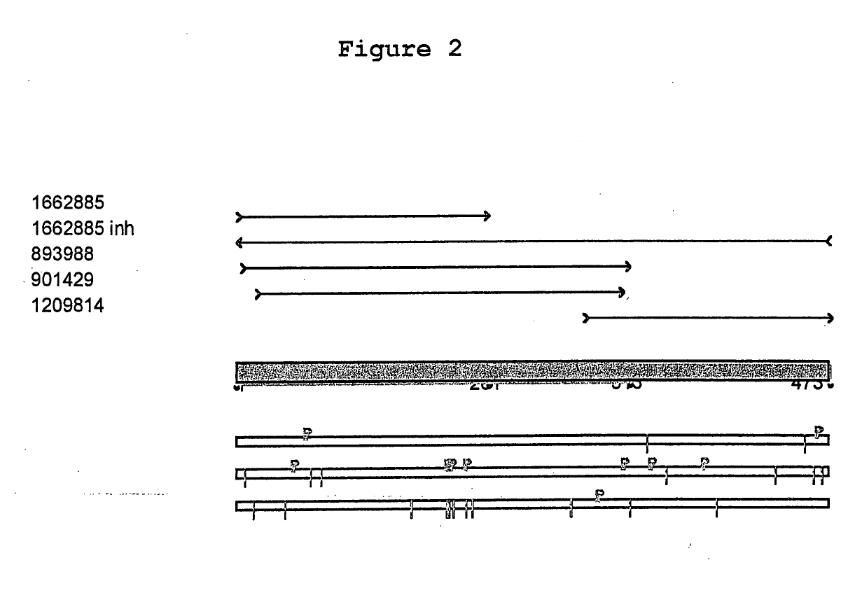

B. Generation of a Consensus Sequence. The nucleotide sequences of clones 1662885 (SEQUENCE ID NO 1), 893988 (SEQUENCE ID NO 2), 901429 (SEQUENCE ID NO 3), 1209814 (SEQUENCE ID NO 4), and the full-length sequence of clone 1662885 [designated as 1662885inh (SEQUENCE ID NO 5)] were entered in the Sequencher™ Program (available from Gene Codes Corporation, Ann Arbor, Mich.) in order to generate a nucleotide alignment (contig map) and then generate their consensus sequence (SEQUENCE ID NO 6). FIG. 1 shows the nucleotide sequence alignment of these clones and their resultant nucleotide consensus sequence (SEQUENCE ID NO 6). FIG. 2 presents the contig map depicting the clones 1662885 (SEQUENCE ID NO 1), 893988 (SEQUENCE ID NO 2), 901429 (SEQUENCE ID NO 3), 1209814 (SEQUENCE ID NO 4), and the full-length sequence of clone 1662885 [designated as 1662885inh (SEQUENCE ID NO 5)] which form overlapping regions of the BS106 gene and the resultant consensus nucleotide sequence (SEQUENCE ID NO 6) of these clones in a graphic display. Following this, a three-frame translation was performed on the consensus sequence (SEQUENCE ID NO 6). The first forward frame was found to have an open reading frame encoding a 90 residue amino acid sequence which is presented as SEQUENCE ID NO 20. The open reading frame corresponds to nucleotides 55-324 of SEQUENCE ID NO 6.

Analysis of the LIFESEQ™ database indicates a possible T/G polymorphism at position 45 in the consensus nucleotide sequence (SEQUENCE ID NO 6).

### Example 2

### Sequencing of BS106 EST-Specific Clones

The DNA sequence of clone 1662885inh of the BS106 gene contig was determined (SEQUENCE ID NO 5) using dideoxy termination sequencing with dye terminators following known methods [F. Sanger et al., PNAS U.S.A. 74: 5463 (1977)].

Because vectors such as pSPORT1 (Life Technologies, Gaithersburg, Md.) and pINCY (available from Incyte Pharmaceuticals, Inc., Palo Alto, Calif.) contain universal priming sites just adjacent to the 3′ and 5′ ligation junctions of the inserts, the inserts were sequenced in both directions using universal primers, SEQUENCE ID NO 9 and SEQUENCE ID NO 10 (New England Biolabs, Beverly, Mass. and Applied Biosystems Inc, Foster City, Calif., respectively). The sequencing reactions were run on a polyacrylamide denaturing gel, and the sequences were determined by an Applied Biosystems 377 Sequencer (available from Applied Biosystems, Foster City, Calif.). Additional sequencing primers, SEQUENCE ID NO 11 and SEQUENCE ID NO 12 were designed from sequence information of the consensus sequence, SEQUENCE ID NO 6. These primers then were used to determine the remaining DNA sequence of the cloned insert from each DNA strand, as previously described.

### Example 3

### Nucleic Acid

A. RNA Extraction from Tissue. Total RNA was isolated from breast tissues and from non-breast tissues. Various methods were utilized, including but not limited to the lithium chloride/urea technique, known in the art and described by Kato et al. (J. Virol. 61: 2182-2191, 1987), and TRIzol™ (Gibco-BRL, Grand Island, N.Y.).

Briefly, tissue was placed in a sterile conical tube on ice and 10-15 volumes of 3 M LiCl, 6 M urea, 5 mM EDTA, 0.1 M 2-mercaptoethanol and 50 mM Tris-HCl (pH 7.5) were added. The tissue was homogenized with a Polytron® homogenizer (Brinkman Instruments, Inc., Westbury, N.Y.) for 30-50 sec on ice. The solution was transferred to a 15 ml plastic centrifuge tube and placed overnight at −20° C. The tube was centrifuged for 90 min at 9,000×g at 0-4° C. and the supernatant was immediately decanted. Ten ml of 3 M LiCl were added and the tube was vortexed for 5 sec. The tube was centrifuged for 45 min at 1,000×g at 0-4° C. The decanting, resuspension in LiCl, and centrifugation were repeated and the final pellet was air dried and suspended in 2 ml of 1 mM EDTA, 0.5% SDS, 10 mM Tris (pH 7.5). Twenty microliters (20 μl) of Proteinase K (20 mg/ml) were added, and the solution was incubated for 30 min at 37° C. with occasional mixing. One-tenth volume (0.22-0.25 ml) of 3 M NaCl was added and the solution was vortexed before transfer into another tube containing 2 ml of phenol/chloroform/isoamyl alcohol (PCI). The tube was vortexed for 1-3 sec and centrifuged for 20 min at 3,000×g at 10° C. The PCI extraction was repeated and followed by two similar extractions with chloroform/isoamyl alcohol (CI). The final aqueous solution was transferred to a prechilled 15 ml Corex glass tube containing 6 ml of absolute ethanol. The tube was covered with parafilm and placed at −20° C. overnight. The tube was centrifuged for 30 min at 10,000×g at 0-4° C. and the ethanol supernatant was decanted immediately. The RNA pellet was washed four times with 10 ml of 75% ice-cold ethanol and the final pellet was air dried for 15 min at room temperature. The RNA was suspended in 0.5 ml of 10 mM TE (10 mM Tris-HCl, pH 7.6, 1 mM EDTA) and its concentration was determined spectrophotometrically. RNA samples were aliquoted and stored at −70° C. as ethanol precipitates.

The quality of the RNA was determined by agarose gel electrophoresis (see Example 5) and staining with 0.5 μg/ml ethidium bromide for one hour. RNA samples that did not contain intact ribosomal RNAs were excluded from the study.

Alternatively, for RT-PCR analysis, 1 ml of Ultraspec RNA reagent was added to 120 mg of pulverized tissue in a 2.0 ml polypropylene microfuge tube, homogenized with a Polytron® homogenizer (Brinkman Instruments, Inc., Westbury, N.Y.) for 50 sec and placed on ice for 5 min. Then, 0.2 ml of chloroform was added to each sample, followed by vortexing for 15 sec. The sample was placed on ice for another 5 min, followed by centrifugation at 12,000×g for 15 min at 4° C. The upper layer was collected and transferred to another RNase-free 2.0 ml microfuge tube. An equal volume of isopropanol was added to each sample, and the solution was placed on ice for 10 min. The sample was centrifuged at 12,000×g for 10 min at 4° C., and the supernatant was discarded. The remaining pellet was washed twice with cold 75% ethanol, resuspended by vortexing, and the resuspended material was then pelleted by centrifugation at 7500×g for 5 min at 4° C. Finally, the RNA pellet was dried in a Speedvac (Savant, Farmingdale, N.Y.) for 5 min and reconstituted in RNase-free water.

B. RNA Extraction from Blood Mononuclear Cells. Mononuclear cells were isolated from blood samples from patients by centrifugation using Ficoll-Hypaque as follows. A 10 ml volume of whole blood was mixed with an equal volume of RPMI Medium (Gibco BRL, Grand Island, N.Y.). This mixture was then underlayed with 10 ml of Ficoll-Hypaque (Pharmacia, Piscataway, N.J.) and centrifuged for 30 minutes at 200×g. The buffy coat containing the mononuclear cells was removed, diluted to 50 ml with Dulbecco's PBS (Gibco BRL, Grand Island, N.Y.) and the mixture centrifuged for 10 minutes at 200×g. After two washes, the resulting pellet was resuspended in Dulbecco's PBS to a final volume of 1 ml.

RNA was prepared from the isolated mononuclear cells by the TRIZOL® Reagent following the manufacturer's (Gibco BRL, Grand Island, N.Y.) instructions. Briefly, the pelleted cells were resuspended in TRIZOL® Reagent and lysed by repetitive pipetting. The mixture was homogenized and incubated at 15 to 30° C. for 5 minutes. Chloroform was added to the homogenate and the tubes were vigorously shaken by hand for 15 seconds, then incubated at 15 to 30° C. for 2 to 3 minutes. The samples were centrifuged at 12,000×g for 15 minutes at 2 to 8° C. and the aqueous phase containing the RNA was removed to a fresh tube. The RNA was precipitated by mixing with isopropanol, incubated for 10 minutes at 15 to 30° C., and centrifuged at 12,000×g for 10 minutes at 2 to 8° C. The supernatant was removed and the RNA pellet was washed once with 75% ethanol. The pellet was briefly dried and resuspended in RNAse-free water by passing the solution through a pipette tip and incubating for 10 minutes at 55 to 60° C. The RNA was quantitated by UV spectrophotometry.

C. RNA Extraction from polysomes. Tissue is minced in saline at 4° C. and mixed with 2.5 volumes of 0.8 M sucrose in a TK₁₅₀M (150 mM KCl, 5 mM MgCl₂, 50 mM Tris-HCl, pH 7.4) solution containing 6 mM 2-mercaptoethanol. The tissue is homogenized in a Teflon-glass Potter homogenizer with five strokes at 100-200 rpm followed by six strokes in a Dounce homogenizer, as described by B. Mechler, Methods in Enzymology 152: 241-248 (1987). The homogenate then is centrifuged at 12,000×g for 15 min at 4° C. to sediment the nuclei. The polysomes are isolated by mixing 2 ml of the supernatant with 6 ml of 2.5 M sucrose in TK₁₅₀M and layering this mixture over 4 ml of 2.5 M sucrose in TK₁₅₀M in a 38 ml polyallomer tube. Two additional sucrose TK₁₅₀M solutions are successively layered onto the extract fraction; a first layer of 13 ml of 2.05 M sucrose is followed by a second layer of 6 ml of 1.3 M sucrose. The polysomes are isolated by centrifuging the gradient at 90,000×g for 5 h at 4° C. The fraction then is taken from the 1.3 M sucrose/2.05 M sucrose interface with a siliconized Pasteur pipette and diluted in an equal volume of TE (10 mM Tris-HCl, pH 7.4, 1 mM EDTA). An equal volume of 90° C. SDS buffer (1% SDS, 200 mM NaCl, 20 mM Tris-HCl, pH 7.4) is added and the solution is incubated in a boiling water bath for 2 min. Proteins next are digested with a Proteinase K digestion (50 mg/ml) for 15 min at 37° C. The mRNA is purified with 3 equal volumes of phenol-chloroform extractions followed by precipitation with 0.1 volume of 2 M sodium acetate (pH 5.2) and 2 volumes of 100% ethanol at −20° C. overnight. The precipitated RNA is recovered by centrifugation at 12,000×g for 10 min at 4° C. The RNA is dried and resuspended in TE (10 mM Tris-HCl, pH 7.4, 1 mM EDTA) or distilled water. The resuspended RNA then can be used in a slot blot or dot blot hybridization assay to check for the presence of BS106 mRNA (see Example 6).

The quality of nucleic acid and proteins is dependent on the method of preparation used. Each sample may require a different preparation technique to maximize isolation efficiency of the target molecule. These preparation techniques are within the skill of the ordinary artisan.

### Example 4

### Ribonuclease Protection Assay

A. Synthesis of Labeled Complementary RNA (cRNA) Hybridization Probe and Unlabeled Sense Strand. A pINCY plasmid containing the BS106 gene cDNA sequence insert (clone 1662885), flanked by opposed SP6 and T7 polymerase promoters, was purified using Qiagen Plasmid Purification Kit (Qiagen, Chatsworth, Calif.). Then, 10 μg of the plasmid were cut with 10 U Dde I restriction enzyme for 1 h at 37° C. The cut plasmid was purified using QIAprep kits (Qiagen, Chatsworth, Calif.). The purified plasmid was used for the synthesis of antisense transcript labeled with 10 μM (alpha³²P) CTP (800 Ci/mmol)(Amersham Life Sciences, Inc. Arlington Heights, Ill.) starting from the T7 promoter using the Riboprobe® in vitro Transcription System (Promega Corporation, Madison, Wis.), as described by the supplier's instructions. To generate the sense strand, 10 μg of the purified plasmid were cut with restriction enzymes 10 U Xba 1 and 10 U Not I, and 250 ng were transcribed with unlabeled CTP from the SP6 promoter. Both sense and antisense strands were isolated by spin column chromatography. Unlabeled sense strand was quantitated by UV absorption at 260 nm.

B. Hybridization of Labeled Probe to Target. Frozen tissue was pulverized to powder under liquid nitrogen and 100-500 mg were dissolved in lysis buffer available as a component of the RNAqueous™ RNA Isolation Kit (Ambion, Inc., Austin, Tex.). Further dissolution was achieved using a tissue homogenizer and RNA isolated as described by the supplier's instructions. In addition, a dilution series of a known amount of sense strand in yeast RNA was made for use as a positive control. Finally, up to 5.0 μg of purified tissue RNA or diluted sense strand were co-precipitated with 1×10⁵ cpm of radioactively labeled probe (1.5×10⁹ cpm/μg) in ammonium acetate and ethanol. Pellets were air dried and then resuspended in 20 μl of hybridization buffer supplied as a component of the RPA II™ Ribonuclease Protection Assay Kit (Ambion, Inc., Austin, Tex.). After heat denaturation at 90° C. for 5 minutes, hybridization was allowed to proceed overnight at 45° C.

C. RNase Digestion. RNA that was not hybridized to probe was removed from the reaction as per the RPA II™ protocol using a solution of RNase A and RNase T1 for 30 min at 37° C. Hybridized fragments protected from digestion were then precipitated according to the supplier's instructions and by centrifugation at 12,000×g for 15 min.

D. Fragment Analysis. The precipitates were dissolved in denaturing gel loading dye (80% formamide, 10 mM EDTA (pH 8.0), 1 mg/ml xylene cyanol, 1 mg/ml bromophenol blue), heat denatured, and electrophoresed in 6% polyacrylamide TBE, 8 M urea denaturing gels. The gels were imaged and analyzed using the STORM™ storage phosphor autoradiography system (Molecular Dynamics, Sunnyvale, Calif.). Quantitation of protected fragment bands, expressed in femtograms (fg), was achieved by comparing the peak areas obtained from the test samples to those from the known dilutions of the positive control sense strand (see Section B, supra). The results are expressed as an image rating score in Table 1. Samples with no detectable protected fragment were scored “−”; samples with detectable protected fragment, the fg values of which were within the standard curve, were scored “+”.

TABLE 1
RNase Protection Results

| Tissue | Score | Tissue | Score |
|---|---|---|---|
| Placental DNA | − | Malignant Breast | − |
| Normal Breast | − | Malignant Breast | + |
| Normal Breast | + | Malignant Breast | + |
| Normal Breast | + | Malignant Breast | + |
| Normal Breast | + | Normal Lung | − |
| Normal Breast | + | Malignant Lung | − |
| Malignant Breast | − | Normal Colon | − |
| Malignant Breast | − | Malignant Colon | − |

### Example 5

### Northern Blotting

Northern blotting is a well known technique in the art. The Northern blot technique was used to identify a specific size RNA fragment from a complex population of RNA using gel electrophoresis and nucleic acid hybridization. Briefly, 5-10 μg of total RNA (see Example 3) were incubated in 15 μl of a solution containing 40 mM morpholinopropanesulfonic acid (MOPS) (pH 7.0), 10 mM sodium acetate, 1 mM EDTA, 2.2 M formaldehyde, 50% v/v formamide for 15 min at 65° C. The denatured RNA was mixed with 2 μl of loading buffer (50% glycerol, 1 mM EDTA, 0.4% bromophenol blue, 0.4% xylene cyanol) and loaded into a denaturing 1.0% agarose gel containing 40 mM MOPS (pH 7.0), 10 mM sodium acetate, 1 mM EDTA and 2.2 M formaldehyde. The gel was electrophoresed at 60 V for 1.5 h and rinsed in RNAse free water. RNA was transferred from the gel onto nylon membranes (Brightstar-Plus, Ambion, Inc., Austin, Tex.) for 1.5 hours using the downward alkaline capillary transfer method (Chomczynski, Anal. Biochem. 201: 134-139, 1992). The filter was rinsed with 1×SSC, and RNA was crosslinked to the filter using a Stratalinker (Stratagene, Inc., La Jolla, Calif.) on the autocrosslinking mode and dried for 15 min. The membrane was then placed into a hybridization tube containing 20 ml of preheated prehybridization solution (5×SSC, 50% formamide, 5× Denhardt's solution, 100 μg/ml denatured salmon sperm DNA) and incubated in a 42° C. hybridization oven for at least 3 h. While the blot was prehybridizing, a ³²P-labelled random-primed probe was generated using the BS106 insert fragment (obtained by digesting clone 603148H 1 with XbaI and NotI) using Random Primer DNA Labelling System (Life Technologies, Inc., Gaithersburg, Md.) according to the manufacturer's instructions. Half of the probe was boiled for 10 min, quick chilled on ice and added to the hybridization tube. Hybridization was carried out at 42° C. for at least 12 h. The hybridization solution was discarded and the filter was washed in 30 ml of 3×SSC, 0.1% SDS at 42° C. for 15 min, followed by 30 ml of 3×SSC, 0.1% SDS at 42° C. for 15 min. The filter was wrapped in plastic wrap, exposed to Kodak XAR-Omat film for 8-96 h, and the film was developed for analysis.

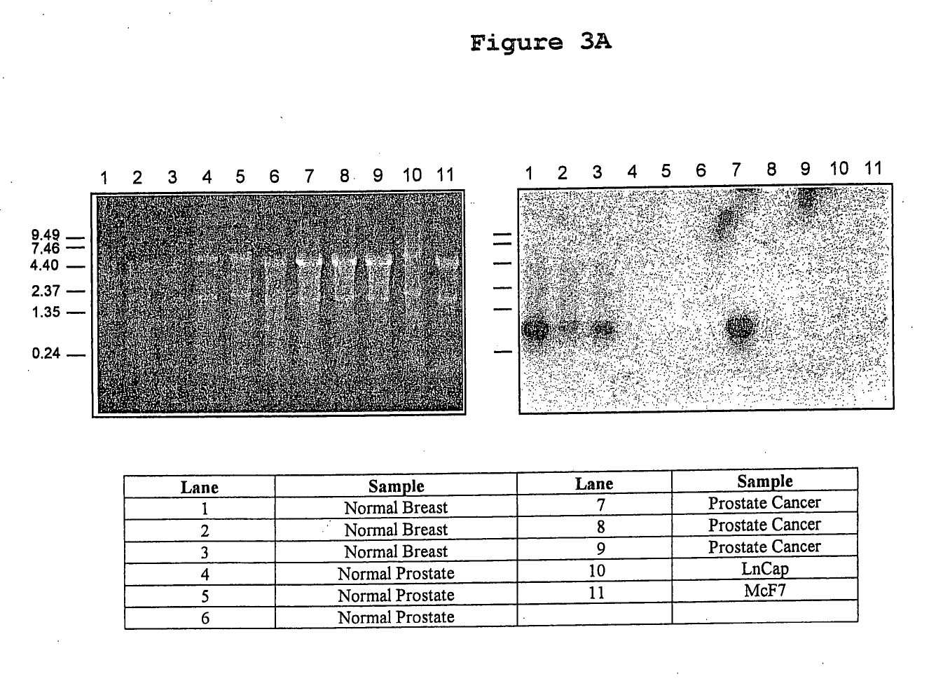

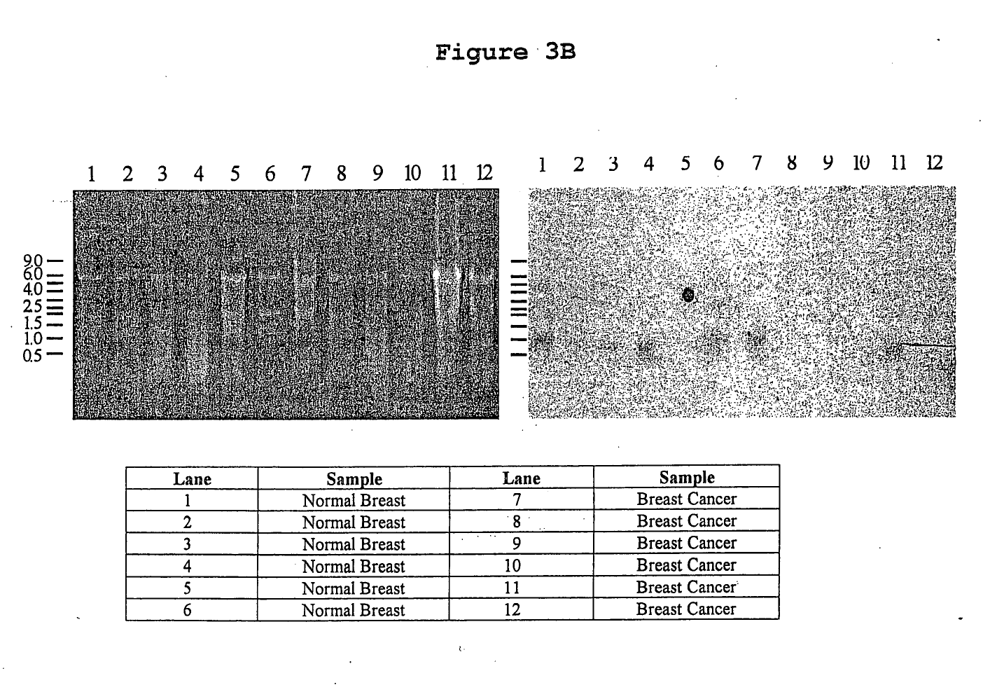

Results of the analysis of RNA quality using an ethidium bromide stained agarose gel and the corresponding northern blot using BS106 probe hybridized to RNAs from breast tissues and prostate tissues, and from normal breast and breast cancer tissues are shown in FIGS. 3A and B, respectively. The positions of RNA size standards (in kb) are shown to the left of each panel. As shown in FIG. 3A, the BS106 probe hybridized to an RNA band of approximately 0.7 kb in 3 of 3 normal breast tissues (lanes 1-3), and in 1 of 3 prostate cancer tissues (lane 7). The RNA band was not detected in 3 normal prostate tissues (lanes 4-6) nor in a prostate or breast cancer cell line (lanes 10 and 11). FIG. 3B shows that the BS106 probe hybridized to an RNA of approximately 0.7 kb in 5 of 6 normal breast RNA samples (lanes 1-4 and 6) and 2 of 6 breast cancer RNA samples (lanes 7 and 11).

Detection of a product comprising a sequence selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof, is indicative of the presence of BS106 mRNA, suggesting a diagnosis of a breast tissue disease or condition, such as breast cancer.

### Example 6

### Dot Blot/Slot Blot

Dot and slot blot assays are quick methods to evaluate the presence of a specific nucleic acid sequence in a complex mix of nucleic acid. Commercially available kits make these assays especially attractive.

A dot blot analysis was performed using a Clontech (Clontech Laboratories, Inc., Palo Alto, Calif.) Multiple Tissue Expression Array™ containing polyA RNA from 76 different human tissues. BS106 plasmid DNA was labeled by nick translation with [³²P]dCTP (Amersham Pharmacia Biotech) to a specific activity of 10⁸ cpm/g. Approximately 63 ng of ³²P-labeled probe was used to hybridize dots on the Clontech array. Hybridization was carried out according to instructions supplied with the Clontech kit. Briefly, labeled probe was hybridized to the expression array membrane at 65° C. for 15 hours. The membrane was washed five times with 2×SSC, 1% SDS at 65° C. followed by two washes with 0.1×SSC, 0.5% SDS at 55° C. The hybridization image was analyzed using the Molecular Dynamics. STORM 840 Phosphor Imaging system™ (Molecular Dynamics, Sunnyvale, Calif.).

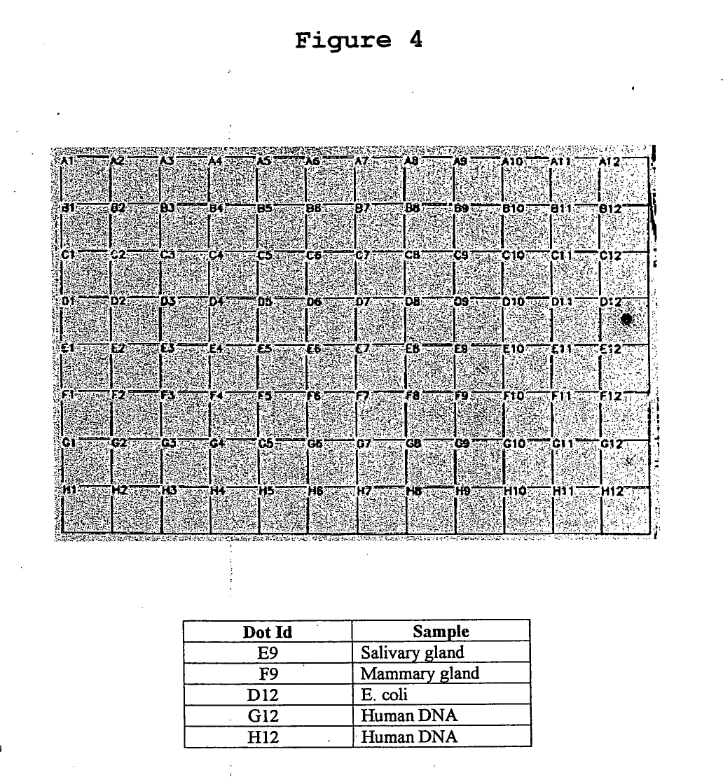

Results of the analysis are shown in FIG. 4. Identification of the dots was provided with the kit. Positive signals were detected with mRNA isolated from human salivary gland and mammary gland and none of the 74 remaining human tissues. Positive signals were also obtained for E. coli DNA and human genomic DNA. Hybridization to the E. coli DNA was expected since the BS106 plasmid was isolated directly from E. coli without any subsequent purification away from residual E. coli DNA. The hybridization to human genomic DNA could indicate that BS106 might belong to a multi-gene family.

### Example 7

### In Situ Hybridization

This method is useful to directly detect specific target nucleic acid sequences in cells using detectable nucleic acid hybridization probes.

Tissues are prepared with cross-linking fixative agents such as paraformaldehyde or glutaraldehyde for maximum cellular RNA retention. See, L. Angerer et al., Methods in Cell Biol. 35: 37-71 (1991). Briefly, the tissue is placed in greater than 5 volumes of 1% glutaraldehyde in 50 mM sodium phosphate, pH 7.5 at 4° C. for 30 min. The solution is changed with fresh glutaraldehyde solution (1% glutaraldehyde in 50 mM sodium phosphate, pH 7.5) for a further 30 min fixing. The fixing solution should have an osmolality of approximately 0.375% NaCl. The tissue is washed once in isotonic NaCl to remove the phosphate.

The fixed tissues then are embedded in paraffin as follows. The tissue is dehydrated though a series of ethanol concentrations for 15 min each: 50% (twice), 70% (twice), 85%, 90% and then 100% (twice). Next, the tissue is soaked in two changes of xylene for 20 min each at room temperature. The tissue is then soaked in two changes of a 1:1 mixture of xylene and paraffin for 20 min each at 60° C.; and then in three final changes of paraffin for 15 min each.

Next, the tissue is cut in 5 μm sections using a standard microtome and placed on a slide previously treated with a tissue adhesive such as 3-aminopropyltriethoxysilane.

Paraffin is removed from the tissue by two 10 min xylene soaks and rehydrated in a series of ethanol concentrations: 99% twice, 95%, 85%, 70%, 50%, 30%, and then distilled water twice. The sections are pre-treated with 0.2 M HCl for 10 min and permeabilized with 2 μg/ml Proteinase-K at 37° C. for 15 min.

Labeled riboprobes transcribed from the BS106 gene plasmid (see Example 4) are hybridized to the prepared tissue sections and incubated overnight at 56° C. in 3× standard saline extract and 50% formamide. Excess probe is removed by washing in 2× standard saline citrate and 50% formamide followed by digestion with 100 μg/ml RNase A at 37° C. for 30 min. Fluorescence probe is visualized by illumination with ultraviolet (UV) light under a microscope. Fluorescence in the cytoplasm is indicative of BS106 mRNA. Alternatively, the sections can be visualized by autoradiography.

### Example 8

### Reverse Transcription PCR

A. One Step RT-PCR Assay. Target-specific primers are designed to detect the above-described target sequences by reverse transcription PCR using methods known in the art. One step RT-PCR is a sequential procedure that performs both RT and PCR in a single reaction mixture. The procedure is performed in a 200 μl reaction mixture containing 50 mM (N,N,-bis[2-Hydroxyethyl]glycine), pH 8.15, 81.7 mM KOAc, 33.33 mM KOH, 0.01 mg/ml bovine serum albumin, 0.1 mM ethylene diaminetetraacetic acid, 0.02 mg/ml NaN₃, 8% w/v glycerol, 150 μM each of dNTP, 0.25 μM each primer, 5U rTth polymerase, 3.25 mM Mn(OAc)₂ and 5 μl of target RNA (see Example 3). Since RNA and the rTth polymerase enzyme are unstable in the presence of Mn(OAc)₂, the Mn(OAc)₂ should be added just before target addition. Optimal conditions for cDNA synthesis and thermal cycling readily can be determined by those skilled in the art. The reaction is incubated in a Perkin-Elmer Thermal Cycler 480. Conditions which may be found useful include cDNA synthesis at 60°-70° C. for 15-45 min and 30-45 amplification cycles at 94° C., 1 min; 55°-70° C., 1 min; 72° C., 2 min. One step RT-PCR also may be performed by using a dual enzyme procedure with Taq polymerase and a reverse transcriptase enzyme, such as MMLV (Moloney murine leukemia virus) or AMV (avian myeloblastosis virus) RT (reverse transcriptase) enzymes.

B. Traditional RT-PCR. Alternatively, a traditional two-step RT-PCR reaction was performed, as described by K. Q. Hu et al., Virology 181: 721-726 (1991). Briefly, 0.5 μg of extracted mRNA (see Example 3) was reverse transcribed in a 20 μl reaction mixture containing 1× PCR II buffer (Perkin-Elmer), 5 mM MgCl₂, 1 mM dNTP, 20 U RNasin, 2.5 μM random hexamers, and 50 U MMLV (Moloney murine leukemia virus) reverse transcriptase (RT). Reverse transcription was performed at 42° C. for 60 min in an MJ Research Cycler Model PTC-200, followed by further incubation at 95° C. for 5 min to inactivate the RT. PCR was performed using 2 μl of the cDNA reaction in a final PCR reaction volume of 50 μl containing 10 mM Tris-HCl (pH 8.3), 50 mM KCl, 2 mM MgCl₂, 200 μM dNTP, 0.5 μM of each sense and antisense primer, SEQUENCE ID NO 13 and SEQUENCE ID NO 14, respectively, and 2.5 U of Taq polymerase. The reaction was incubated in an MJ Research Model PTC-200 as follows: 40 cycles of amplification (94° C., 20 sec; 58° C., 30 sec; 72° C., 30 sec); a final extension (72° C., 10 min); and a soak at 4° C.

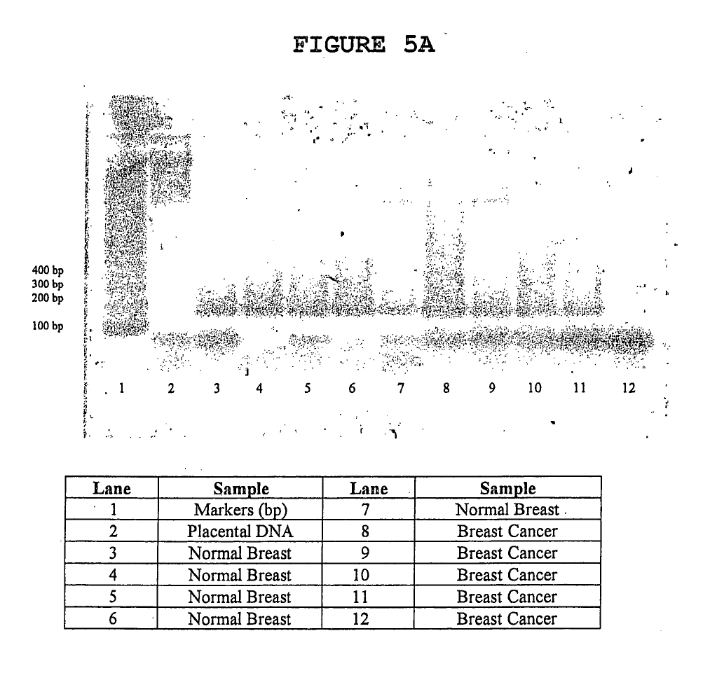

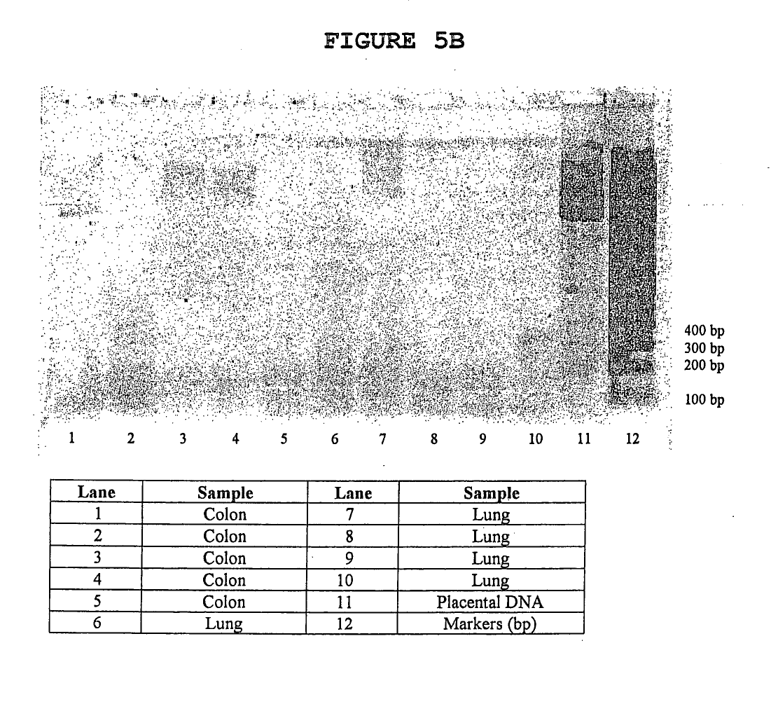

C. PCR Fragment Analysis. The correct products were verified by size determination using gel electrophoresis with a SYBR® Green I nucleic acid gel stain (Molecular Probes, Eugene, Oreg.). Gels were stained with SYBR Green I at a 1:10,000 dilution in 1×TBE for 45 min. Gels were then destained in 1×TBE for 30 min. and imaged using a STORM imaging system (FIGS. 5A and 5B). FIG. 5A shows a DNA band at 201 bases which is indicative of a BS106 mRNA-specific RT-PCR product. This band is present in RNA of 5 of 5 normal breast tissue samples (lanes 3-7) and in RNA of 4 of 5 breast cancer tissue samples (lanes 8-11). It is absent in placental DNA (lane 2). As shown in FIG. 5B, the product was not detected using RNA isolated from colon tissue (lanes 1-5), lung tissue (lanes 6-10), nor in placental DNA (lane 11).

Detection of a product comprising a sequence selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof, is indicative of the presence of BS106 mRNA, suggesting a diagnosis of a breast tissue disease or condition, such as breast cancer.

### Example 9

### OH-PCR

A. Probe selection and Labeling. Target-specific primers (SEQUENCE ID NO 15 and SEQUENCE ID NO 16) and a probe (SEQUENCE ID NO 17) were designed to detect the above described target sequences by oligonucleotide hybridization PCR. International Publication Nos WO 92/10505, published Jun. 25, 1992, and WO 92/11388, published Jul. 9, 1992, teach methods for labeling oligonucleotides at their 5′ and 3′ ends, respectively. The label-phosphoramidite reagent was prepared and used to add the label to the oligonucleotide during its synthesis. [See N. T. Thuong et al., Tet. Letters 29(46): 5905-5908 (1988); J. S. Cohen et al., published U.S. patent application Ser. No. 07/246,688 (NTIS ORDER No. PAT-APPL-7-246,688) (1989)]. Probes were labeled at their 3′ end to prevent participation in PCR and the formation of undesired extension products. For one-step OH-PCR, the probe had a T_{M} at least 15° C. below the T_{M} of the primers. The primers and probes were utilized as specific binding members, with or without detectable labels, using standard phosphoramidite chemistry and/or post-synthetic labeling methods which are well-known to one skilled in the art.

B. One Step Oligo Hybridization PCR. The cDNA was prepared from total RNA (see Example 3) using 2.5 U MMLV Reverse Transcriptase in a buffer of 50 mM KCl, 10 mM Tris-HCl (pH 8.3), 5 mM MgCl₂, 1 mM dGTP, 1 mM dATP, 1 mM dCTP, 1 mM dTTP, 1.0 U RNase Inhibitor, 2.5 μM Oligo d(T) 16 at 42° C. for 30 minutes followed by 99° C. for 5 minutes and 4° C. for 5 minutes. The prepared cDNA was then added to the OH-PCR reaction which contained 190 μl of 50 mM N,N,-bis[2-Hydroxyethyl]glycine (pH 8.15), 81.7 mM KOAc, 33.33 mM KOH, 0.01 mg/ml bovine serum albumin, 0.1 mM ethylene diaminetetraacetic acid, 0.02 mg/ml NaN₃, 8% w/v glycerol, 150 μM each of dNTPs, 0.335 μM forward primer (SEQUENCE ID NO 15), 0.586 μM reverse primer (SEQUENCE ID NO 16), 20.0 nM probe (SEQUENCE ID NO 17), 5U rTth polymerase, and 3.25 mM MnCl₂. The MnCl₂ was added just prior to target addition, since rTth polymerase enzyme is unstable in the presence of MnCl₂. Alternatively, total RNA (see Example 3) can be used as a target instead of cDNA. The reaction was incubated in a Perkin-Elmer Thermal Cycler 480. Optimal conditions for cDNA synthesis and thermal cycling can be readily determined by those skilled in the art. Conditions which were found useful included cDNA synthesis (60° C., 30 min) when starting with RNA, and 37 amplification cycles (94° C., 40 sec; 62° C., 80 sec). Oligo hybridization was performed subsequent to amplification (97° C., 5 min; 12° C., 5 min; 12° C. soak). The correct reaction product contained at least one of the strands of the PCR product and an internally hybridized probe.

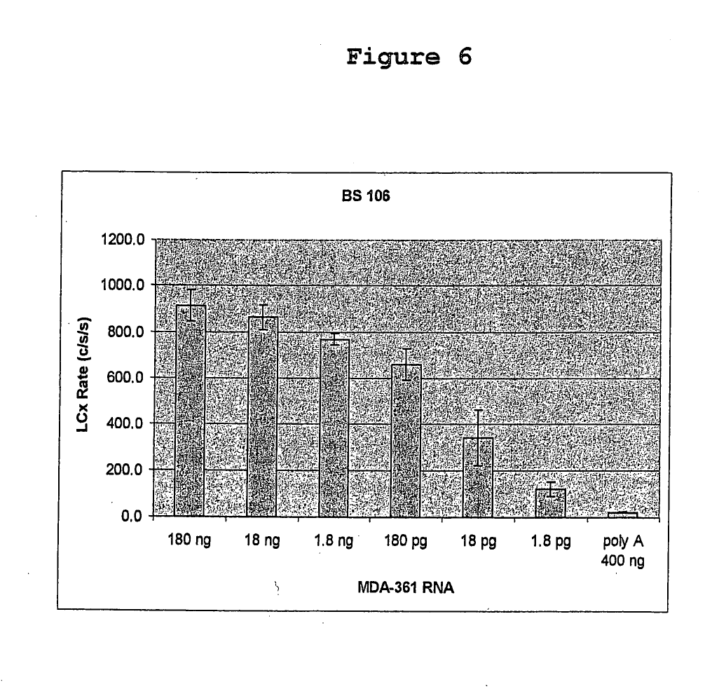

C. OH-PCR Product Analysis. Amplified reaction products were detected on an LCx® Analyzer system (available from Abbott Laboratories, Abbott Park, Ill.). Briefly, the specific reaction product was captured by an antibody labeled microparticle at a capturable site on the hybridization probe and the complex was detected by binding of a detectable antibody conjugate to the PCR product. Only the complex containing a PCR strand hybridized with the internal probe was detectable. FIG. 6 shows the BS106 amplicon was easily detected at 18 picograms of RNA from the MDA 361 cell line. The MDA-361 cell line (#HTB27) originated from a metastatic human breast adenocarcinoma (A.T.C.C., 10801 University Blvd., Manassas, Va.). PolyA RNA was used as a negative control and undetectable even at 400 nanograms.

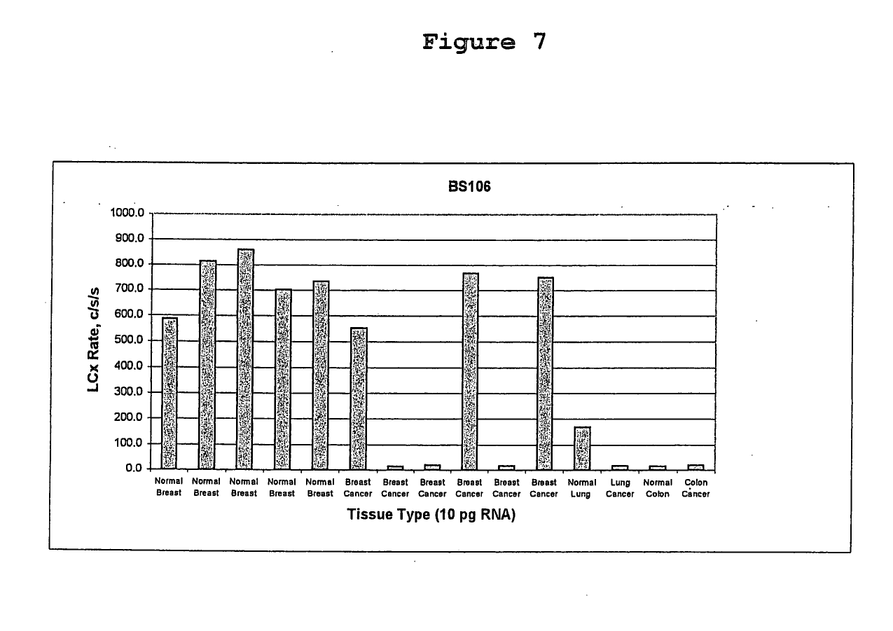

Ten picograms of RNA from various tissues were assayed for BS106 activity using the LCx® system. FIG. 7 shows the overall tissue distribution for the BS106 marker. All 5 normal breast (columns 1-5) and 3 of 6 breast cancer tissue RNAs (columns 6, 9, and 11) were reactive in the BS106 LCx® assay, while lung cancer, normal colon, and colon cancer tissue RNAs (columns 13-15) were non-reactive. Normal lung tissue RNA (column 12) was slightly reactive.

The detection of this complex is indicative of the presence of BS106 mRNA, suggesting a diagnosis of a breast disease or condition, such as breast cancer.

Many other detection formats exist which can be used and/or modified by those skilled in the art to detect the presence of amplified or non-amplified BS106-derived nucleic acid sequences including, but not limited to, ligase chain reaction (LCR, Abbott Laboratories, Abbott Park, Ill.); Q-beta replicase (Gene-Trak™, Naperville, Ill.), branched chain reaction (Chiron, Emeryville, Calif.), and strand displacement assays (Becton Dickinson, Research Triangle Park, N.C.).

### Example 10

### Synthetic Peptide Production

Synthetic peptides were modeled and then prepared based upon the predicted amino acid sequence of the BS106 polypeptide consensus sequence (see Example 1). In particular, a number of BS106 peptides derived from SEQUENCE ID NO 20 were prepared, including the peptides of SEQUENCE ID NOS 21-33. All peptides were synthesized on a Symphony Peptide Synthesizer (available from Rainin Instrument Co, Emeryville Calif.), using FMOC chemistry, standard cycles and in-situ HBTU activation. Cleavage and deprotection conditions were as follows: a volume of 2.5 ml of cleavage reagent (77.5% v/v trifluoroacetic acid, 15% v/v ethanedithiol, 2.5% v/v water, 5% v/v thioanisole, 1-2% w/v phenol) was added to the resin, and agitated at room temperature for 2-4 hours. The filtrate was then removed and the peptide was precipitated from the cleaveage reagent with cold diethyl ether. Each peptide was then filtered, purified via reverse-phase preparative HPLC using a water/acetonitrile/0.1% TFA gradient, and lyophilized. The product was confirmed by mass spectrometry (data not shown).

The purified peptides were mixed with adjuvant, and injected into rabbits (see Example 14). Alternatively, the purified peptides may be conjugated to Keyhole Limpet Hemocyanin with glutaraldehyde, mixed with adjuvant, and injected into rabbits (see Example 14).

### Example 11a

### Expression of Protein in a Cell Line Using Plasmid 577

A. Construction of a BS106 Expression Plasmid. Plasmid 577, described in U.S. patent application Ser. No. 08/478,073, filed Jun. 7, 1995 and incorporated herein by reference, has been constructed for the expression of secreted antigens in a permanent cell line. This plasmid contains the following DNA segments: (a) a 2.3 kb fragment of pBR322 containing bacterial beta-lactamase and origin of DNA replication; (b) a 1.8 kb cassette directing expression of a neomycin resistance gene under control of HSV-1 thymidine kinase promoter and poly-A addition signals; (c) a 1.9 kb cassette directing expression of a dihydrofolate reductase gene under the control of an Simian Virus 40 (SV40) promoter and poly-A addition signals; (d) a 3.5 kb cassette directing expression of a rabbit immunoglobulin heavy chain signal sequence fused to a modified hepatitis C virus (HCV) E2 protein under the control of the Simian Virus 40 T-Ag promoter and transcription enhancer, the hepatitis B virus surface antigen (HBsAg) enhancer I followed by a fragment of Herpes Simplex Virus-1 (HSV-1) genome providing poly-A addition signals; and (e) a residual 0.7 kb fragment of SV40 genome late region of no function in this plasmid. All of the segments of the vector were assembled by standard methods known to those skilled in the art of molecular biology.

Plasmids for the expression of secretable BS106 proteins are constructed by replacing the hepatitis C virus E2 protein coding sequence in plasmid 577 with that of a BS106 polynucleotide sequence selected from the group consisting of SEQUENCE ID NO 1, SEQUENCE ID NO 2, SEQUENCE ID NO 3, SEQUENCE ID NO 4, SEQUENCE ID NO 5, SEQUENCE ID NO 6, and fragments or complements thereof, as follows. Digestion of plasmid 577 with XbaI releases the hepatitis C virus E2 gene fragment. The resulting plasmid backbone allows insertion of the BS106 cDNA insert downstream of the rabbit immunoglobulin heavy chain signal sequence which directs the expressed proteins into the secretory pathway of the cell. The BS106 cDNA fragment is generated by PCR using standard procedures. Encoded in the sense PCR primer sequence is an XbaI site, immediately followed by a 12 nucleotide sequence that encodes the amino acid sequence Ser-Asn-Glu-Leu (“SNEL”) to promote signal protease processing, efficient secretion and final product stability in culture fluids. Immediately following this 12 nucleotide sequence the primer contains nucleotides complementary to template sequences encoding amino acids of the BS106 gene. The antisense primer incorporates a sequence encoding the following eight amino acids just before the stop codons: Asp-Tyr-Lys-Asp-Asp-Asp-Asp-Lys (SEQUENCE ID NO 34). Within this sequence is incorporated a recognition site to aid in analysis and purification of the BS106 protein product. A recognition site (termed “FLAG”) that is recognized by a commercially available monoclonal antibody designated anti-FLAG M2 (Eastman Kodak, Co., New Haven, Conn.) can be utilized, as well as other comparable sequences and their corresponding antibodies. For example, PCR is performed using GeneAmp® reagents obtained from Perkin-Elmer-Cetus, as directed by the supplier's instructions. PCR primers are used at a final concentration of 0.5 μM. PCR is performed on the BS106 plasmid template in a 100 μl reaction for 35 cycles (94° C., 30 seconds; 55° C., 30 seconds; 72° C., 90 seconds) followed by an extension cycle of 72° C. for 10 min.

B. Transfection of Dihydrofolate Reductase Deficient Chinese Hamster Ovary Cells. The plasmid described supra is transfected into CHO/DHFR− cells [DXB-111, Uriacio et al., Proc. Natl. Acad. Sci. USA 77: 4451-4466 (1980)]. These cells are available from the A.T.C.C., 10801 University Blvd., Manassas, Va., under Accession No. CRL 9096. Transfection is carried out using the cationic liposome-mediated procedure described by P. L. Felgner et al., Proc. Natl. Acad. Sci. USA 84: 7413-7417 (1987). Particularly, CHO/DHFR− cells are cultured in Ham's F-12 media supplemented with 10% fetal calf serum, L-glutamine (1 mM) and freshly seeded into a flask at a density of 5-8×10⁵ cells per flask. The cells are grown to a confluency of between 60 and 80% for transfection. Twenty micrograms (20 μg) of plasmid DNA are added to 1.5 ml of Opti-MEM I medium and 100 μl of Lipofectin Reagent (Gibco-BRL; Grand Island, N.Y.) are added to a second 1.5 ml portion of Opti-MEM I media. The two solutions are mixed and incubated at room temperature for 20 min. After the culture medium is removed from the cells, the cells are rinsed 3 times with 5 ml of Opti-MEM I medium. The Opti-MEM I-Lipofection-plasmid DNA solution then is overlaid onto the cells. The cells are incubated for 3 h at 37° C., after which time the Opti-MEM I-Lipofectin-DNA solution is replaced with culture medium for an additional 24 h prior to selection.

C. Selection and Amplification. One day after transfection, cells are passaged 1:3 and incubated with DHFR/G418 selection medium (hereafter, “F-12 minus medium G”). Selection medium is Ham's F-12 with L-glutamine and without hypoxanthine, thymidine and glycine (JRH Biosciences, Lenexa, Kans.) and 300 μg per ml G418 (Gibco-BRL; Grand Island, N.Y.). Media volume-to-surface area ratios of 5 ml per 25 cm² are maintained. After approximately two weeks, DHFRIG418 cells are expanded to allow passage and continuous maintenance in F-12 minus medium G.

Amplification of each of the transfected BS106 cDNA sequences is achieved by stepwise selection of DHFR⁺, G418⁺ cells with methotrexate (reviewed by R. Schimke, Cell 37: 705-713 [1984]). Cells are incubated with F-12 minus medium G containing 150 nM methotrexate (MTX) (Sigma, St. Louis, Mo.) for approximately two weeks until resistant colonies appear. Further gene amplification is achieved by selection of 150 nM adapted cells with 5 μM MTX.

D. Antigen Production. F-12 minus medium G supplemented with 5 μM MTX is overlaid onto just confluent monolayers for 12 to 24 h at 37° C. in 5% CO₂. The growth medium is removed and the cells are rinsed 3 times with Dulbecco's phosphate buffered saline (PBS) (with calcium and magnesium) (Gibco-BRL; Grand Island, N.Y.) to remove the remaining media/serum which may be present. Cells then are incubated with VAS custom medium (VAS custom formulation with L-glutamine with HEPES without phenol red, available from JRH Bioscience; Lenexa, Kans., product number 52-08678P), for 1 h at 37° C. in 5% CO₂. Cells then are overlaid with VAS for production at 5 ml per T flask. Medium is removed after seven days of incubation, retained, and then frozen to await purification with harvests 2, 3 and 4. The monolayers are overlaid with VAS for 3 more seven day harvests.

E. Analysis of Breast Tissue Gene BS106 Antigen Expression. Aliquots of VAS supernatants from the cells expressing the BS106 protein construct are analyzed, either by SDS-polyacrylamide gel electrophoresis (SDS-PAGE) using standard methods and reagents known in the art (Laemmli discontinuous gels), or by mass spectrometry.

F. Purification. Purification of the BS106 protein containing the FLAG sequence is performed by immunoaffinity chromatography using an affinity matrix comprising anti-FLAG M2 monoclonal antibody covalently attached to agarose by hydrazide linkage (Eastman Kodak Co., New Haven, Conn.). Prior to affinity purification, protein in pooled VAS medium harvests from roller bottles is exchanged into 50 mM Tris-HCl (pH 7.5), 150 mM NaCl buffer using a Sephadex G-25 (Pharmacia Biotech Inc., Uppsala, Sweden) column. Protein in this buffer is applied to the anti-FLAG M2 antibody affinity column. Non-binding protein is eluted by washing the column with 50 mM Tris-HCl (pH 7.5), 150 mM NaCl buffer. Bound protein is eluted using an excess of FLAG peptide in 50 mM Tris-HCl (pH 7.5), 150 mM NaCl. The excess FLAG peptide can be removed from the purified BS106 protein by gel electrophoresis or HPLC.

Although plasmid 577 is utilized in this example, it is known to those skilled in the art that other comparable expression systems, such as CMV, can be utilized herein with appropriate modifications in reagent and/or techniques and are within the skill of the ordinary artisan.

The largest cloned insert containing the coding region of the BS106 gene is then sub-cloned into either (i) a eukaryotic expression vector which may contain, for example, a cytomegalovirus (CMV) promoter and/or protein fusible sequences which aid in protein expression and detection, or (ii) a bacterial expression vector containing a superoxide-dismutase (SOD) and CMP-KDO synthetase (CKS) or other protein fusion gene for expression of the protein sequence. Methods and vectors which are useful for the production of polypeptides which contain fusion sequences of SOD are described in EPO 0196056, published Oct. 1, 1986, which is incorporated herein by reference and those containing fusion sequences of CKS are described in EPO Publication No. 0331961, published Sep. 13, 1989, which publication is also incorporated herein by reference. This purified protein can be used in a variety of techniques, including, but not limited to animal immunization studies, solid phase immunoassays, etc.

### Example 11b

### Expression of Protein in a Cell Line Using pcDNA3.1/Myc-His

A. Construction of a BS106 Expression Plasmid. Plasmid pcDNA3.1/Myc-His (Cat.# V855-20, Invitrogen, Carlsbad, Calif.) has been constructed, in the past, for the expression of secreted antigens by most mammalian cell lines. Expressed protein inserts are fused to a myc-his peptide tag. The myc-his tag (SEQUENCE ID NO 35) comprises a c-myc oncoprotein epitope and a polyhistidine sequence which are useful for the purification of an expressed fusion protein by using either anti-myc or anti-his affinity columns, or metalloprotein binding columns.

A plasmid for the expression of secretable BS106 protein was constructed by inserting a BS106 polynucleotide sequence from clone 1662885 (SEQUENCE ID NO 5) into the pcDNA3.1/Myc-His vector. Prior to construction of a BS106 expression plasmid, the BS106 cDNA sequence was first cloned into a pCR®-Blunt vector. The BS106 cDNA fragment was generated by PCR performed using Stratagene® reagents obtained from Stratagene, as directed by the supplier's instructions. PCR primers are used at a final concentration of 0.5 μM. PCR using 5 U of pfu polymerase (Stratagene, La Jolla, Calif.) was performed on the BS106 plasmid template (see Example 2) in a 50 μl reaction for 30 cycles (94° C., 1 min; 65° C., 1.5 min; 72° C., 3 min) followed by an extension cycle of 72° C. for 8 min. The sense PCR primer sequence, SEQUENCE ID NO 13, comprises nucleotides which are identical to the pINCY vector directly upstream of the BS106 gene insert. The antisense primer, SEQUENCE ID NO 14, incorporates a 5′ NotI restriction sequence and a sequence complementary to the 3′ end of the BS106 cDNA insert just upstream of the 3′-most, in-frame stop codon. Five microliters (5 μl) of the resulting blunted-ended PCR product were ligated with 25 ng of linearized pCR®-Blunt vector (Invitrogen, Carlsbad, Calif.) interrupting the lethal ccdB gene of the vector. The resulting ligated vector was transformed into TOP 10 E. coli (Invitrogen, Carlsbad, Calif.) using a One Shot™ transformation kit (Invitrogen, Carlsbad, Calif.) following supplier's directions. The transformed cells were grown on LB-Kan (50 μg/ml kanamycin) selection plates at 37° C. Only cells containing a plasmid with an interrupted ccdB gene grew after transformation (Grant, S. G. N., PNAS 87: 4645-4649 (1990)). Transformed colonies were picked and grown up in 3 ml of LB-Kan broth at 37° C. Plasmid DNA was isolated by using a QIAprep® (Qiagen Inc., Santa Clarita, Calif.) procedure, as directed by the supplier's instructions. The DNA was digested with EcoRI and NotI restriction enzymes to release the BS106 insert fragment. The fragment was electrophoresed on 1% Seakem® LE agarose (FMC, Rockland, Me.)/0.5 μg/ml ethidium bromide/TE gel, visualized by UV illumination, excised and purified using QIAquick™ (Qiagen Inc., Santa Clarita, Calif.) procedures, as directed by the supplier's instructions.

The pcDNA3.1/Myc-His plasmid DNA was linearized by digestion with EcoRI and NotI, sites present in the polylinker region of the plasmid DNA. The BS106 purified fragment, supra, was ligated with the resulting plasmid DNA downstream from a CMV promoter, and transformed into DH5 alphat™ cells (GibcoBRL Gaithersburg, Md.), as directed by the supplier's instructions. Briefly, 10 ng of pcDNA3.1/Myc-His containing the BS106 insert were added to 50 μl of competent DH5 alpha cells, and the contents were mixed gently. The mixture was incubated on ice for 30 min, heated for 20 sec at 37° C., and placed on ice for an additional 2 min. Upon addition of 0.95 ml of LB medium, the mixture was incubated for 1 h at 37° C. while shaking at 225 rpm. The transformed cells then were plated onto 100 mm LB/Amp (50 μg/ml ampicillin) plates and grown at 37° C. Colonies were picked and grown in 3 ml of LB/ampicillin broth. Plasmid DNA was purified using a QIAprep kit. The presence of the insert was confirmed using restriction enzyme digestion and gel analysis (J. Sambrook et al., supra.).

B. Transfection of Human Embryonic Kidney 293 Cells. The BS106 expression plasmid described in Section A, supra, was retransformed into DH5 alpha cells, plated onto LB/ampicillin agar, and grown in 10 ml of LB/ampicillin broth, as described hereinabove. The plasmid was purified using a QIAfilter™ Maxi kit (Qiagen, Chatsworth, Calif.) and transfected into HEK293 cells (F. L. Graham et al., J. Gen. Vir. 36: 59-72 (1977)). These cells are available from the A.T.C.C., 10801 University Blvd., Manassas, Va., under Accession No. CRL 1573. Transfection was carried out using the cationic lipofectamine-mediated procedure described by P. Hawley-Nelson et al., Focus 15.73 (1993). Particularly, HEK293 cells were cultured in 10 ml DMEM media, supplemented with 10% fetal bovine serum (FBS), L-glutamine (2 mM), and freshly seeded into 12×100 mm culture plates at a density of 8×10⁶ cells per plate. The cells were grown at 37° C. to a confluency of between 70% and 80% for transfection. Eight micrograms (8 μg) of plasmid DNA were added to 800 μl of Opti-MEM I® medium (Gibco-BRL, Grand Island, N.Y.), and 48-96 μl of Lipofectamine™ Reagent (Gibco-BRL, Grand Island, N.Y.) were added to a second 800 μl portion of Opti-MEM I® medium. The two solutions were mixed and incubated at room temperature for 15-30 min. After the culture medium was removed from the cells, the cells were washed once with 10 ml of serum-free DMEM. The Opti-MEM I-Lipofectamine-plasmid DNA solution was diluted with 6.4 ml of serum-free DMEM and then overlaid onto the cells. The cells were incubated for 5 h at 37° C., after which time an additional 8 ml of DMEM with 20% FBS were added. After 18-24 h, the old medium was aspirated, and the cells were overlaid with 5 ml of fresh DMEM with 5% FBS. Supernatants and cell extracts were analyzed for BS106 gene activity 72 h after transfection.

C. Production of stable cell line. HEK293-106C1. The BS106 expression plasmid described in section A, supra, was retransformed into DH5 alpha cells, plated onto LB/ampicillin agar, and grown up in 10 ml of LB/ampicillin broth. The plasmids were purified using a QIAfilter™ Maxi Kit (Qiagen, Chatsworth, Calif.) and were transfected into HEK293 cells [F. L. Graham et al., J. Gen. Vir. 36: 59-72 (1977)].

The purified expression plasmid, as described supra, was transfected into HEK293 cells [F. L. Graham et al., J. Gen. Vir. 36: 59-72 (1977)]. These cells are available from the A.T.C.C., 10801 University Blvd., Manassas, Va., under Accession No. CRL 1573. Transfection of the expression plasmid was performed using the cationic lipofectamine-mediated procedure described by P. Hawley-Nelson et al., Focus 15.73 (1993). Particularly, HEK293 cells were cultured in 10 ml DMEM media supplemented with 10% fetal bovine serum (FBS), L-glutamine (2 mM), sodium pyruvate (1 mM) and essential amino acids and freshly seeded into 60 mm culture plates at a density of 9×10⁶ cells per plate. The cells were grown at 37° C. to a confluency of between 70% and 80% for transfection. Two micrograms (2 μg) of plasmid DNA were added to 800 μl of unsupplemented DMEM medium (Gibco-BRL, Grand Island, N.Y.). Eight microliters (8 μl) of Plus Reagent (Gibco-BRL, Grand Island, N.Y.) were added to this solution, which was then mixed briefly. Twelve microliters (12 μl) of Lipofectamine (LTI) were added to a second 800 μl portion of unsupplemented DMEM media. After a 15 minute incubation, the two solutions were mixed and incubated at room temperature for an additional 15-30 minutes. During this time the culture medium was removed from the plates containing the HEK293 cells. The DMEM containing the Plus reagent:Lipofectamine:plasmid DNA complex was then overlaid onto the cells. The cells were incubated for 5 h at 37° C. and 5% CO₂, after which time, an additional 2-8 ml of DMEM with 20% FBS were added. After 18-24 h, the old medium was aspirated, and the cells were overlaid with 5 ml of fresh DMEM with 5% FBS containing 400 μg/ml G418, and the incubation was continued until 72 h had elapsed. Supernatants were analyzed for BS106 M/H (Myc/His) polypeptide expression by Western blot analysis.

At 72 hours post transfection, the cells were released from the dish by limited trypsinization and reseeded into 100 mm culture dishes in DMEM, 10% FBS, and 400 μg/ml G418 at dilutions of 1:100, 1:1000 and 1:10000. These cultures were allowed to grow for 5-7 days, until well-isolated foci of cells were identified by microscopy. These foci were isolated by cloning cylinders, their cells released by limited trypsinization, and individual foci were transferred to separate wells in 24-well dishes, again in DMEM, 10% FBS, 400 μg/ml G418. After growth for 7-10 days, the supernatants of each well were analysed for BS106 M/H expression by Western blot analysis, as described hereinbelow. The clonal line labeled 106C1 was found to express BS106 M/H in the supernatant. This line was expanded into 75 cm² flasks, and then passaged 1:30 three times, following expression of BS106 M/H to ensure stability of the insertion event. The final product of this procedure was a cell line derived from HEK293 cell line that expresses BS106 M/H, which we have labeled 106C1 (HEK293-106C1).

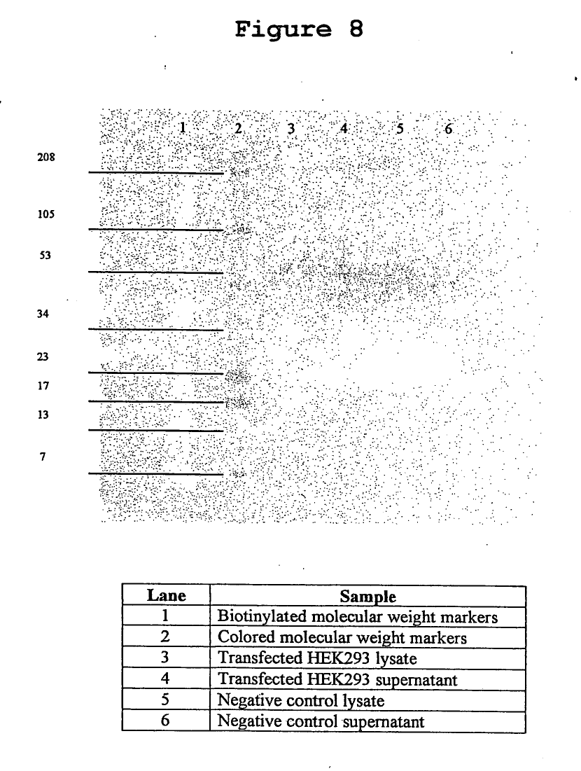

D. Analysis of Breast Tissue Gene BS106 Anti gen Expression. The culture supernatant, (Example 11b. Subsection B), was transferred to cryotubes and stored on ice. HEK293 cells were harvested by washing twice with 10 ml of cold Dulbecco's PBS and lysing by addition of 1.5 ml of CAT lysis buffer (Boehringer Mannheim, Indianapolis, Ind.), followed by incubation for 30 min at room temperature. Lysate was transferred to 1.7 ml polypropylene microfuge tubes and centrifuged at 1000×g for 10 min. The supernatant was transferred to new cryotubes and stored on ice. Aliquots of supernatants from the cells and the lysate of the cells expressing the BS106 protein construct were analyzed for the presence of BS106 recombinant protein. The aliquots were run on SDS-polyacrylamide gel electrophoresis (SDS-PAGE) using standard methods and reagents known in the art (J. Sambrook et al., supra). These gels were then blotted onto nitrocellulose, and the BS106 protein band was visualized using Western blotting techniques with an anti-myc epitope monoclonal antibody (Invitrogen, Carlsbad, Calif.). FIG. 8 shows the resultant Western blot. Lane 1 contains biotinylated molecular weight markers. Lane 2 contains colored molecular weight markers. Lane 3 contains the cell lysate of transiently transfected HEK293 cells. Lane 4 contains the supernatant from the transiently transfected HEK293 cells. Lane 5 contains the cell lysate of a negative control (HEK293 cells not transiently transfected). Lane 6 contains the supernatant of a negative control (HEK293 cells not transiently transfected). Lane 4 shows BS106 M/H as a broad band at approximately 40 kD. Lane 3 shows BS106 M/H as a sharp band at approximately 17 kD. The difference in molecular weight between the two samples is attributed to glycosylation. Specifically, BS106 M/H in lane 3 is found inside the cell, which has yet to be fully post-translationally modified. The BS106 M/H of lane 4, which has been secreted from the cell, is a more mature, more fully modified protein. Heterogeneity in glycosylation results in molecular weight differences among the molecules contributing to the broadness of the band observed in lane 4.

E. Purification. Purification of the BS106 recombinant protein containing the myc-his sequence was performed using Chelating Sepharose Fast Flow (Pharmacia Biotech, Piscataway, N.J.) charged with nickel which specifically binds polyhistidine residues. Three hundred fifty milliliters (350 ml) of supernatant from the transiently transfected HEK293 cells, prepared as described in subsection B, were pooled and passed over the 40 ml nickel-charged column. Non-binding protein was eluted by washing the column with 10 mM Tris-HCl (pH 7.4)/500 mM NaCl buffer. Bound BS106 recombinant protein then was eluted from the column using an imidazole gradient. The flow rate was 2 ml/min; the gradient was 3.1 mM imidazole per milliliter of buffer; and the elution time was 80 minutes, creating an elution profile that went from 0 to 500 mM imidazole.

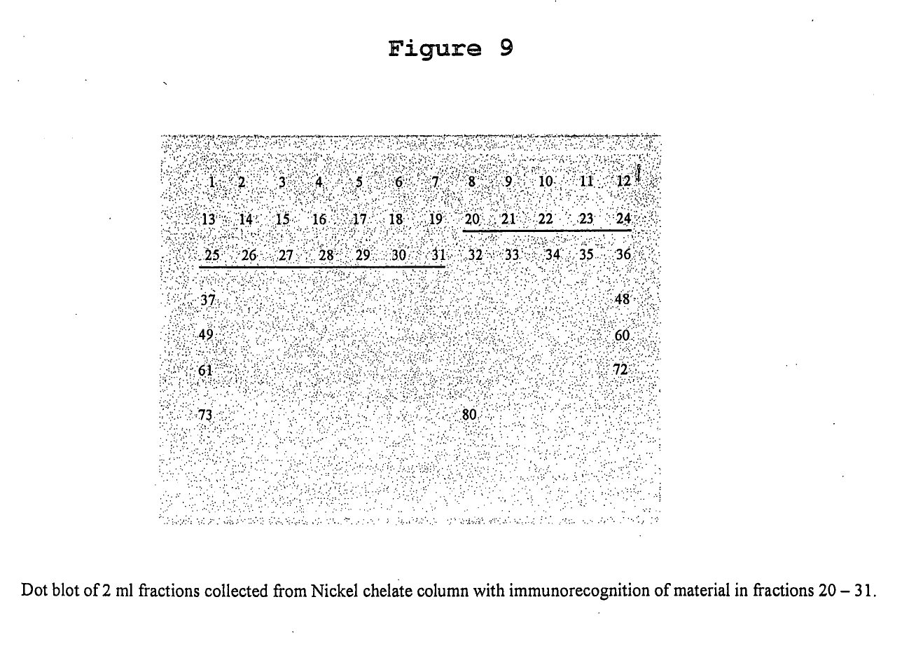

Each 2 ml fraction was sampled. One hundred microliters of each fraction was applied to a well of a dot blot apparatus and the volume was suctioned through a piece of nitrocellulose. The nitrocellulose filter was then developed with the same procedure to develop Western blots, as described supra, using a monoclonal antibody recognizing a myc epitope. FIG. 9 illustrates the developed dot blot, which shows immunorecognition of material in fractions 20-31 by the anti-myc monoclonal antibody. These fractions correspond to elution conditions of 125-200 mM imidazole indicating the successful binding of the histidine tagged proteins to the nickel column and their elution with a histidine analogue. Fractions 20-31 were pooled and dialysed for a minimum of 4 hours each, against 2×4 l of phosphate buffered saline (PBS, 50 mM phosphate, 150 mM sodium chloride, pH 7.4) using Slide-a-Lysers (3500 MWCO).

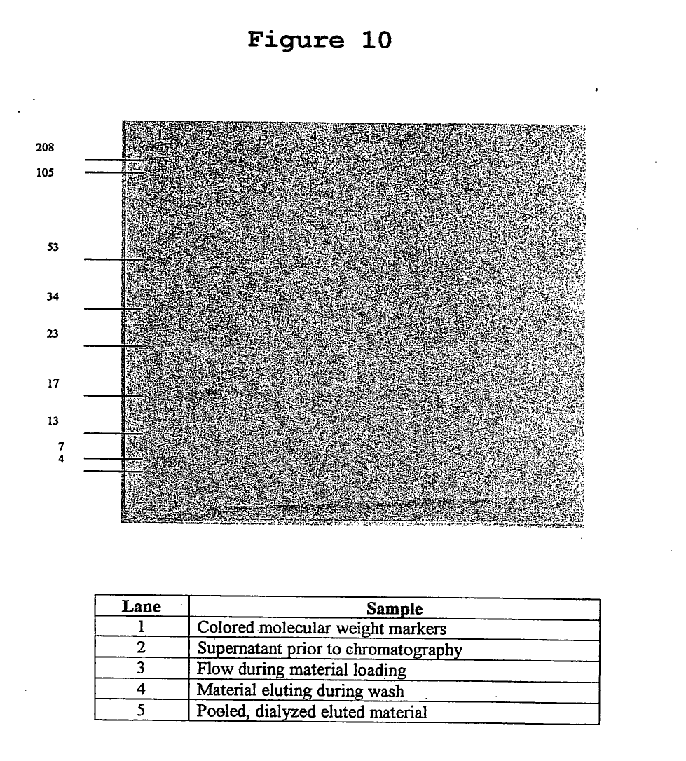

The pooled, dialyzed, semi-purified supernatant was analyzed for the presence of BS106 M/H recombinant protein by Western blot, as described in subsection D. FIG. 10 illustrates the blot where lane 1 contains colored molecular weight markers; lane 2 contains the supernatant prior to chromatography; lane 3 contains the flow through material during sample loading; lane 4 contains the material eluting during column washing; and lane 5 contains the pooled, dialyzed, eluted material. BS106 M/H appeared in lane 5 (and faintly in lane 2) as a broad band with an apparent molecular weight of 40 kD, higher than the theoretical molecular weight of the peptide backbone of 10.7 kD. The added molecular mass of the secreted protein product is attributed to glycosylation.

In a similar manner, 400 ml of supernatant from the growth of the 106C1 cells were purified using nickel chelation chromatography. Briefly, a 1.6 cm×20 cm Hi-Trap Chelate column (Pharmacia Biotech, Piscataway, N.J.) was charged with 50 ml of nickel chloride and equilibrated with buffer, 15 mM Tris (pH 7.5)/450 mM NaCl. NaCl (300 mM) was added to the crude supernatant prior to application to the column. After sample application, the column was washed with buffer containing 25 mM imidazole. The myc/his tagged protein was then eluted with buffer containing 300 mM imidazole. Eight milliliter (8 ml) fractions were collected. These-fractions were tested for the presence of the myc epitope by Western blot, and those fractions found to be positive were pooled.

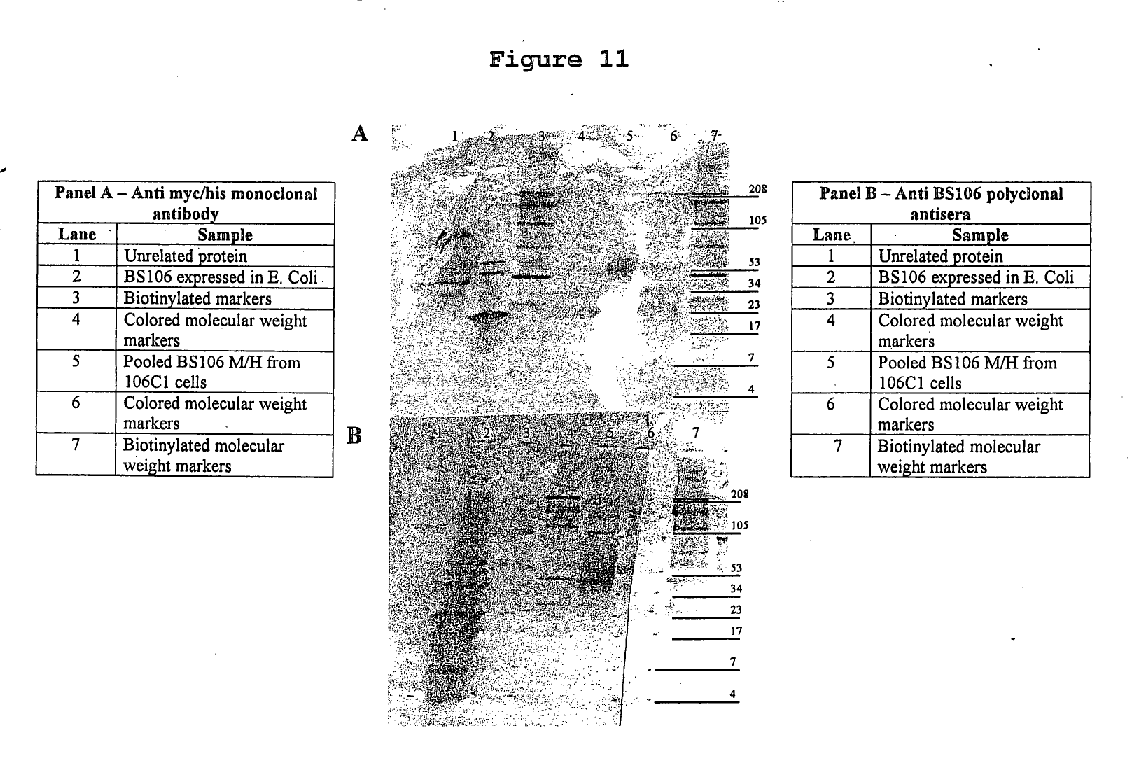

The pooled, purified BS106 M/H was analysed by Western blot using both an anti-myc monoclonal antibody (FIG. 11 panel A) and an anti-BS106 polyclonal antisera (FIG. 11 panel B). In panels A and B, lane 1 contains an unrelated protein; lane 2 contains BS106 expressed in E. coli lane 3 contains biotinylated molecular markers; lane 4 contains colored molecular markers; lane 5 contains the pooled, purified BS106 M/H from 106C1 cells; lane 6 contains colored molecular markers; and lane 7 contains biotinylated molecular markers. BS106 M/H from the 106C1 cells was recognized by both anti-myc monoclonal antibody (lane 5, panel A) and the anti-BS106 polyclonal antisera (lane 5, panel B) and had an apparent molecular weight of 40 kD, consistent with the material provided by the transient transfection of HEK293 cells.

F. Coating Microtiter Plates with BS106 Expressed Proteins. Supernatant from a 100 mm plate, as described supra, is diluted in an appropriate volume of PBS. Then, 100 μl of the resulting mixture is placed into each well of a Reacti-Bind™ metal chelate microtiter plate (Pierce, Rockford, Ill.), incubated at room temperature while shaking, and followed by three washes with 200 μl each of PBS with 0.05% Tween® 20. The prepared microtiter plate can then be used to screen polyclonal antisera for the presence of BS106 antibodies (see Example 17).

Although pcDNA3.1/Myc-His is utilized in this example, it is known to those skilled in the art that other comparable expression systems can be utilized herein with appropriate modifications in reagent and/or techniques and are within the skill of one of ordinary skill in the art. The largest cloned insert containing the coding region of the BS106 gene is sub-cloned into either (i) a eukaryotic expression vector which may contain, for example, a cytomegalovirus (CMV) promoter and/or protein fusible sequences which aid in protein expression and detection, or (ii) a bacterial expression vector containing a superoxide-dismutase (SOD) and CMP-KDO synthetase (CKS) or other protein fusion genes for expression of the protein sequence. Methods and vectors which are useful for the production of polypeptides which contain fusion sequences of SOD are described in published EPO application No. EP 0 196 056, published Oct. 1, 1986, which is incorporated herein by reference, and vectors containing fusion sequences of CKS are described in published EPO application No. EP 0 331 961, published Sep. 13, 1989, which publication is also incorporated herein by reference. The purified protein can be used in a variety of techniques, including but not limited to, animal immunization studies, solid phase immunoassays, etc.

### Example 11c

### Expression of Protein in a Cell Line Using pcDNA3.1

A. Construction of a BS106 Expression Plasmid. Plasmid pcDNA3.1 (Cat.# V790-20, Invitrogen, Carlsbad, Calif.) has been used, in the past, for the expression of secreted antigens by most mammalian cell lines. A plasmid for the expression of secretable BS106 protein was constructed by inserting the BS106 polynucleotide sequence from clone 1662885inh into the pcDNA3.1 vector. In order to construct the BS106 expression plasmid, the BS106 cDNA fragment was excised with EcoRI and NotI restriction enzymes. The fragment was electrophoresed on 1% Seakem® LE agarose (FMC, Rockland, Me.)/0.5 μg/ml ethidium bromide/TE gel, visualized by UV illumination, excised and purified using QIAquick™ (Qiagen Inc., Santa Clarita, Calif.) procedures, as directed by the supplier's instructions.

The pcDNA3.1 plasmid DNA was linearized by digestion with EcoRI and NotI, sites present in the polylinker region of the plasmid DNA. The BS106 purified fragment was ligated with the resulting plasmid DNA and transformed into DH5 alpha™ cells (GibcoBRL Gaithersburg, Md.), as directed by the supplier's instructions. Briefly, 10 ng of pcDNA3.1 containing the BS106 insert were added to 50 μl of competent DH5 alpha cells, and the contents were mixed gently. The mixture was incubated on ice for 30 min, heated for 20 sec at 37° C., and placed on ice for an additional 2 min. Upon addition of 0.95 ml of LB medium, the mixture was incubated for 1 h at 37° C. while shaking at 225 rpm. The transformed cells then were plated onto 100 mm LB/Amp (50 μg/ml ampicillin) plates and grown at 37° C. Colonies were picked and grown in 3 ml of LB/ampicillin broth. Plasmid DNA was purified using a QIAprep kit. The presence of the insert was confirmed using restriction enzyme digestion and gel analysis (J. Sambrook et al., supra.).

B. Transfection of Human Embryonic Kidney Cell 293 Cells. The BS106 expression plasmid described in section A, supra, was retransformed into DH5 alpha cells, plated onto LB/ampicillin agar, and grown up in 100 ml of LB/ampicillin broth, as described hereinabove. The plasmid was purified using a QIAfilter™ Maxi Kit (Qiagen, Chatsworth, Calif.) and was transfected into HEK293 cells [F. L. Graham et al., J. Gen. Vir. 36: 59-72 (1977)]. These cells were available from the A.T.C.C., 10801 University Boulevard, Manassas, Va. 20110-2209, under Accession No. CRL 1573. Transfection was carried out using the cationic lipofectamine-mediated procedure [P. Hawley-Nelson et al., Focus 15.73 (1993)]. In particular, HEK293 cells were cultured in 10 ml DMEM media supplemented with 10% fetal bovine serum (FBS), L-glutamine (2 mM) and freshly seeded into 100 mm culture plates at a density of 2×10⁶ cells per plate for 72 hour incubation or 7×10⁶ cells per plate for 24 hour incubation. The cells were grown at 37° C. to a confluency of between 70% and 80% for transfection. Eight micrograms (8 μg) of plasmid DNA were added to 800 μl of Opti-MEM I® medium (Gibco-BRL, Grand Island, N.Y.), and 48 μl of Lipofectamine™ Reagent (Gibco-BRL, Grand Island, N.Y.) were added to a second 800 μl portion of Opti-MEM I media. The two solutions were mixed gently and incubated at room temperature for 15-30 min. After the culture medium was removed from the cells, the cells were washed once with 10 ml of serum-free DMEM. The Opti-MEM I-Lipofectamine-plasmid DNA solution was diluted with 6.4 ml of serum-free DMEM and then overlaid onto the cells. The cells were incubated for 5 h at 37° C., after which time, an additional 8 ml of DMEM with 20% FBS were added. After 18-24 h, the old medium was aspirated, and the cells were overlaid with 5 ml of fresh DMEM with 5% FBS. Supernatants and cell extracts were analyzed for BS106 gene activity 72 h after transfection.

C. Analysis of Breast Tissue Gene BS106 Antigen Expression. The culture supernatant, supra, is transferred to cryotubes and stored on ice. HEK293 cells are harvested by washing twice with 10 ml of cold Dulbecco's PBS and lysed by addition of 1.5 ml of CAT lysis buffer (Boehringer Mannheim, Indianapolis, Ind.), followed by incubation for 30 min at room temperature. Lysate is transferred to 1.7 ml polypropylene microfuge tubes and centrifuged at 1000×g for 10 min. The supernatant is transferred to new cryotubes and stored on ice. Aliquots of supernatants from the cells and the lysate of the cells expressing the BS106 protein construct are analyzed for the presence of BS106 recombinant protein. The aliquots can be electrophoresed on SDS-polyacrylamide gels (SDS-PAGE) using standard methods and reagents known in the art. (J. Sambrook et al., supra) These gels can then be blotted onto a solid medium such as nitrocellulose, nytran, etc., and the BS106 protein band can be visualized using-Western blotting techniques with anti-myc epitope or anti-histidine monoclonal antibodies (Invitrogen, Carlsbad, Calif.) or anti-BS106 polyclonal serum (see Example 14). Alternatively, the expressed BS106 recombinant protein can be analyzed by mass spectrometry (see Example 12).

### Example 11d

### Expression of Protein in a Cell Line Using pGEX4T/Myc-His

A. Construction of a BS106 Expression Plasmid. A plasmid suitable for the production of protein BS106 as a fusion protein in E. coli was produced. The fusion protein partner was glutathione-5-transferase, and the vector was designed so that the fused protein could be released by limited proteolysis with thrombin. All cloning modifications were made with the method of J. Sambrook et al, supra.

Plasmid pG4-BS106 was constructed from the BS106 polynucleotide sequence [clone 1662885inh (SEQUENCE ID NO 5)] from the pcDNA 3.1 myc/his vector and the bacterial expression vector pGEX4T (Invitrogen, Carlsbad, Calif.). The BS106 polynucleotide sequence [clone 1662885inh (SEQUENCE ID NO 5)] including the myc/his tag was obtained from the pcDNA3.1/Myc-His vector construct (as described in Example 11b subsection A). The BamH I restriction site was inserted into the BS106 pcDNA 3.1 myc/his vector construct to separate the coding sequence of the protein signal sequence and the coding sequence of the mature protein. The BS106 polynucleotide sequence [clone 1662885inh (SEQUENCE ID NO 5)] including the myc/his tag was excised from this modified vector construct with BamH I and Pme I.

The pGEX4T vector (Invitrogen, Carlsbad, Calif.) was modified by inserting a Pme I site using oligonucleotide primers. The BS106 polynucleotide sequence [clone 1662885inh (SEQUENCE ID NO 5)] including the myc/his tag was cloned into the BamH I/Pme I sites of the modified pGEX4T vector. The BamH I site was then removed using oligonucleotides of SEQUENCE ID NO 36 to restore a native amino acid sequence to the released fusion protein product. The resultant pG4-BS106 vector was transformed into DH5 alpha™ cells (GibcoBRL Gaithersburg, Md.), as directed by the supplier's instructions.

B. Expression of GST-BS106 M/H. Four liters of Super Broth media (24 g/l yeast extract, 12 g/l tryptone, 5 g/l glycerol, 100 mM K phosphate pH 7.4) containing 100 μg/ml ampicillin were inoculated with 200 ml LB media in which E coli DH5 alpha cells containing vector pG4-106 had been grown to stationary phase. This mixture was then allowed to grow with aeration (10 lpm air) and agitation (300 rpm) at 37° C. for 3 h. IPTG was added to 100 μM, and growth continued for another 3 h. Cells (˜100 g) were harvested by centrifugation (˜5000×g), and lysed by sonication (45 min) on ice in 250 ml 1% Triton X-100 in phosphate buffered saline. After cellular debris was pelleted by centrifugation, the supernatant containing the expressed protein from the cellular lysate was saved.

C. Purification of BS106 M/H. The lysate supernatant was passed over a glutathione-agarose column (50 ml). The column was washed with phosphate buffered saline until the baseline as detected by absorbance at 280 nm was stable (˜300 ml). The GST-BS106 M/H fusion protein was then eluted with 0.15% glutathione in phosphate buffered saline.

This material was further purified using a Mono Q 10/10 column (Pharmacia, Piscataway, N.J.) with 25 mM Tris buffer pH 8.8 and a linear gradient of NaCl from 0-1000 mM. Fractions eluting between 200 to 300 mM NaCl were found to contain full-length fusion protein as detected by Western blotting using a myc specific monoclonal antibody. These fractions were pooled and treated with bovine thrombin in a ratio of 50 U thrombin per mole fusion protein (calculated using an A_{280 1%} ˜1, and a molecular mass ˜50 kD) for 4 h at 37° C. PMSF was added to 1 mM, and the sample was again purified by ion exchange chromatography under identical conditions. The BS106 M/H eluted at approximately 100 mM NaCl. The fractions were pooled and dialyzed against phosphate buffered saline.

This preparation was subjected to Western blot analysis using both myc specific and BS106 specific antibodies. Lane 2 of FIG. 11, panels A and B, which was described in Example 11b, subsection E, demonstrates the material in this pool. The anti-myc monoclonal antibody (panel A) recognized a major band at approximately 13-20 kD as well as two other higher molecular weight species. The anti-BS106 polyclonal antibody recognized more bands. The extra bands are attributed to some fusion protein missing the myc-his tag (since these bands were not detected in the myc specific blot), non-fusion protein missing the myc-his tag, and some degradation products from thrombin cleavage.

### Example 12

### Chemical Analysis of Breast Tissue Proteins

A. Analysis of Tryptic Peptide Fragments Using MS. Sera from patients with breast disease, such as breast cancer, sera from patients with no breast disease, extracts of breast tissues or cells from patients with breast disease, such as breast cancer, extracts of breast tissues or cells from patients with no breast disease, and extracts of tissues or cells from other non-diseased or diseased organs of patients are run on a polyacrylamide gel using standard procedures and stained with Coomassie Blue. Sections of the gel suspected of containing the unknown polypeptide are excised and subjected to an in-gel reduction, acetamidation and tryptic digestion. P. Jeno et al., Anal. Bio. 224: 451-455 (1995) and J. Rosenfeld et al., Anal. Bio. 203: 173-179 (1992). The gel sections are washed with 100 mM NH₄HCO₃ and acetonitrile. The shrunken gel pieces are swollen in digestion buffer (50 mM NH₄HCO₃, 5 mM CaCl₂ and 12.5 μg/ml trypsin) at 4° C. for 45 min. The supernatant is aspirated and replaced with 5 to 10 μl of digestion buffer without trypsin and allowed to incubate overnight at 37° C. Peptides are extracted with 3 changes of 5% formic acid and acetonitrile and evaporated to dryness. The peptides are adsorbed to approximately 0.1 μl of POROS R2 sorbent (Perseptive Biosystems, Framingham, Mass.) trapped in the tip of a drawn gas chromatography capillary tube by dissolving them in 10 μl of 5% formic acid and passing it through the capillary. The adsorbed peptides are washed with water and eluted with 5% formic acid in 60% methanol. The eluant is passed directly into the spraying capillary of an API III mass spectrometer (Perkin-Elmer Sciex, Thornhill, Ontario, Canada) for analysis by nano-electrospray mass spectrometry. M. Wilm et al., Int. J. Mass Spectrom. Ion Process 136: 167-180 (1994) and M. Wilm et al., Anal. Chem. 66: 1-8 (1994). The masses of the tryptic peptides are determined from the mass spectrum obtained off the first quadrupole. Masses corresponding to predicted peptides can be further analyzed in MS/MS mode to give the amino acid sequence of the peptide.

B. Peptide Fragment Analysis Using LC/MS. The presence of polypeptides predicted from mRNA sequences found in hyperplastic disease tissues also can be confirmed using liquid chromatography/tandem mass spectrometry (LC/MS/MS). D. Hess et al., METHODS, A Companion to Methods in Enzymology 6: 227-238 (1994). The serum specimen or tumor extract from the patient is denatured with SDS and reduced with dithiothreitol (1.5 mg/ml) for 30 min at 90° C. followed by alkylation with iodoacetamide (4 mg/ml) for 15 min at 25° C. Following acrylamide electrophoresis, the polypeptides are electroblotted to a cationic membrane and stained with Coomassie Blue. Following staining, the membranes are washed and sections thought to contain the unknown polypeptides are cut out and dissected into small pieces. The membranes are placed in 500 μl microcentrifuge tubes and immersed in 10 to 20 μl of proteolytic digestion buffer (100 mM Tris-HCl, pH 8.2, containing 0.1 M NaCl, 10% acetonitrile, 2 mM CaCl₂ and 5 μg/ml trypsin) (Sigma, St. Louis, Mo.). After 15 h at 37° C., 3 μl of saturated urea and 1 μl of 100 μg/ml trypsin are added and incubated for an additional 5 h at 37° C. The digestion mixture is acidified with 3 μl of 10% trifluoroacetic acid and centrifuged to separate supernatant from membrane. The supernatant is injected directly onto a microbore, reverse phase HPLC column and eluted with a linear gradient of acetonitrile in 0.05% trifluoroacetic acid. The eluate is fed directly into an electrospray mass spectrometer, after passing though a stream splitter if necessary to adjust the volume of material. The data is analyzed following the procedures set forth in Example 12, Section A.

### Example 13

### Gene Immunization Protocol

A. In Vivo Antigen Expression. Gene immunization circumvents protein purification steps by directly expressing an antigen in vivo after inoculation of the appropriate expression vector. Also, production of antigen by this method may allow correct protein folding and glycosylation since the protein is produced in mammalian tissue. The method utilizes insertion of the gene sequence into a plasmid which contains a CMV promoter, expansion and purification of the plasmid and injection of the plasmid DNA into the muscle tissue of an animal. Preferred animals include mice and rabbits. See, for example, H. Davis et al., Human Molecular Genetics 2: 1847-1851 (1993). After one or two booster immunizations, the animal can then be bled, ascites fluid collected, or the animal's spleen can be harvested for production of hybridomas.

B. Plasmid Preparation and Purification. BS106 cDNA sequences are generated from the BS106 cDNA-containing vector using appropriate PCR primers containing suitable 5′ restriction sites following the procedures described in Example 11. The PCR product is cut with appropriate restriction enzymes and inserted into a vector which contains the CMV promoter (for example, pRc/CMV or pcDNA3 vectors from Invitrogen, San Diego, Calif.). This plasmid then is expanded in the appropriate bacterial strain and purified from the cell lysate using a CsCl gradient or a Qiagen plasmid DNA purification column. All these techniques are familiar to one of ordinary skill in the art of molecular biology.

C. Immunization Protocol. Anesthetized animals are immunized intramuscularly with 0.1-100 μg of the purified plasmid diluted in PBS or other DNA uptake enhancers (Cardiotoxin, 25% sucrose). See, for example, H. Davis et al., Human Gene Therapy 4: 733-740 (1993); and P. W. Wolff et al., Biotechniques 11: 474-485 (1991). One to two booster injections are given at monthly intervals.

D. Testing and Use of Antiserum. Animals are bled and the resultant sera tested for antibody using peptides synthesized from the known gene sequence (see Example 16) using techniques known in the art, such as Western blotting or EIA techniques. Antisera produced by this method can then be used to detect the presence of the antigen in a patient's tissue or cell extract or in a patient's serum by ELISA or Western blotting techniques, such as those described in Examples 15 through 18.

### Example 14

### Production of Antibodies Against BS106

A. Production of Polyclonal Antisera. Antiserum against BS106 was prepared by injecting rabbits with peptides whose sequences were derived from that of the predicted amino acid sequence of the BS106 consensus nucleotide sequence (SEQUENCE ID NO 6). The synthesis of peptides (SEQUENCE ID NO 21-33) is described in Example 10. Peptides used as immunogens were either conjugated to a carrier protein such as keyhole limpet hemocyanine, KLH, or not conjugated to a carrier (i.e., they were unconjugated.).

1. Peptide Conjugation. Peptide was conjugated to maleimide activated keyhole limpet hemocyanine (KLH, commercially available as Imject®, available from Pierce Chemical Company, Rockford, Ill.). Imject® contains about 250 moles of reactive maleimide groups per mole of hemocyanine. The activated KLH was dissolved in phosphate buffered saline (PBS, pH 8.4) at a concentration of about 7.7 mg/ml. The peptides were conjugated through cysteines occurring in the peptide sequence, or to a cysteine previously added to the synthesized peptide in order to provide a point of attachment. The peptides were dissolved in dimethyl sulfoxide (DMSO, Sigma Chemical Company, St. Louis, Mo.) and reacted with the activated KLH at a mole ratio of about 1.5 moles of peptide per mole of reactive maleimide attached to the KLH. A procedure for the conjugation of peptides SEQUENCE ID NO 23, SEQUENCE ID NO 25, SEQUENCE ID NO 26, and SEQUENCE ID NOS 28′-32 is provided hereinbelow. It is known to the ordinary artisan that the amounts, times and conditions of such a procedure can be varied to optimize peptide conjugation.

The conjugation reaction described hereinbelow was based on obtaining 3 mg of KLH peptide conjugate (“conjugated peptide”), which contains about 0.77 μmoles of reactive maleimide groups. This quantity of peptide conjugate usually was adequate for one primary injection and four booster injections for production of polyclonal antisera in a rabbit. Briefly, the peptides (SEQUENCE ID NO 23, SEQUENCE ID NO 25, SEQUENCE ID NO 26, and SEQUENCE ID NOS 28-32) were dissolved in DMSO at a concentration of 1.16 μmoles/100 μl of DMSO. One hundred microliters (100 μl) of the DMSO solution was added to 380 μl of the activated KLH solution prepared as described hereinabove, and 20 μl of PBS (pH 8.4) was added to bring the volume to 500 μl. The reaction was incubated overnight at room temperature with stirring. The extent of reaction was determined by measuring the amount of unreacted thiol in the reaction mixture. The difference between the starting concentration of thiol and the final concentration was assumed to be the concentration of peptide which had coupled to the activated KLH. The amount of remaining thiol was measured using Ellman's reagent (5,5′-dithiobis(2-nitrobenzoic acid), Pierce Chemical Company, Rockford, Ill.). Cysteine standards were made at a concentration of 0, 0.1, 0.5, 2, 5 and 20 mM by dissolving 35 mg of cysteine HCl (Pierce Chemical Company, Rockford, Ill.) in 10 ml of PBS (pH 7.2) and diluting the stock solution to the desired concentration(s). The photometric determination of the concentration of thiol was accomplished by placing 200 μl of PBS (pH 8.4) in each well of an Immulon 2® microwell plate (Dynex Technologies, Chantilly, Va.). Next, 10 μl of standard or reaction mixture was added to each well. Finally, 20 μl of Ellman's reagent at a concentration of 1 mg/ml in PBS (pH 8.4) was added to each well. The wells were incubated for 10 minutes at room temperature, and the absorbance of all wells was read at 415 nm with a microplate reader (such as the BioRad Model 3550, BioRad, Richmond, Calif.). The absorbance of the standards was used to construct a standard curve and the thiol concentration of the reaction mixture was determined from the standard curve. A decrease in the concentration of free thiol was indicative of a successful conjugation reaction. Unreacted peptide was removed by dialysis against PBS (pH 7.2) at room temperature for 6 hours. The conjugate was stored at 2-8° C. if it is to be used immediately; otherwise, it was stored at −20° C. or colder.

2. Animal Immunization. Female white New Zealand rabbits weighing 2 kg or more were used for raising polyclonal antiserum. One animal was immunized per conjugated peptide (SEQUENCE ID NO 23, SEQUENCE ID NO 25, SEQUENCE ID NO 26, and SEQUENCE ID NOS 28-32) or unconjugated peptide (SEQUENCE ID NOS 21-24). One week prior to the first immunization, 5 to 10 ml of blood were obtained from the animal to serve as a non-immune prebleed sample.

Conjugated, (SEQUENCE ID NO 23, SEQUENCE ID NO 25, SEQUENCE ID NO 26, and SEQUENCE ID NOS 28-32), or unconjugated peptides, (SEQUENCE ID NOS 21-24), were used to prepare the primary immunogen by emulsifying 0.5 ml of the conjugated or unconjugated peptide at a concentration of 2 mg/ml in PBS (pH 7.2) which contained 0.5 ml of complete Freund's adjuvant (CFA) (Difco, Detroit, Mich.). The immunogen was injected into several sites of the animal via subcutaneous, intraperitoneal, and intramuscular routes of administration. Four weeks following the primary immunization, a booster immunization was administered. The immunogen used for the booster immunization dose was prepared by emulsifying 0.5 ml of the same conjugated or unconjugated peptide used for the primary immunogen, except that the peptide or peptide/carrier complex now was diluted to 1 mg/ml with 0.5 ml of incomplete Freund's adjuvant (IFA) (Difco, Detroit, Mich.). Again, the booster dose was administered into several sites via subcutaneous, intraperitoneal and intramuscular types of injections. The animals were bled (5 ml) two weeks after the booster immunizations and each serum was tested for immunoreactivity to the peptide as described below. The booster and bleed schedule was repeated at 4 week intervals until an adequate titer was obtained. The titer or concentration of antiserum was determined by using unconjugated peptides in a microtiter EIA as described in Example 17, below. An antibody titer of 1:500 or greater was considered an adequate titer for further use and study. Table 2 below shows the titers obtained with the peptide immunized rabbits.

TABLE 2
Titer of rabbit anti-BS106 peptide antisera (12 week bleed)

| | Peptide Conjugated to a | |
|---|---|---|
| Peptide Immunogen | Carrier Protein? | Titer |
| SEQUENCE ID NO 21 | no | 200 |
| SEQUENCE ID NO 22 | no | 5000 |
| SEQUENCE ID NO 23 | no | 44,000 |
| SEQUENCE ID NO 23 | yes | >62,500 |
| SEQUENCE ID NO 24 | no | <100 |
| SEQUENCE ID NO 25 | yes | 62,500 |
| SEQUENCE ID NO 26 | yes | 258,000 |
| SEQUENCE ID NO 28* | yes | 39,000 |
| SEQUENCE ID NO 29 | yes | 47,000 |
| SEQUENCE ID NO 30 | yes | 200 |
| SEQUENCE ID NO 31 | yes | 1000 |
| SEQUENCE ID NO 32 | yes | 400 |

*Rabbit antisera to BS106.8 was tested using SEQUENCE ID NO 29 peptide

B. Production of Monoclonal Antibody.

1. Immunization Protocol. Mice were immunized using peptides conjugated (SEQUENCE ID NO 23, SEQUENCE ID NO 26, and SEQUENCE ID NO 29) to a carrier such as KLH [prepared as described hereinabove] except that the amount of the conjugated peptide for monoclonal antibody production in mice was one-tenth the amount used to produce polyclonal antisera in rabbits. Thus, the primary immunogen consisted of 100 μg of conjugated peptide in 0.1 ml of CFA emulsion while the immunogen used for booster immunizations consisted of 50 μg conjugated peptide in 0.1 ml of IFA. Hybridomas for the generation of monoclonal antibodies were prepared and screened using standard techniques. The methods used for monoclonal antibody development followed procedures known in the art such as those detailed in Kohler and Milstein, Nature 256: 494 (1975) and reviewed in J. G. R. Hurrel, ed., Monoclonal Hybridoma Antibodies: Techniques and Applications, CRC Press, Inc., Boca Raton, Fla. (1982). Another method of monoclonal antibody development which is based on the Kohler and Milstein method is that of L. T. Mimms et al., Virology 176: 604-619 (1990), which is incorporated herein by reference.

The immunization regimen (per mouse) consisted of a primary immunization with additional booster immunizations. The primary immunogen used for the primary immunization consisted of 100 μg of conjugated peptide in 50 μl of PBS (pH 7.2) previously emulsified in 50 μl of CFA. Booster immunizations performed at approximately two weeks and four weeks post primary immunization consisted of 50 μg of conjugated peptide in 50 μl of PBS (pH 7.2) emulsified with 50 μl IFA. A total of 100 μl of this immunogen was inoculated intraperitoneally and subcutaneously into each mouse. Individual mice were screened for immune response by microtiter plate enzyme immunoassay (EIA) as described in Example 17 approximately four weeks after the third immunization. Mice were inoculated intravenously with 50 μg of conjugated peptide in PBS (pH 7.2) approximately fifteen weeks after the third immunization.

Three days after this intravenous boost, splenocytes were fused with Sp2/0-Ag 14 myeloma cells (Milstein Laboratories, England) using the polyethylene glycol (PEG) method. The fusions were cultured in Dulbecco's Modified Eagle's Medium (DMEM) with the addition of L-glutamine, L-asparagine, L-arginine, folic acid, and containing 10% fetal calf serum (FCS), plus 1% hypoxanthine, aminopterin and thymidine (HAT). Bulk cultures were screened by microtiter plate EIA following the protocol in Example 17. Clones reactive with the peptide used an immunogen and non-reactive with other peptides (i.e., peptides of BS106 not used as the immunogen) were selected for final expansion. Supernatant from the final expansion was harvested and used for further characterization. Hybridoma cells from the expansion growth were harvested, aliquoted and frozen in DMEM containing 10% FCS and 10% dimethyl sulfoxide, (DMSO).

2. Production of Ascites Fluid Containing Monoclonal Antibodies. Frozen hybridoma cells prepared as described hereinabove are thawed and placed into expansion culture. Viable hybridoma cells are inoculated intraperitoneally into Pristane treated mice. Ascitic fluid is removed from the mice, pooled, filtered through a 0.2μ filter and subjected to an immunoglobulin class G (IgG) analysis to determine the volume of the Protein A column required for the purification.

3. Purification of Monoclonal Antibodies From Ascites Fluid or Cell Culture Supernatant. Monoclonal antibodies can be purified from ascites fluid or cell culture supernatant using a variety of methods including Protein A, Protein G, and Protein L column chromatography or precipitation. Briefly, filtered and thawed ascites fluid is mixed with an equal volume of Protein A sepharose binding buffer (1.5 M glycine, 3.0 M NaCl, pH 8.9) and refiltered through a 0.2μ filter. The volume of the Protein A column is determined by the quantity of IgG present in the ascites fluid. The eluate then is dialyzed against PBS (pH 7.2) overnight at 2-8° C. The dialyzed monoclonal antibody is sterile filtered and dispensed in aliquots. The immunoreactivity of the purified monoclonal antibody is confirmed by determining its ability to specifically bind to the peptide used as the immunogen by use of the EIA microtiter plate assay procedure of Example 17. The specificity of the purified monoclonal antibody is confirmed by determining its lack of binding to irrelevant peptides such as peptides of BS106 not used as the immunogen. The purified anti-BS106 monoclonal antibody thus prepared and characterized is placed at either 2-8° C. for short term storage or at −80° C. for long term storage.

4. Further Characterization of Monoclonal Antibody. The isotype and subtype of the monoclonal antibody produced as described hereinabove were determined using an EIA microtiter assay. Briefly, the peptide immunogen was prepared at 2 μg/ml in 50 mM carbonate, pH 9.6 and 100 μl was placed in each well of an Immunlon 2 High Binding microtiter plate (Dynex Technologies, Chantilly, Va.). The plate was incubated for 14-18 hours at room temperature and then washed four times with deionized water. The wells were blocked by adding 125 μl of Superblock (Pierce Chemical Company, Rockford, Ill.) to each well and then immediately discarding the solution. This blocking procedure was performed three times. One hundred microliters (100 μl) of culture supernatant was diluted 1:2 in a protein blocking agent of PBS containing 3% Superblock, 0.05% Tween 20 (monolaurate polyoxyethlene ether) (Sigma Chemical Company, St. Louis, Mo.), and 0.05% sodium azide and placed in each well of the coated microtiter plate. The wells were incubated for one hour at room temperature. Each well was washed four times with deionized water. One hundred microliters (100 μl) of alkaline phosphatase-conjugated goat anti-mouse IgG (H+L), or IgG1, or IgG2a, or IgG2b, or IgG3 (Southern Biotech, Birmingham, Ala.) diluted 1:2000 in PBS containing 3% Superblock, 0.05% Tween 20, and 0.05% sodium azide were added to the appropriate wells. The wells were incubated for one hour at room temperature. Next, each well was washed four times with deionized water. One hundred microliters (100 μl) of para-nitrophenyl phosphate substrate (Kirkegaard and Perry Laboratories, Gaithersburg, Md.) then were added to each well. The wells were incubated for thirty minutes at room temperature and the absorbance at 405 nm was read. The results of the isotyping are presented in Table 3.

TABLE 3
Characterization of Monoclonal Antibodies

| Study | Experiment | Hybridoma | Peptide Immunogen | Isotype |
|---|---|---|---|---|
| 392 | 3 | H6C52 | SEQUENCE ID NO 26 | IgG1 |
| 392 | 3 | H9C29 | SEQUENCE ID NO 26 | IgG1 |
| 392 | 3 | H39C51 | SEQUENCE ID NO 26 | IgG1 |
| 392 | 3 | H80C32 | SEQUENCE ID NO 26 | IgG1 |
| 392 | 7 | H84C55 | SEQUENCE ID NO 26 | IgG1 |
| 392 | 7 | H121C68 | SEQUENCE ID NO 26 | IgG1 |
| 392 | 7 | H144C20 | SEQUENCE ID NO 26 | IgG2a |
| 392 | 7 | H24C16 | SEQUENCE ID NO 26 | IgG1 |
| 392 | 14 | H17C77 | SEQUENCE ID NO 23 | IgG1 |
| 392 | 14 | H59C34 | SEQUENCE ID NO 23 | IgG1 |
| 392 | 14 | H121C19 | SEQUENCE ID NO 23 | IgG1 |
| 392 | 14 | H131C50 | SEQUENCE ID NO 23 | IgG1 |
| 392 | 14 | H147C64 | SEQUENCE ID NO 23 | ND |
| 392 | 14 | H184C51 | SEQUENCE ID NO 23 | IgG1 |
| 392 | 14 | H195C62 | SEQUENCE ID NO 23 | IgG1 |
| 392 | 14 | H212C68 | SEQUENCE ID NO 23 | IgG1 |
| 392 | 14 | H218C31 | SEQUENCE ID NO 23 | IgG2a |
| 392 | 15 | H119C17 | SEQUENCE ID NO 26 | IgG1 |
| 392 | 15 | H150C31 | SEQUENCE ID NO 26 | IgG1 |
| 392 | 15 | H182C63 | SEQUENCE ID NO 26 | IgG2b |
| 392 | 15 | H188C82 | SEQUENCE ID NO 26 | IgG1 |
| 392 | 15 | H203C46 | SEQUENCE ID NO 26 | IgG2a |
| 392 | 15 | H205C33 | SEQUENCE ID NO 26 | IgG1 |
| 400 | 3 | H24C29 | SEQUENCE ID NO 29 | IgG1 |
| 400 | 3 | H51C42 | SEQUENCE ID NO 29 | IgG3 |
| 400 | 3 | H62C52 | SEQUENCE ID NO 29 | IgG2b |
| 400 | 3 | H102C47 | SEQUENCE ID NO 29 | ND |
| 400 | 3 | H30C35 | SEQUENCE ID NO 29 | ND |
| 400 | 3 | H26C39 | SEQUENCE ID NO 29 | ND |
| 400 | 4 | H16C70 | SEQUENCE ID NO 29 | IgG1 |
| 400 | 4 | H45C35 | SEQUENCE ID NO 29 | IgG1 |
| 400 | 4 | H62C59 | SEQUENCE ID NO 29 | IgG1 |
| 400 | 4 | H76C43 | SEQUENCE ID NO 29 | ND |
| 400 | 4 | H4C33 | SEQUENCE ID NO 29 | ND |
| 400 | 4 | H57C43 | SEQUENCE ID NO 29 | ND |
| 400 | 4 | H44C56 | SEQUENCE ID NO 29 | ND |
| 400 | 4 | H94C45 | SEQUENCE ID NO 29 | ND |
| 400 | 4 | H34C78 | SEQUENCE ID NO 29 | ND |
| 400 | 4 | H2C15 | SEQUENCE ID NO 29 | ND |

Stability testing also can be performed on the monoclonal antibody by placing an aliquot of the monoclonal antibody in continuous storage at 2-8° C. and assaying optical density (OD) readings throughout the course of a given period of time.

C. Use of Recombinant Proteins as Immunogens. It is within the scope of the present invention that recombinant proteins made as described herein can be utilized as immunogens in the production of polyclonal and monoclonal antibodies, with corresponding changes in reagents and techniques known to those skilled in the art.

### Example 15

### Purification of Serum Antibodies Which Specifically Bind to BS106 Peptides

A. Preparation of Affinity Column for Purification of Rabbit Anti-BS106. Immune sera, obtained as described hereinabove in Example 14, was affinity purified using an immobilized synthetic peptide prepared as described in Example 10. SEQUENCE ID NO 26 was coupled to SulfoLink™ gel (Pierce Chemical, Rockford, Ill.) according to the manufacturer's instructions. Briefly, 2.0 ml peptide, dissolved in 2.0 ml coupling buffer was poured into 2 ml SulfoLink™ gel previously washed with 10 ml coupling buffer. After 18 h at room temperature the gel was washed with 4 ml coupling buffer, then blocked with 10 mg L-cysteine hydrochloride in 2.0 ml coupling buffer. After 30 min the gel was washed with 8 ml coupling buffer, then 7 ml 0.2% BSA in PBS, then 7 ml PBS. The gel was conditioned by washing with 7 ml of 100 mM glycine pH 2.08, then 15 ml PBS.

B. Affinity Purification of Rabbit Anti-BS106 Antisera. Ten milliliters (10 ml) of rabbit anti-BS106 antiserum was passed through a column containing 2 ml of the affinity gel, as described supra. The gel was washed with PBS until the absorbance at 280 nm was less than 0.05, then the bound antibody was eluted with 100 mM glycine pH 2.08. The eluted material was collected as 500 μl fractions in tubes containing 30 μl of 1.0 M Tris base. Fractions 4-9 were combined and passed through a 10 ml gel filtration column (Pierce Kwik™ column), exchanging the buffer to PBS. Fractions collected from this column were pooled into a 3.0 ml volume at 3.0 mg/ml and a 1.5 ml volume at 0.32 mg/ml.

### Example 16

### Western Blotting of Tissue Samples

A. Tissue Specificity. Tissue extracts were prepared by homogenizing tissue samples in 0.1M Tris-HCl (pH 7.5), 15% (w/v) glycerol, 0.2 mM EDTA, 1.0 mM 1,4-dithiothreitol, 10 μg/ml leupeptin and 1.0 mM phenylmethylsulfonylfluoride (Kain et al., Biotechniques, 17: 982 (1994). The homogenates were centrifuged at 4° C. for 5 minutes to separate supernate from debris. For protein quantitation, 3-10 μL of supernate were added to 1.5 ml of bicinchoninic acid reagent (Sigma, St. Louis, Mo.), and the resulting absorbance at 562 nm was measured.

For SDS-PAGE, samples were adjusted to desired protein concentration with Tricine Buffer (Novex, San Diego, Calif.), mixed with an equal volume of 2× Tricine sample buffer (Novex, San Diego, Calif.), and heated for 5 minutes at 100° C. in a thermal cycler. Samples were then applied to a Novex 10-20% Precast Tricine Gel for electrophoresis. Following electrophoresis, samples were transferred from the gels to nitrocellulose membranes in Novex Tris-Glycine Transfer buffer. Membranes were then probed with Protein G purified anti-peptide monoclonal antibody H9C29 at 1 μg/ml. The blots were then incubated with commercially obtained anti-mouse alkaline phosphatase (Tropix, Bedford, Mass.). The bands were visualized directly on the membranes by the addition of 5-bromo-4-chloro-3-indolyl phosphate (BCIP). This chromogenic solution contains 0.016% BCIP in a solution containing 100 mM NaCl, 5 mM MgCl₂ and 100 mM Tris-HCl (pH 9.5). Molecular mass determination was preformed using pre-stained molecular weight standards (Novex, San Diego, Calif.).

Competition experiments were also carried out in an analogous manner as above, with the following exception; the primary antibody (anti-peptide monoclonal antibody) was pre-incubated overnight in the refrigerator with 1.3 μM, 0.43 μM and 0.14 μM of BS106 peptide SEQUENCE ID NO 26 prior to exposure to the nitrocellulose membrane.

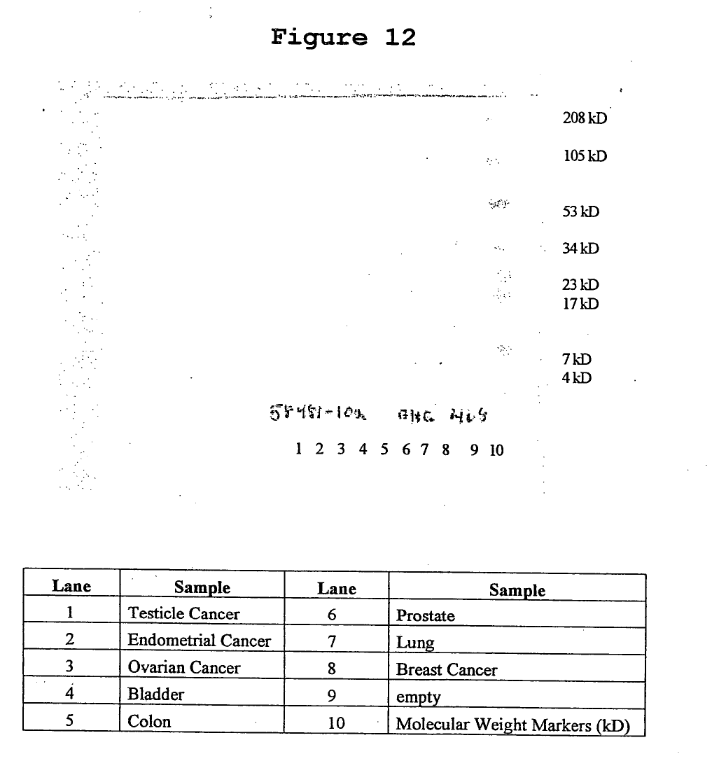

FIG. 12 shows the results of a Western blot performed on a panel of tissue extracts using a monoclonal antibody (H9C29) directed against BS106 peptide SEQUENCE ID NO 26. Each lane of FIG. 12 contains a different tissue extract: 1, testicle cancer; 2, endometrial cancer; 3, ovarian cancer; 4, bladder; 5, colon; 6, prostate; 7, lung; 8, breast cancer; 9, blank and 10 molecular weight markers (kD). The extract from the breast cancer tissue (lane 8) shows a strong broad band consistent with a glycoprotein at approximately 40 kD, whereas the other tissues are negative. Several other breast tissue specimens were analyzed including extracts form seven other breast cancer tissues and six normals tissues. Together, five of the eight breast cancers and two of the six normals were positive for BS106. Competition experiments with a BS106 peptide (SEQUENCE ID NO 26) resulted in complete inhibition of the band seen with breast cancer tissue.

B. Analysis of Human Milk. Aqueous and fat portions of a sample of human milk, separated as in Example 22D, were prepared for electrophoresis as indicated in the Table 4 below. Milk fat was diluted with an equal volume of PBS prior to preparation for electrophoresis. The samples were incubated for 10 minutes at room temperature after addition of the TCEP (Tris-carboxyethylphosphine hydrochloride). PBS and 2×SDS Sample Buffer (Novex, San Diego, Calif.) were then added. The mixtures were heated 5 minutes at 100° C., then sodium carbonate was added to neutralize the TCEP acidity. Twelve microliters (12 μl) of the prepared samples were added to the wells of a 10-20% Tris-glycine gel (Novex, San Diego, Calif.). The samples were run in duplicate with molecular weight markers between the two sets of samples. After electrophoresis was complete, the gels were blotted to nitrocellulose, as described in Example 16A. The nitrocellulose filters were blocked for 60 minutes with Iblock™ (Tropix, Bedford Mass.) (0.2% in PBS containing 0.1% Tween-20). The blot was then cut to separate the replicated samples. Both blots (labeled A and B) were incubated for 60 minutes with 10 ml of Iblock™ solution containing 2.5 μl of affinity purified rabbit anti-BS106.6. However, the B blot antibody solution included 100 μl of 0.1 mM BS106.6 peptide, which was mixed with the antibody before adding to the blot. After a 60 min incubation, while rocking at room temperature, the blots were washed three times with 20 ml PBS containing 0.1% Tween 20. The blots were then incubated for 60 min while rocking with 5 ml of a 1:10000 dilution of alkaline phosphatase anti-rabbit conjugate (Tropix, Bedford Mass.). The blots were developed with 2 ml of NBT/BCIP substrate (Pierce One Step™, Pierce Chemical, Rockford, Ill.).

TABLE 4
Preparation of aqueous and fat portions of human milk samples

| | Sample | 10% | | 2 × Sample | Sodium |
|---|---|---|---|---|---|
| Sample | Volume | TCEP | PBS | Buffer | Carbonate |
| Milk Aqueous | 20 | 0 | 10 | 30 | 0 |
| Milk Aqueous | 20 | 5 | 0 | 30 | 8 |
| Milk Fat | 20 | 0 | 10 | 30 | 0 |
| Milk Fat | 20 | 5 | 0 | 30 | 8 |

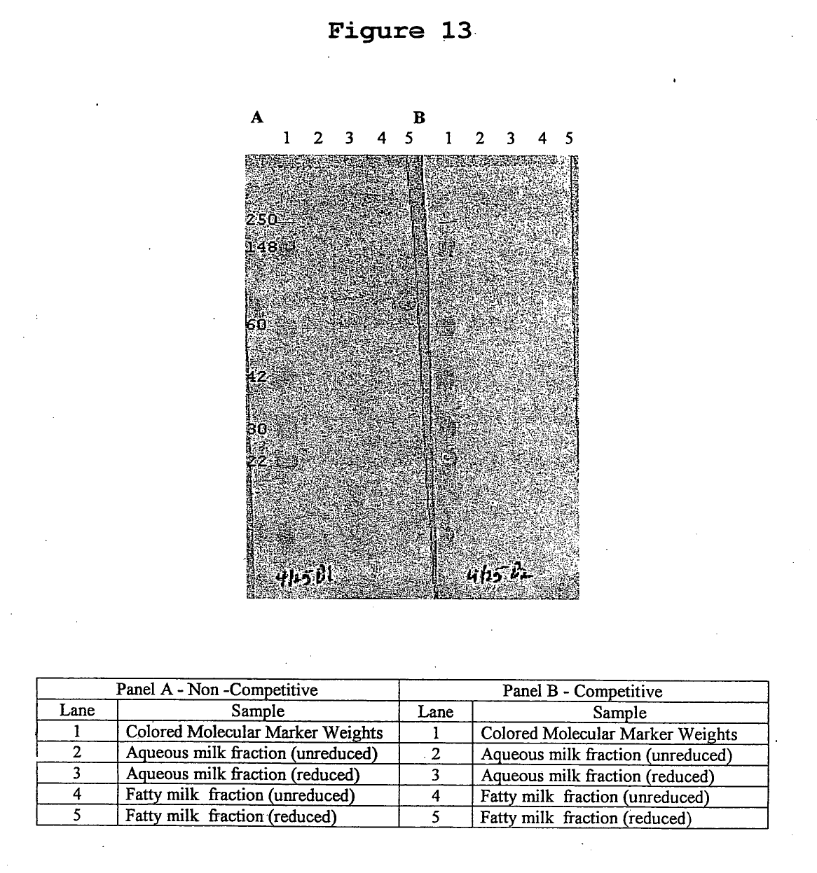

FIG. 13 shows that the affinity purified rabbit anti-BS106 antibody detects bands at several molecular weights, and that all of these bands disappear in the presence of BS106 peptide (SEQUENCE ID NO 26), suggesting that multiple proteins present in both aqueous and fatty fractions of milk contain amino acid sequence identical to or strongly related to that of BS106 peptide (SEQUENCE ID NO 20). In the absence of reducing agent the aqueous and fatty fractions show a high molecular weight band (>200 kD), and another band at about 120 kD (A, lanes 2 and 4). On reduction the band at 120 kD remains, however the high molecular weight band (>200 kD) disappears, and bands at 80 kD, 65 kD, and 40 kD appear (A, lanes 3 and 5). The sample containing the peptide for competition was run on blot B. The 80 kD and 65 kD bands appear there (B, lane 3) and are therefore considered not specific for BS106, unlike the 40 kD band.

The band at 40 kD is consistent with a single chain BS106 protein molecule containing O-linked glycosylation as predicted from its sequence (as described in Example 11b). The sole cysteine residue and its penultimate position in the sequence suggest that it forms disulfide links with other proteins in the course of its biological function. This is consistent with the disappearance of the high MW band (>200 kD) and the appearance of the 40 kD band after exposure to conditions which reduce the disulfide bonds.

The 120 kD band, however, is not affected by reduction. Apparently BS106 protein forms reduction resistant links with other proteins present in milk. One suggestion is that one or both of the lysines near the C-terminus may be coupled to other proteins by transglutamination.

C. Western Blotting of Biological Samples with BS106 Antibodies. Various biological samples were analyzed by Western blot under reducing and non-reducing conditions. Aqueous and fatty fractions of human milk were prepared as in Example 22D. To 6 μl of the milk aqueous fraction was added 24 μl of PBS and 30 μl of 2×SDS Sample Buffer (Novex, San Diego, Calif.) with and without 5% dithiothreitol. The milk fatty fraction (6% dispersion in PBS) and an extract of breast tumor tissue (from Example 16A) were similarly treated.

A sample of saliva was added to 18 μl of PBS and centrifuged for 10 min at 15,000×g. Twelve microliters (12 μl) of the supernate were added to 30 μl 2× Sample Buffer (Novex, San Diego, Calif.) with and without 5% dithiothreitol. Twelve microliters (12 μl) of a suspension of the sediment from the above saliva preparation in its original volume of PBS were similarly treated. To 3 μl of a sample of BS106 protein, affinity purified from human milk as in Example 22D, were added 27 μl PBS and 30 μl 2×SDS Sample Buffer (Novex, San Diego, Calif.) with and without 5% dithiothreitol.

All samples were heated 5 minutes at 100° C. and electrophoresed as described in Example 16A. After electrophoresis, the gels were blotted to nitrocellulose as directed by the manufacturer's instructions. The blots were blocked for 60 minutes with Iblock™ (0.2% in PBS containing 0.1% Tween-20). The blots were incubated for 60 minutes with 10 ml Iblock™ solution containing either 8.5 μl affinity purified rabbit anti-BS106 peptide (SEQUENCE ID NO 26) from Example 15B, or, 280 μl of the culture supernate containing H24C16 monoclonal antibody, both either with or without 50 μg BS106 peptide (SEQUENCE ID NO 26) added to compete for the antibody. After a 60 min incubation, while rocking at room temperature, the blots were washed three times with 20 ml of PBS containing 0.1% Tween 20. They were then incubated for 60 min while rocking with 5 ml of a 1:10000 dilution of alkaline phosphatase anti-rabbit or anti-mouse conjugate (Tropix, Bedford Mass.), and developed with 2 ml of NBT/BCIP substrate (Pierce One Step™, Pierce Chemical, Rockford, Ill.).

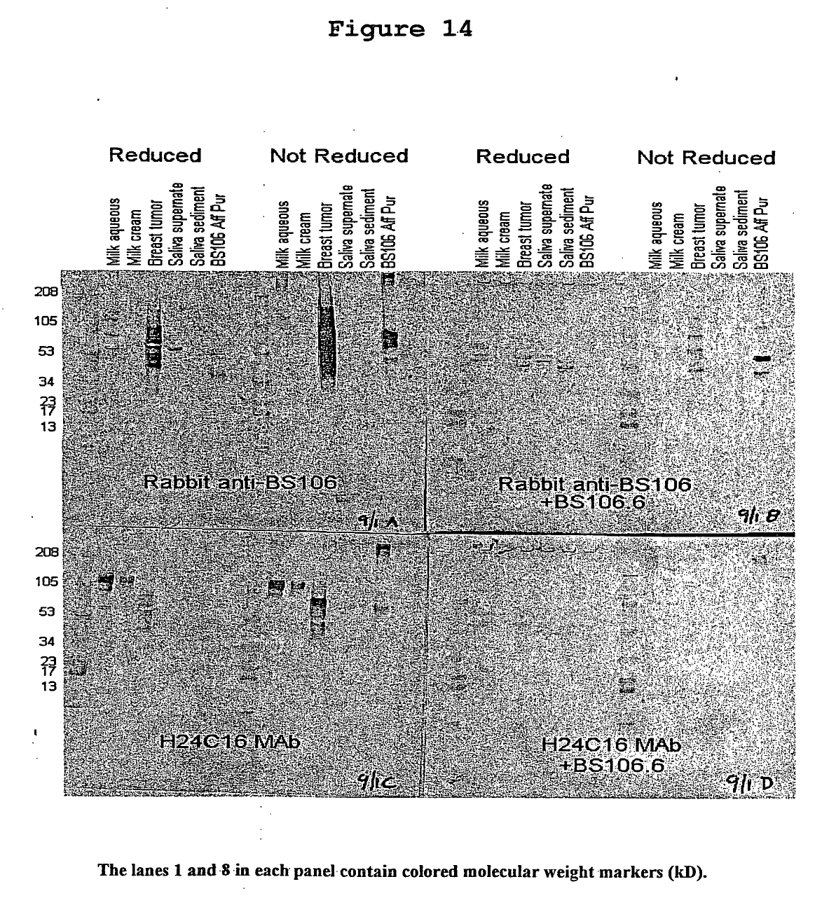

The results are shown in FIG. 14. According to the manufacturer, the colored MW markers move differently on Tricine gels than on the Tris-glycine gels used in Example 16A, resulting in different molecular weights attributed to them The >200 kD band is recognized by both the affinity purified rabbit antibody and the monoclonal antibody H24C16. The band is present (under non-reducing conditions) in the aqueous milk fraction, breast tumor, saliva supernate, and the BS106 protein affinity purified from milk.

The 120 kD band is also recognized by both the affinity purified rabbit antibody and the monoclonal antibody H24C16. The band is observed in the milk samples including the aqueous milk fraction, the fatty fraction, and the BS106 protein affinity purified from milk. However, it does not appear to be present in the saliva samples and is difficult to conclude its presence in the breast tumor sample due to the intense staining. The breast tumor specimen and the BS106 protein affinity purified from milk further contain some material ranging in molecular weight from 45-70 kD that is recognized by both antibodies.

Upon reduction, the high molecular weight band (>200 kD) and the bands of molecular weight (45-70 kD) disappear and a band at 40 kD is produced. This is best observed with the BS106 protein affinity purified from milk, as the sample is cleaner and more concentrated. The 120 kD band is unaffected by the reduction.

These results are consistent with the presence of a disulfide-linked complex involving BS106 with a molecular weight greater than 200 kD. BS106 also appears to be involved in a non-reduceable complex with a molecular weight of approximately 120 kD.

### Example 17

### EIA Microtiter Plate Assay

The immunoreactivity of monoclonal antibodies or antiserum obtained from rabbits or mice as described in Example 14 was determined by means of a microtiter plate EIA, as follows. Microtiter plates were coated with either synthetic peptides or semi-purified expressed protein material (purified as described in Example 11b, Part E). Briefly, synthetic peptides, SEQUNENCE ID NO 21-33, prepared as described in Example 10, were dissolved in carbonate buffer (50 mM, pH 9.6) to a final concentration of 2 μg/ml. Semi-purified expressed protein was diluted in PBS 1:20. Next, 100 μl of the peptide or protein solution were placed in each well of an Immulon 2® microtiter plate (Dynex Technologies, Chantilly, Va.). The plate was incubated overnight at room temperature and then washed four times with deionized water. The wells were blocked by adding 125 μl of a suitable protein blocking agent, such as Superblock® (Pierce Chemical Company, Rockford, Ill.), to each well and then immediately discarding the solution. This blocking procedure was performed three times. Monoclonal antibodies or antisera obtained from immunized rabbits or mice, prepared as previously described, were diluted in a protein blocking agent (e.g., a 3% Superblock® solution) in PBS containing 0.05% Tween-20® (monolaurate polyoxyethylene ether) (Sigma Chemical Company, St. Louis, Mo.) and 0.05% sodium azide at dilutions of 1:100, 1:500, 1:2500, 1:12,500, and 1:62,500 (polyclonal antisera) or 1:10, 1:100, 1:1000, and 1:10,000 (hybridoma supernatants) and placed in each well of the coated microtiter plate. The wells then were incubated for three hours at room temperature. Each well was washed four times with deionized water. One hundred microliters (100 μl) of alkaline phosphatase-conjugated goat anti-rabbit IgG or goat anti-mouse IgG antiserum (Southern Biotech, Birmingham, Ala.) diluted 1:2000 in 3% Superblock® solution in phosphate buffered saline containing 0.05% Tween 20® and 0.05% sodium azide, were added to each well. The wells were incubated for two hours at room temperature. Next, each well was washed four times with deionized water. One hundred microliters (100 μl) of paranitrophenyl phosphate substrate (Kirkegaard and Perry Laboratories, Gaithersburg, Md.) then were added to each well. The wells were incubated for thirty minutes at room temperature. The absorbance at 405 nm was read in each well. Positive reactions were identified by an increase in absorbance at 405 nm in the test well above that absorbance given by a non-immune serum (negative control). A positive reaction was indicative of the presence of detectable anti-BS106 antibodies. Titers of the anti-peptide antisera or monoclonal antibodies were calculated from the previously described dilutions of antisera and defined as the calculated dilution, where A_{405 nm}=0.5 OD. Table 5 shows titers of the monoclonal antibody culture supernatants which were generated using synthetic peptide (peptide used as immunogen) or the semi-purified expressed protein material (BS106 M/H).

TABLE 5
Binding Properties of Monoclonal Antibodies

| | | | | Peptide | | |
|---|---|---|---|---|---|---|
| | | | Peptide Immunogen | Immunogen | BS106M/H | BS106M/H |
| Study | Experiment | Hybridoma | SEQUENCE ID NO | Titer | Titer | K_{d}(app) |
| 392 | 3 | H6C52 | 26 | 800 | 660 | 118 |
| 392 | 3 | H9C29 | 26 | 250 | 250 | 231 |
| 392 | 3 | H39C51 | 26 | 380 | 350 | 241 |
| 392 | 3 | H80C32 | 26 | 900 | 900 | 70 |
| 392 | 7 | H84C55 | 26 | 600 | 650 | 170 |
| 392 | 7 | H121C68 | 26 | 100 | 270 | 143 |
| 392 | 7 | H144C20 | 26 | 600 | 700 | 164 |
| 392 | 7 | H24C16 | 26 | 300 | 125 | 89 |
| 392 | 14 | H17C77 | 23 | <10 | <10 | ND |
| 392 | 14 | H59C34 | 23 | 300 | 100 | 358 |
| 392 | 14 | H121C19 | 23 | 90 | <10 | ND |
| 392 | 14 | H131C50 | 23 | 800 | <10 | ND |
| 392 | 14 | H147C64 | 23 | <10 | <10 | ND |
| 392 | 14 | H184C51 | 23 | 325 | 75 | 132 |
| 392 | 14 | H195C62 | 23 | 700 | 100 | 448 |
| 392 | 14 | H212C68 | 23 | 825 | 100 | 193 |
| 392 | 14 | H218C31 | 23 | 725 | 500 | 509 |
| 392 | 15 | H119C17 | 26 | 760 | 70 | 201 |
| 392 | 15 | H150C31 | 26 | 50 | 60 | 478 |
| 392 | 15 | H182C63 | 26 | 800 | <10 | ND |
| 392 | 15 | H188C82 | 26 | 700 | 100 | 271 |
| 392 | 15 | H203C46 | 26 | 540 | 100 | 236 |
| 392 | 15 | H205C33 | 26 | 1500 | <10 | 28 |
| 400 | 3 | H24C29 | 29 | 900 | ND | ND |
| 400 | 3 | H51C42 | 29 | 3600 | ND | ND |
| 400 | 3 | H62C52 | 29 | 40 | ND | ND |
| 400 | 3 | H102C47 | 29 | ND | ND | ND |
| 400 | 3 | H30C35 | 29 | ND | ND | ND |
| 400 | 3 | H26C39 | 29 | ND | ND | ND |
| 400 | 4 | H16C70 | 29 | 680 | ND | ND |
| 400 | 4 | H45C35 | 29 | 2800 | ND | ND |
| 400 | 4 | H62C59 | 29 | 350 | ND | ND |
| 400 | 4 | H76C43 | 29 | ND | ND | ND |
| 400 | 4 | H4C33 | 29 | ND | ND | ND |
| 400 | 4 | H57C43 | 29 | ND | ND | ND |
| 400 | 4 | H44C56 | 29 | ND | ND | ND |
| 400 | 4 | H94C45 | 29 | ND | ND | ND |
| 400 | 4 | H34C78 | 29 | ND | ND | ND |
| 400 | 4 | H2C15 | 29 | ND | ND | ND |

In addition to titers, apparent affinities [K_{d}(app)] were also determined for some of the antibodies. In this case, pooled and dialysed semi-purified expressed protein material (purified as described in Example 11b, Part E) were prepared at dilutions of 1:3, 1:9, 1:27, 1:81, 1:243, 1:729, 1:2187, 1:6561, and 1:19683 in PBS and 100 μl were placed in each well of an Immulon 2® High Binding microtiter plate (Dynex Technologies, Chantilly, Va.). The plate was incubated for 14-18 hours at room temperature and then washed four times with deionized water. The wells were blocked by adding 125 μl of Superblock® (Pierce Chemical Company, Rockford, Ill.) to each well and then immediately discarding the solution. The blocking procedure was performed three times. Monoclonal antibodies obtained from hybridoma culture supernatant or antisera obtained from immunized rabbits or mice, as described hereinabove in Example 14, were diluted at an appropriate dilution in a protein blocking agent (i.e., 3% Superblock® solution) in PBS containing 0.05% Tween-20 (monolaurate polyoxyethylene ether) (Sigma Chemical Company, St. Louis, Mo.) and 0.05% sodium azide and placed in each well of the coated microtiter plate. The wells were then incubated for one hour at room temperature. Each well was washed four times with deionized water. One hundred microliters (100 μl) of alkaline phosphatase-conjugated goat anti-mouse IgG or goat anti-rabbit IgG (Southern Biotech, Birmingham, Ala.), diluted 1:2000 in 3% Superblock® solution were added to each well. The wells were incubated for one hour at room temperature. Next, each well was washed four times with deionized water. One hundred microliters (100 μl) of para-nitrophenyl phosphate substrate (Kirkegaard and Perry Laboratories, Gaithersburg, Md.) then were added to each well. The wells were incubated for thirty minutes at room temperature. The absorbance at 405 nm was read in each well. EIA microtiter plate assay results were used to derive the apparent dissociation constants [K_{d(app)}] based on an analog of the Michaelis-Menten equation (V. Van Heyningen, Methods in Enzymology, Vol. 121, p. 472 (1986) and further described in X. Qiu, et al, Journal of Immunology, Vol. 156, p. 3350 (1996)):

$$[ Ag - Ab ] = {[ Ag - Ab ]}_{max} \times \frac{[ Ab ]}{[ Ab ] + {K}_{d}}$$

where [Ag-Ab] was the antigen-antibody complex concentration, [Ag-Ab]ₘₐₓ was the maximum complex concentration, [Ab] was the antibody concentration, and K_{d} was the dissociation constant. During the curve fitting, the [Ag-Ab] was replaced with the background subtracted value of the OD_{405 nm} at the given concentration of Ab. Both K_{d}, which corresponds to K_{d}(app), and [OD_{405 nm}]ₘₐₓ, which corresponds to the [Ag-Ab]ₘₐₓ, were treated as fitted parameters. The software program Origin was used for the curve fitting. Apparent affinities [K_{d}(app)] were determined for the monoclonal antibodies (Table 6) and the rabbit polyclonal antibodies (Table 5) using semi-purified expressed protein material (BS106 M/H).

TABLE 6
Binding Properties of Rabbit Polyclonal Antibodies

| | | Peptide Conjugated | BS106M/H |
|---|---|---|---|
| Rabbit # | Peptide Immunogen | to a Carrier Protein? | K_{d}(app) |
| 11537 | SEQUENCE ID NO 23 | no | 287 |
| 11538 | SEQUENCE ID NO 23 | no | 197 |
| 11539 | SEQUENCE ID NO 23 | no | 270 |
| 11540 | SEQUENCE ID NO 23 | no | 166 |
| 11492 | SEQUENCE ID NO 23 | yes | 370 |
| 11494 | SEQUENCE ID NO 26 | yes | 576 |

### Example 18

### Coating of Solid Phase Particles

A. Coating of Microparticles with Antibodies Which Specifically Bind to BS106 Antigen. One hundred microliters (100 μl) of carboxymethyl latex microspheres (interfacial Dynamics 1.0 micron diameter, 4.2% solids) were suspended in 1.0 ml of 5 mM morpholinoethanesulfonic acid (MES) buffer (pH 5.6) with 0.1% Triton X 100. The mixture was vortexed and then centrifuged for 1 minute at approximately 500×g to sediment the microspheres. The supernatant was discarded and the pellet containing the microspheres was resuspended in 1.0 ml of the MES buffer. Forty microliters (40 μl) of antibody (affinity purified rabbit anti-BS106 antibody at 3 mg/ml in PBS) and 20 μl of 1.0 mM 1-ethyl-3-(3-dimethylaminopropyl)carbodiimide) (EDAC) were added to the resuspended microspheres. The mixture was vortexed and rotated for 5 h at room temperature. To the mixture was added 400 μl of BSA buffer (0.2% bovine serum albumin (Sigma, St. Louis, Mo.) and 0.08% Tween-20® (Sigma, St. Louis, Mo.) in MEIA buffer (Abbott Laboratories, Abbott Park, Ill.). The mixture was vortexed, centrifuged as above, and the supernatant was discarded. The pellet was washed twice with 1.0 ml of the BSA buffer prior to resuspension in 400 μl of the same buffer.

B. Coating of ¼ Inch Beads. Antibodies which specifically bind to BS106-antigen also may be coated on the surface of 1/4 inch polystyrene beads by routine methods known in the art (Snitman et al, U.S. Pat. No. 5,273,882, incorporated herein by reference) and used in competitive binding or EIA sandwich assays.

Polystyrene beads first are cleaned by ultrasonicating them for about 15 seconds in 10 mM carbonate buffer at pH 8.0. The beads then are washed in deionized water until all fines are removed. Beads then are immersed in an antibody solution in 10 mM carbonate buffer, pH 8 to 9.5. The antibody solution can be as dilute as 1 μg/ml in the case of high affinity monoclonal antibodies or as concentrated as about 500 μg/ml for polyclonal antibodies which have not been affinity purified. Beads are coated for at least 12 hours at room temperature, and then they are washed with deionized water. Beads may be air dried or stored wet (in PBS, pH 7.4). They also may be overcoated with protein stabilizers (such as sucrose) or protein blocking agents used as non-specific binding blockers (such as irrelevant proteins, Carnation skim milk, Superblock®, or the like).

### Example 19

### Microparticle Enzyme Immunoassay (MEIA)

BS106 antigens are detected in patient test samples by performing a standard antigen competition EIA or antibody sandwich EIA and utilizing a solid phase such as microparticles (MEIA). The assay can be performed on an automated analyzer such as the IMx® Analyzer (Abbott Laboratories, Abbott Park, Ill.).

A. Antibody Sandwich EIA. Briefly, samples suspected of containing BS106 antigen are incubated in the presence of anti-BS106 antibody-coated microparticles (prepared as described in Example 18) in order to form antigen/antibody complexes. The microparticles then are washed and an indicator reagent comprising an antibody conjugated to a signal generating compound (i.e., enzymes such as alkaline phosphatase or horseradish peroxide) is added to the antigen/antibody complexes or the microparticles and incubated. The microparticles are washed and the bound antibody/antigen/antibody complexes are detected by adding a substrate (e.g., 4-methyl umbelliferyl phosphate (MUP), or OPD/peroxide, respectively), that reacts with the signal generating compound to generate a measurable signal. An elevated signal in the test sample, compared to the signal generated by a negative control, detects the presence of BS106 antigen. The presence of BS106 antigen in the test sample is indicative of a diagnosis of a breast disease or condition, such as breast cancer.

B. Competitive Binding Assay. The competitive binding assay uses a peptide or protein that generates a measurable signal when the labeled peptide is contacted with an anti-peptide antibody coated microparticle. This assay can be performed on the IMx® Analyzer (Abbott Laboratories, Abbott Park, Ill.). The labeled peptide is added to the BS106 antibody-coated microparticles (prepared as described in Example 18) in the presence of a test sample suspected of containing BS106 antigen, and incubated for a time and under conditions sufficient to form labeled BS106 peptide (or labeled protein)/bound antibody complexes and/or patient BS106 antigen/bound antibody complexes. The BS106 antigen in the test sample competes with the labeled BS106 peptide (or BS106 protein) for binding sites on the microparticle. BS106 antigen in the test sample results in a lowered binding of labeled peptide and antibody coated microparticles in the assay since antigen in the test sample and the BS106 peptide or BS106 protein compete for antibody binding sites. A lowered signal (compared to a control) indicates the presence of BS106 antigen in the test sample. The presence of BS106 antigen suggests the diagnosis of a breast disease or condition, such as breast cancer.

The BS106 polynucleotides and the proteins encoded thereby which are provided and discussed hereinabove are useful as markers of breast tissue disease, especially breast cancer. Tests based upon the appearance of this marker in a test sample such as blood, plasma or serum can provide low cost, non-invasive, diagnostic information to aid the physician to make a diagnosis of cancer, to help select a therapy protocol, or to monitor the success of a chosen therapy. This marker may appear in readily accessible body fluids such as blood, urine or stool as antigens derived from the diseased tissue which are detectable by immunological methods. This marker may be elevated in a disease state, altered in a disease state, or be a normal protein of the breast, which appears in an inappropriate body compartment.

### Example 20

### Immunohistochemical Detection of BS106 Protein

Antiserum against a BS106 synthetic peptide derived from the consensus peptide sequence (SEQUENCE ID NO 20) described in Example 14, above, is used to immunohistochemically stain a variety of normal and diseased tissues using standard proceedures. Briefly, frozen blocks of tissue are cut into 6 micron sections, and placed on microscope slides. After fixation in cold acetone, the sections are dried at room temperature, then washed with phosphate buffered saline and blocked. The slides are incubated with the antiserum against a synthetic peptide derived from the consensus BS106 peptide sequence (SEQUENCE ID NO 20) at a dilution of 1:500, washed, incubated with biotinylated goat anti-rabbit antibody, washed again, and incubated with avidin labeled with horseradish peroxidase. After a final wash, the slides are incubated with 3-amino-9-ethylcarbazole substrate which gives a red stain. The slides are counterstained with hematoxylin, mounted, and examined under a microscope by a pathologist.

### Example 21

### Fluorescence Polarization Immunoassay of BS106

A. Conjugation of BS106 Peptide to Fluorescein Derivatives. The peptide, SEQUENCE ID NO 26 was coupled to fluorescein-5-maleimide using the following procedure. One and one tenth milligram (1.1 mg) of Tris-carboxyethylphosphine hydrochloride and 0.3 μmole of fluorescein-5-maleimide (Molecular Probes, Eugene, Oreg.) were dissolved in 17 μl of dimethylformamide. This mixture was added to 0.75 μmoles of SEQUENCE ID NO 26 peptide in 50 μl of 600 mM triethanolamine buffer (pH 7.6). The reaction was allowed to proceed for 30 minutes at room temperature after which the mixture was passed over a Biogel P2 column (8 ml) to separate the higher molecular weight conjugate from the unreacted fluorescein. The column was equilibrated with a 10-fold dilution of PBS in water. The first colored band was collected, giving 500 μl of yellow solution.

The peptide, SEQUENCE ID NO 26 was coupled to 5-carboxyfluorescein using the following procedure. Three tenths micromole (0.3 μmole) 5-carboxyfluorescein NHS ester (Molecular Probes, Eugene, Oreg.) was dissolved in 21 μl of dimethylformamide. This mixture was added to 0.75 μmole of SEQUENCE ID NO 26 peptide in 50 μl of 600 mM triethanolamine buffer (pH 7.6). The reaction was allowed to proceed for 30 minutes at room temperature after which the mixture was passed over a Biogel P2 column (8 ml) to separate the higher molecular weight conjugate from the unreacted fluorescein. The column was equilibrated with a 10-fold dilution of PBS in water. The first colored band was collected, giving 500 μl of yellow solution.

B. Purification of BS106 Fluorescein Conjugates. The isomeric conjugates in the above products were separated by isoelectric focusing using prepared gels and buffers from Novex (Novex, San Diego, Calif.). To 150 μl IEF sample buffer containing 1.5 mg dithiothreitol was added 150 μl of the product of the above conjugation reaction. The mixtures were placed in the sample compartments of the IEF gels from which the lane dividers had been removed to give a single well for each gel. The gels were electrophoresed for 60 minutes at 100 V and then for 60 minutes at 200 V, during which each of the samples were visibly resolved into several narrow bands. The gel cassettes were opened, and the major bands were cut out with a razor blade and transferred to culture tubes containing 1.0 ml of PBS. After 3 hours of incubation at room temperature, most of the color appeared to be in solution. The fluorescein concentration of each sample was measured by absorbance at 492 nm, assuming an extinction coefficient of 73000 M⁻¹cm⁻¹.

The fluorescence intensity and polarization of the conjugates were measured using the fluorescence polarization format of the IMx® Analyzer (Abbott Laboratories, Abbott Park, Ill.). Each conjugate was diluted to 10 nM and the measurements were taken after which 1.0 μl of the 3.0 mg/ml affinity purified rabbit anti-BS106 was added. The mixture was vortexed and the measurement was repeated. Results are presented in Table 7.

Table 7 lists the approximate isoelectric point (pI), absorbance measurement (A₄₉₂), fluorescence intensity (Intensity), and positive (Pol+) and negative (Pol−) polarization values of each conjugate. Conjugates N1-N5 were derived from 5-carboxyfluorescein while M1-M3 were derived from fluorescein-5-maleimide. Isoelectric pH standards were not used, therefore the pIs listed were estimated from comparison with a published chart (Novex, San Diego, Calif.) and are only approximate. A₄₉₂ refers to the absorbance of the undiluted solution, and is a measure of the yield of the procedure. Intensity is in arbitrary fluorescence units. Pol− is the fluorescence polarization in mP units in the absence of anti BS106 antibody, and Pol+ is the polarization after addition of the antibody.

It is first notable that conjugates N5 and M3, those with the lowest pI, show no change in fluorescence polarization on addition of the antibody. These compounds are attributed to be the unconjugated fluorescein derivatives. Conjugates M1 and M2, both of which show significant elevation of the fluorescence polarization in the presence of antibody, are consistent with derivatization at either of the two cysteines in the peptide. The situation for N1-N4 is less clear. Since the SEQUENCE ID NO 26 peptide contains 3 amines available for reaction with the fluorescein derivative (two lysines plus the N-terminal), only three major conjugates should be present. However, all 4 show significant binding to the antibody.

TABLE 7
Fluorescence Intensity and Polarization Results

| Conjugate | pI | A₄₉₂ | Intensity | Pol− | Pol+ |
|---|---|---|---|---|---|
| N1 | 6.5 | 0.2406 | 4357 | 48.59 | 95.58 |
| N2 | 6.0 | 0.2464 | 4370 | 64.55 | 182.47 |
| N3 | 5.5 | 0.2519 | 4171 | 47.92 | 114.54 |
| N4 | 5.3 | 0.2589 | 3391 | 53.92 | 149.82 |
| N5 | 3.5 | 0.2693 | 4507 | 31.07 | 31.61 |
| M1 | 6.5 | 0.1884 | 5329 | 57.83 | 142.07 |
| M2 | 5.7 | 0.1323 | 4886 | 53.77 | 136.91 |
| M3 | 3.5 | 0.2169 | 8044 | 24.80 | 24.15 |

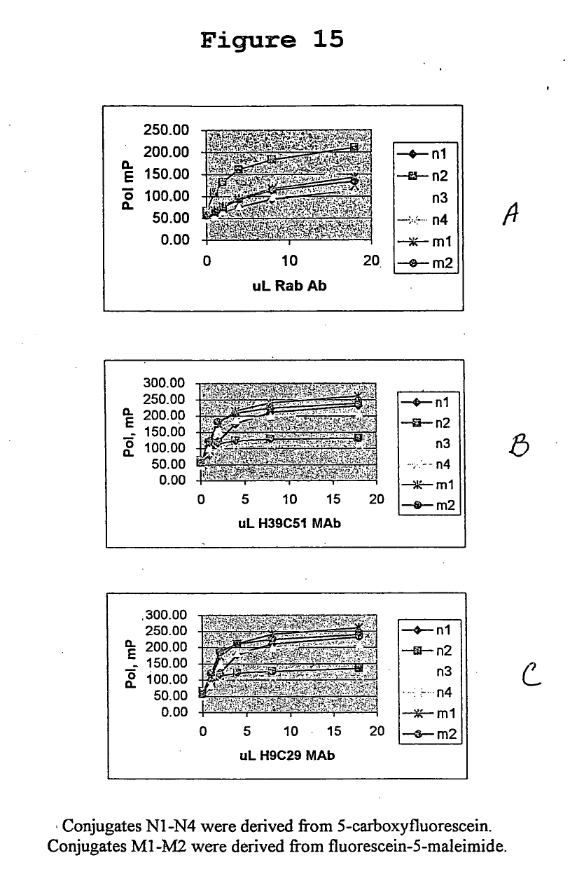

These conjugates were useful in testing antibodies directed against the C-terminal end of BS106, and in competitive immunoassays measuring BS106 concentrations in biological fluids. In FIG. 15, 1.0 ml samples containing 2.0 nM of the conjugates were titrated with the affinity purified rabbit anti-BS 106 and the mouse monoclonal antibodies H39C51 and H9C29. The polarizations increased rapidly with addition of antibody, leveling off as the solutions become saturated. The maximum polarization is characteristic of both the conjugate and the antibody, depending on the environment of the fluorescein moiety in the complex. Interestingly, conjugate N2, which showed the highest limiting polarization with the rabbit antibody, showed one of the lowest with both the monoclonal antibodies. Although they were derived from separate fusions, the two monoclonal antibodies showed nearly identical titration plots with all the conjugates, suggesting strong similarity of the binding sites.

C. Fluorescence Polarization Immunoassay of BS106 Using Fluorescein Conjugate. A competitive assay was carried out using the SEQUENCE ID NO 26 peptide-fluorescein conjugate (M1) as competitor. The antibody used was monoclonal antibody H24C16. The assay was set up such that the antibody [0.4 μg/ml in 400 μl of FPIA diluent (Abbott Laboratories, Abbott Park, Ill.)] and the sample or standard were mixed and incubated at room temperature for 20 minutes. The competitor (conjugate M1) was then added and the polarization measured. The standard curve was obtained using known amounts of SEQUENCE ID NO 26 peptide. The indicated test samples were products of affinity purification of human milk (Example 22D). The results are presented in Table 8.

TABLE 8
Competitive Assay Results

| | | | pmoles from | μM sample |
|---|---|---|---|---|
| Sample | μl | Pol, mP | curve | from curve |
| Standard | 0.0 | 159.24 | 0.00 | |
| 1.0 μM | 0.5 | 140.99 | 0.53 | 1.060 |
| SEQUENCE ID NO 26 | 1.0 | 129.93 | 0.92 | 0.920 |
| | 2.0 | 110.54 | 2.11 | 1.055 |
| | 5.0 | 94.72 | 4.88 | 0.976 |
| | 10.0 | 86.91 | 10.08 | 1.008 |
| 11B | 0.1 | 133.77 | 0.77 | 7.740 |
| | 1.0 | 92.83 | 5.60 | 5.598 |
| | 10.0 | 79.75 | | |
| 24A | 0.1 | 131.44 | 0.86 | 8.630 |
| | 1.0 | 93.44 | 5.35 | 5.345 |
| 24B | 0.1 | 158.26 | 0.04 | 0.390 |
| | 1.0 | 143.75 | 0.45 | 0.446 |
| | 10.0 | 96.45 | 4.35 | 0.435 |
| 26A | 10.0 | 145.95 | 0.38 | 0.038 |
| 26B | 10.0 | 152.32 | 0.21 | 0.021 |

The polarization results were evaluated using a four parameter log-logit curve fitting routine to give the pmoles of analyte in the cuvette, which was divided by the volume of sample added to give its concentration in 1M. The results are calculated with the assumption that the assay is insensitive, to differences between BS106 protein isolated from milk and the SEQUENCE ID NO 26 peptide-fluorescein conjugate, which competes with it for binding sites on the antibody. Isolation and measurement of the concentration of BS106 protein by an unambiguous means would provide standards, which could be used in this immunoassay format to give reliably accurate concentrations of unknown samples.

### Example 22

### Affinity Purification of BS106

A. Preparation of Amino-Link™ Affinity Column for Purification of BS106. Affinity purified rabbit anti-BS106 antibody was coupled to Amino-Link™ gel (Pierce Chemical, Rockford, Ill.) as directed by the manufacturer's instructions. Briefly, 6.5 mg of affinity purified rabbit anti-BS106 antibody (in 4.5 ml of PBS) were passed through a desalting column equilibrated with citrate/carbonate buffer (pH 10). The eluent was placed on a column containing 2.0 ml AminoLink gel equilibrated with citrate/carbonate buffer. The column containing the antibody and gel was rotated for 4 hours at room temperature, drained, and washed with 5 ml of PBS. After draining the column, 2.0 ml of PBS and 40 μl of 5 M sodium cyanoborohydride were added. The column was rotated for 4 hours at room temperature, drained, and washed with 5 ml of Pierce wash buffer. The column was ready for use.

B. Preparation of Sulfo-Link™ Affinity Column for Purification of BS106. Affinity purified rabbit anti-BS106 antibody was coupled to Sulfo-Link™ gel (Pierce Chemical, Rockford, Ill.) as directed by the manufacturer's instructions. Briefly, 8.6 mg of affinity purified rabbit anti-BS106 antibody (in 2 ml of 100 mM sodium phosphate, 5 mM EDTA, pH 6.0) were reduced with 14 mg of mercaptoethylamine hydrochloride for 90 minutes at 37° C. The reaction mixture was desalted on a 10 ml Kwik™ column and placed into vacuum degassed coupling buffer. The reduced protein was transferred to a 2 ml Sulfo-Link™ column previously washed with degassed coupling buffer. The gel-protein mixture was mixed by inversion for 15 minutes at room temperature, and allowed to settle for 30 minutes. The buffer containing uncoupled protein was collected and the column was washed with 6 ml of coupling buffer. The remaining binding sites on the column were blocked by agitating the gel for 15 minutes with 2.0 ml of coupling buffer containing 15.8 mg cysteine hydrochloride. The gel was allowed to settle for 30 minutes, and was then drained, and washed with 10 ml of PBS. The column was ready for use.

C. Affinity Purification of BS106 from Transient Transfection. Fifteen milliliters (15 ml) of culture supernate from the transient transfection of BS106 (as described in Example 11c) were passed through the rabbit anti-BS106 Amino-Link™ column. The column was washed with 5 ml of PBS and the protein was eluted with 4 ml of 100 mM glycine, pH 2.1. The eluting protein was collected in tubes containing 300 μl of 1.0 M Tris base for neutralization.

The protein was further purified using a lectin which binds O-linked sugars. The eluate was passed through a column containing 5.0 ml of jacalin agarose equilibrated in PBS (Sigma, St. Louis, Mo.) to bind the glycosylated protein. The column was washed with 20 ml of PBS, and the product was eluted with 10% melibiose (Sigma, St. Louis, Mo.) in PBS. The product, which was concentrated in fractions at 4.0-7.0 ml, was passed through a 10 ml desalting column equilibrated with PBS. The desalted product was concentrated to 0.5 ml using a Centricon 10 centrifugal concentrator (Amicon, Beverly Mass.) resulting in an absorbance reading at 280 nm of 0.91.

D. Affinity Purification of BS106 from Human Milk. One hundred milliliters (100 ml) of frozen human milk was thawed, transferred to two 50 ml conical tubes, and centrifuged for 60 min at 2000×g. The tubes were punctured near the bottom to drain the turbid aqueous portion from the fatty layer at the top and the small amount of solid at the bottom. Fifty milliliters of the aqueous portion was passed through the rabbit anti-BS106 Amino-Link™ column. The column was washed with PBS until the absorbance at 280 nm was below 0.005. The protein was eluted with 100 mM glycine, pH 2.15. The eluent was collected in 400 μl fractions, which were neutralized with 50 μl of 1.0 M Tris base. The fractions with an absorbance reading at 280 nm of at least 0.03 were combined and passed through a column containing 0.5 ml jacalin agarose (Sigma, St. Louis, Mo.) equilibrated with PBS. This column was washed with 5 ml of PBS and the product was eluted with 10% melibiose in PBS. The fraction eluting at 400 μl to 1400 μl was passed through a desalting column with PBS, and then concentrated with a Microcon 10 centrifugal concentrator (Amicon, Beverly Mass.) to 250 μl. The absorbance at 280 nm of the solution was 0.080.

1. A purified multimeric polypeptide antigen (MPA) comprising at least one BU106 polypeptide said polypeptide having at least 50% identity to the polypeptides in SEQ ID NOS: 20-33, and at least other polypeptide together forming a molecular weight of at least 200 Kd.

2. The MPA of claim 1, wherein said polypeptides are complexed via a disulfide bond.

3. A purified multimeric polypeptide antigen (MPA) comprising at least one BS106 polypeptide said polypeptide having at lest 50% identity to the polypeptides in SEQ ID NOS: 20-33 complexed with another peptide to form a non-reductable complex having a molecular weight of approximately 120 Kd.

4. A method of detecting the presence of a multimeric polypeptide antigen (MPA) in a test sample suspected of containing said MPA, wherein said MPA comprises at least one BU106 polypeptide and, at least, one other polypeptide, wherein said MPA has a molecular weight greater than 200 Kd, said method comprising the steps of:
(a) contacting said test sample with at least one antibody specific for at least one epitope of said MPA for a time and under conditions sufficient to allow the formation of antigen/antibody complexes; and
(b) detecting said complexes, wherein presence of said complexes wherein said detection indicates the presence of said MPA in said test sample.

5. A method of detecting the presence of a multimeric polypeptide antigen (MPA) in a test sample suspected of containing said MPA, wherein said MPA comprises at least one BU106 polypeptide and, at least, one other polypeptide, wherein said MPA has a molecular weight greater than approximately 120 Kd, said method comprising the steps of:
(a) contacting said test sample with at least one antibody specific for at least one epitope of said MPA for a time and under conditions sufficient to allow the formation of antigen/antibody complexes; and
(b) detecting said complexes, wherein detection of said complexes indicates the presence of said MPA in said test sample.

6. A purified polypeptide antigen comprising BUS 106 polypeptide, said polypeptide having at least 50% identity to those polypeptides selected from the group consisting of SEQ ID NOS: 20-33 with at least two carbohydrate covalently linked at multiple sites, said sites selected from the group consisting of serine residue sites and threonine residue sites.

7. A purified polypeptide antigen comprising BS106 polypeptide, said polypeptide having at least 50% identity to those polypeptides selected from the group consisting of SEQ ID NOS: 20-33 and at least one carbohydrate.

8. A purified multimeric polypeptide antigen (MPA) comprising at least one BU106 polypeptide and at least one other polypeptide together forming a complex, said complex having molecular weight of approximately 50 to 80 kD.

9. An antibody which will specifically bind to the MPA of claim 1.

10. An antibody which will specifically bind to the MPA of claim 3.

11. An antibody which will specifically bind to the antigen of claim 6.

12. An antibody which will specifically bind to the antigen of claim 7.

13. An antibody which will specifically bind to the antigen of claim 8.

14. The cell line designated 106C1.

15. The method of claim 4, wherein said detection of said complex is indicative of breast disease.

16. The method of claim 5, wherein said detection of said complex is indicative of breast disease.

17. A diagnostic kit comprising a panel of breast markers comprising: at least one BS106 marker and at least one other breast-specific marker.

18. An antibody which specifically binds to a BU106 polypeptide wherein said BU106 polypeptide has at least 50% identity to SEQUENCE ID NOS. 20-33.

19. A method of detecting BU106 comprising:
(a) providing a patient sample;
(b) analyzing said sample for a polypeptide having a molecular weight selected from the group consisting of approximately 200 Kd and 120 Kd;
(c) detecting the presence of said polypeptide have said molecular weight.

20. A method for detecting cancer cells, if present, in a tissue sample from a human patient, the method comprising contacting a tissue sample from a patent with a recognition agent for BU106, and detecting binding of the recognition agent to BU106 protein in the prepared sample as an indication of the presence of cancer cells in the tissue sample.

21. A method according to claim 20 which includes the steps of obtaining from a patient a tissue sample to be tested for the presence of cancer cells; and producing a prepared sample in a sample preparation process; prior to contacting the prepared sample with a recognition agent that reacts with BU106 protein.

22. The method of claim 21 wherein binding of said recognition agent to BU106 protein in sample is detected by immunohistochemical analysis.

23. The method of claim 22, wherein the sample preparation process comprises contacting the tissue with fixative and producing a thin seciton suitable for immunohistochemical analysis.

24. The method of claim 20 wherein said BU106 is in complexed form.
